SEQUENCE LISTING (44 sequences)

SEQ ID NO: 1 - protein, 133 aa, Homo sapiens [sequence omitted]

SEQ ID NO: 2 - protein, 132 aa, synthetic construct [sequence omitted]

SEQ ID NO: 3 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 134 aa, synthetic construct
  VARIANT 6: note = nonnatural amino acid
  VARIANT 32: note = nonnatural amino acid
  VARIANT 36: note = nonnatural amino acid
  VARIANT 75: note = nonnatural amino acid
MAPTSXSTKKTQLQLEHLLLDLQMILNGINNXKNPXLTRMLTFKFYMPKKATELKHLQCLEEELKPLEEVLNLAXSKNFHLRPRDLISNINVIVLELKGSETTFMCEYADETATIVEFLNRWITFSQSIISTLT

SEQ ID NO: 5 - protein, 133 aa, synthetic construct
  VARIANT 5: note = nonnatural amino acid
  VARIANT 31: note = nonnatural amino acid
  VARIANT 35: note = nonnatural amino acid
  VARIANT 74: note = nonnatural amino acid
MPTSXSTKKTQLQLEHLLLDLQMILNGINNXKNPXLTRMLTFKFYMPKKATELKHLQCLEEELKPLEEVLNLAXSKNFHLRPRDLISNINVIVLELKGSETTFMCEYADETATIVEFLNRWITFSQSIISTLT

SEQ ID NO: 6 - protein, 132 aa, synthetic construct
  VARIANT 4: note = nonnatural amino acid
  VARIANT 30: note = nonnatural amino acid
  VARIANT 34: note = nonnatural amino acid
  VARIANT 73: note = nonnatural amino acid
PTSXSTKKTQLQLEHLLLDLQMILNGINNXKNPXLTRMLTFKFYMPKKATELKHLQCLEEELKPLEEVLNLAXSKNFHLRPRDLISNINVIVLELKGSETTFMCEYADETATIVEFLNRWITFSQSIISTLT

SEQ ID NO: 7 - protein, 134 aa, synthetic construct
  SITE 6: note = para-azidomethyl-L-phenylalanine
  SITE 32: note = para-azidomethyl-L-phenylalanine
  SITE 36: note = para-azidomethyl-L-phenylalanine
  SITE 75: note = para-azidomethyl-L-phenylalanine
MAPTSFSTKKTQLQLEHLLLDLQMILNGINNFKNPFLTRMLTFKFYMPKKATELKHLQCLEEELKPLEEVLNLAFSKNFHLRPRDLISNINVIVLELKGSETTFMCEYADETATIVEFLNRWITFSQSIISTLT

SEQ ID NO: 8 - protein, 133 aa, synthetic construct
  SITE 5: note = para-azidomethyl-L-phenylalanine
  SITE 31: note = para-azidomethyl-L-phenylalanine
  SITE 35: note = para-azidomethyl-L-phenylalanine
  SITE 74: note = para-azidomethyl-L-phenylalanine
MPTSFSTKKTQLQLEHLLLDLQMILNGINNFKNPFLTRMLTFKFYMPKKATELKHLQCLEEELKPLEEVLNLAFSKNFHLRPRDLISNINVIVLELKGSETTFMCEYADETATIVEFLNRWITFSQSIISTLT

SEQ ID NO: 9 - protein, 132 aa, synthetic construct
  SITE 4: note = para-azidomethyl-L-phenylalanine
  SITE 30: note = para-azidomethyl-L-phenylalanine
  SITE 34: note = para-azidomethyl-L-phenylalanine
  SITE 73: note = para-azidomethyl-L-phenylalanine
PTSFSTKKTQLQLEHLLLDLQMILNGINNFKNPFLTRMLTFKFYMPKKATELKHLQCLEEELKPLEEVLNLAFSKNFHLRPRDLISNINVIVLELKGSETTFMCEYADETATIVEFLNRWITFSQSIISTLT

SEQ ID NO: 10 - protein, 134 aa, synthetic construct; identical to SEQ ID NO: 7
  SITE 6: note = para-azidomethyl-L-phenylalanine conjugated to nonpeptide, water-soluble polymer via a releasable linker
  SITE 32: note = para-azidomethyl-L-phenylalanine conjugated to nonpeptide, water-soluble polymer via a releasable linker
  SITE 36: note = para-azidomethyl-L-phenylalanine conjugated to nonpeptide, water-soluble polymer via a releasable linker
  SITE 75: note = para-azidomethyl-L-phenylalanine conjugated to nonpeptide, water-soluble polymer via a releasable linker

SEQ ID NO: 11 - protein, 133 aa, synthetic construct; identical to SEQ ID NO: 8
  SITE 5: note = para-azidomethyl-L-phenylalanine conjugated to nonpeptide, water-soluble polymer via a releasable linker
  SITE 31: note = para-azidomethyl-L-phenylalanine conjugated to nonpeptide, water-soluble polymer via a releasable linker
  SITE 35: note = para-azidomethyl-L-phenylalanine conjugated to nonpeptide, water-soluble polymer via a releasable linker
  SITE 74: note = para-azidomethyl-L-phenylalanine conjugated to nonpeptide, water-soluble polymer via a releasable linker

SEQ ID NO: 12 - protein, 132 aa, synthetic construct; identical to SEQ ID NO: 9
  SITE 4: note = para-azidomethyl-L-phenylalanine conjugated to nonpeptide, water-soluble polymer via a releasable linker
  SITE 30: note = para-azidomethyl-L-phenylalanine conjugated to nonpeptide, water-soluble polymer via a releasable linker
  SITE 34: note = para-azidomethyl-L-phenylalanine conjugated to nonpeptide, water-soluble polymer via a releasable linker
  SITE 73: note = para-azidomethyl-L-phenylalanine conjugated to nonpeptide, water-soluble polymer via a releasable linker

SEQ ID NO: 13 - protein, 134 aa, synthetic construct; identical to SEQ ID NO: 7
  SITE 6: note = para-azidomethyl-L-phenylalanine conjugated to PEG via a releasable linker
  SITE 32: note = para-azidomethyl-L-phenylalanine conjugated to PEG via a releasable linker
  SITE 36: note = para-azidomethyl-L-phenylalanine conjugated to PEG via a releasable linker
  SITE 75: note = para-azidomethyl-L-phenylalanine conjugated to PEG via a releasable linker

SEQ ID NO: 14 - protein, 133 aa, synthetic construct; identical to SEQ ID NO: 8
  SITE 5: note = para-azidomethyl-L-phenylalanine conjugated to PEG via a releasable linker
  SITE 31: note = para-azidomethyl-L-phenylalanine conjugated to PEG via a releasable linker
  SITE 35: note = para-azidomethyl-L-phenylalanine conjugated to PEG via a releasable linker
  SITE 74: note = para-azidomethyl-L-phenylalanine conjugated to PEG via a releasable linker

SEQ ID NO: 15 - protein, 132 aa, synthetic construct; identical to SEQ ID NO: 9
  SITE 4: note = para-azidomethyl-L-phenylalanine conjugated to PEG via a releasable linker
  SITE 30: note = para-azidomethyl-L-phenylalanine conjugated to PEG via a releasable linker
  SITE 34: note = para-azidomethyl-L-phenylalanine conjugated to PEG via a releasable linker
  SITE 73: note = para-azidomethyl-L-phenylalanine conjugated to PEG via a releasable linker

SEQ ID NO: 16 - protein, 134 aa, synthetic construct; identical to SEQ ID NO: 7
  SITE 6: note = para-azidomethyl-L-phenylalanine conjugated to SC579
  SITE 32: note = para-azidomethyl-L-phenylalanine conjugated to SC579
  SITE 36: note = para-azidomethyl-L-phenylalanine conjugated to SC579
  SITE 75: note = para-azidomethyl-L-phenylalanine conjugated to SC579

SEQ ID NO: 17 - protein, 133 aa, synthetic construct; identical to SEQ ID NO: 8
  SITE 5: note = para-azidomethyl-L-phenylalanine conjugated to SC579
  SITE 31: note = para-azidomethyl-L-phenylalanine conjugated to SC579
  SITE 35: note = para-azidomethyl-L-phenylalanine conjugated to SC579
  SITE 74: note = para-azidomethyl-L-phenylalanine conjugated to SC579

SEQ ID NO: 18 - protein, 132 aa, synthetic construct; identical to SEQ ID NO: 9
  SITE 4: note = para-azidomethyl-L-phenylalanine conjugated to SC579
  SITE 30: note = para-azidomethyl-L-phenylalanine conjugated to SC579
  SITE 34: note = para-azidomethyl-L-phenylalanine conjugated to SC579
  SITE 73: note = para-azidomethyl-L-phenylalanine conjugated to SC579

SEQ ID NO: 19 - protein, 142 aa, synthetic construct
MPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 20 - protein, 142 aa, synthetic construct
  SITE 5: note = para-azidomethylphenylalanine
MPTSFSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 21 - protein, 142 aa, synthetic construct
  SITE 31: note = para-azidomethylphenylalanine
MPTSSSTKKTQLQLEHLLLDLQMILNGINNFKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 22 - protein, 142 aa, synthetic construct
  SITE 35: note = para-azidomethylphenylalanine
MPTSSSTKKTQLQLEHLLLDLQMILNGINNYKNPFLTRMLTFKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 23 - protein, 142 aa, synthetic construct; identical to SEQ ID NO: 19
  SITE 42: note = para-azidomethylphenylalanine

SEQ ID NO: 24 - protein, 142 aa, synthetic construct
  SITE 74: note = para-azidomethylphenylalanine
MPTSSSTKKTQLQLEHLLLDLQMILNGINNYKNPKLTRMLTFKFYMPKKATELKHLQCLEEELKPLEEVLNLAFSKNFHLRPRDLISNINVIVLELKGSETTFMCEYADETATIVEFLNRWITFSQSIISTLTGGSHHHHHH

SEQ ID NO: 25 - protein, 142 aa, synthetic construct; identical to SEQ ID NO: 19
  SITE 78: note = para-azidomethylphenylalanine

SEQ ID NO: 26 - protein, 142 aa, synthetic construct
  SITE 81: note = para-azidomethylphenylalanine
MPTSSSTKKTQLQLEHLLLDLQMILNGINNYKNPKLTRMLTFKFYMPKKATELKHLQCLEEELKPLEEVLNLAQSKNFHLFPRDLISNINVIVLELKGSETTFMCEYADETATIVEFLNRWITFSQSIISTLTGGSHHHHHH

SEQ ID NO: 27 - protein, 142 aa, synthetic construct
  SITE 115: note = para-azidomethylphenylalanine
MPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIFEFLNRWITFSQSIISTLTGGSHHHHHH

SEQ ID NO: 28 - protein, 133 aa, synthetic construct [sequence omitted]

SEQ ID NO: 29 - protein, 133 aa, synthetic construct
  SITE 35: note = para-azidomethylphenylalanine
  SITE 42: note = para-azidomethylphenylalanine
MPTSSSTKKTQLQLEHLLLDLQMILNGINNYKNPFLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 30 - protein, 133 aa, synthetic construct
  SITE 31: note = para-azidomethylphenylalanine
  SITE 35: note = para-azidomethylphenylalanine
  SITE 42: note = para-azidomethylphenylalanine
MPTSSSTKKTQLQLEHLLLDLQMILNGINNFKNPFLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 31 - protein, 133 aa, synthetic construct
  SITE 5: note = para-azidomethylphenylalanine
  SITE 31: note = para-azidomethylphenylalanine
  SITE 35: note = para-azidomethylphenylalanine
MPTSFSTKKTQLQLEHLLLDLQMILNGINNFKNPFLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 32 - protein, 133 aa, synthetic construct
  SITE 5: note = para-azidomethylphenylalanine
  SITE 31: note = para-azidomethylphenylalanine
  SITE 35: note = para-azidomethylphenylalanine
MPTSFSTKKTQLQLEHLLLDLQMILNGINNFKNPFLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - protein, 142 aa, synthetic construct
  SITE 5: note = para-azidomethylphenylalanine
  SITE 35: note = para-azidomethylphenylalanine
  SITE 78: note = para-azidomethylphenylalanine
MPTSFSTKKTQLQLEHLLLDLQMILNGINNYKNPFLTRMLTFKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 34 - protein, 142 aa, synthetic construct; identical to SEQ ID NO: 33
  SITE 5: note = para-azidomethylphenylalanine
  SITE 35: note = para-azidomethylphenylalanine
  SITE 78: note = para-azidomethylphenylalanine

SEQ ID NO: 35 - protein, 142 aa, synthetic construct
  SITE 5: note = para-azidomethylphenylalanine
  SITE 35: note = para-azidomethylphenylalanine
  SITE 115: note = para-azidomethylphenylalanine
MPTSFSTKKTQLQLEHLLLDLQMILNGINNYKNPFLTRMLTFKFYMPKKATELKHLQCLEEELKPLEEVLNLAQSKNFHLRPRDLISNINVIVLELKGSETTFMCEYADETATIFEFLNRWITFSQSIISTLTGGSHHHHHH

SEQ ID NO: 36 - protein, 142 aa, synthetic construct
  SITE 5: note = para-azidomethylphenylalanine
  SITE 31: note = para-azidomethylphenylalanine
  SITE 35: note = para-azidomethylphenylalanine
MPTSFSTKKTQLQLEHLLLDLQMILNGINNFKNPFLTRMLTKKFYMPKKATELKHLQCLEEELKPLEEVLNLAQSKNFHLRPRDLISNINVIVLELKGSETTFMCEYADETATIVEFLNRWITFSQSIISTLTGGSHHHHHH

SEQ ID NO: 37 - protein, 142 aa, synthetic construct
  SITE 5: note = para-azidomethylphenylalanine
  SITE 31: note = para-azidomethylphenylalanine
  SITE 35: note = para-azidomethylphenylalanine
  SITE 81: note = para-azidomethylphenylalanine
MPTSFSTKKTQLQLEHLLLDLQMILNGINNFKNPFLTRMLTFKFYMPKKATELKHLQCLEEELKPLEEVLNLAQSKNFHLFPRDLISNINVIVLELKGSETTFMCEYADETATIVEFLNRWITFSQSIISTLTGGSHHHHHH

SEQ ID NO: 38 - protein, 142 aa, synthetic construct
  SITE 5: note = para-azidomethylphenylalanine
  SITE 31: note = para-azidomethylphenylalanine
  SITE 35: note = para-azidomethylphenylalanine
  SITE 74: note = para-azidomethylphenylalanine
MPTSFSTKKTQLQLEHLLLDLQMILNGINNFKNPFLTRMLTFKFYMPKKATELKHLQCLEEELKPLEEVLNLAFSKNFHLRPRDLISNINVIVLELKGSETTFMCEYADETATIVEFLNRWITFSQSIISTLTGGSHHHHHH

SEQ ID NO: 39 - protein, 142 aa, synthetic construct
  SITE 5: note = para-azidomethylphenylalanine
  SITE 31: note = para-azidomethylphenylalanine
  SITE 35: note = para-azidomethylphenylalanine
  SITE 115: note = para-azidomethylphenylalanine
MPTSFSTKKTQLQLEHLLLDLQMILNGINNFKNPFLTRMLTFKFYMPKKATELKHLQCLEEELKPLEEVLNLAQSKNFHLRPRDLISNINVIVLELKGSETTFMCEYADETATIFEFLNRWITFSQSIISTLTGGSHHHHHH

SEQ ID NO: 40 - protein, 142 aa, synthetic construct
  SITE 5: note = para-azidomethylphenylalanine
  SITE 31: note = para-azidomethylphenylalanine
  SITE 35: note = para-azidomethylphenylalanine
  SITE 81: note = para-azidomethylphenylalanine
MPTSFSTKKTQLQLEHLLLDLQMILNGINNFKNPFLTRMLTKKFYMPKKATELKHLQCLEEELKPLEEVLNLAQSKNFHLFPRDLISNINVIVLELKGSETTFMCEYADETATIVEFLNRWITFSQSIISTLTGGSHHHHHH

SEQ ID NO: 41 - protein, 133 aa, synthetic construct
  SITE 5: note = para-azidomethylphenylalanine
  SITE 35: note = para-azidomethylphenylalanine
  SITE 115: note = para-azidomethylphenylalanine
MPTSFSTKKTQLQLEHLLLDLQMILNGINNYKNPFLTRMLTFKFYMPKKATELKHLQCLEEELKPLEEVLNLAQSKNFHLRPRDLISNINVIVLELKGSETTFMCEYADETATIFEFLNRWITFSQSIISTLT

SEQ ID NO: 42 - protein, 133 aa, synthetic construct; identical to SEQ ID NO: 8
  SITE 5: note = para-azidomethylphenylalanine
  SITE 31: note = para-azidomethylphenylalanine
  SITE 35: note = para-azidomethylphenylalanine
  SITE 74: note = para-azidomethylphenylalanine

SEQ ID NO: 43 - protein, 133 aa, synthetic construct
  SITE 5: note = para-azidomethylphenylalanine
  SITE 31: note = para-azidomethylphenylalanine
  SITE 35: note = para-azidomethylphenylalanine
  SITE 115: note = para-azidomethylphenylalanine
MPTSFSTKKTQLQLEHLLLDLQMILNGINNFKNPFLTRMLTFKFYMPKKATELKHLQCLEEELKPLEEVLNLAQSKNFHLRPRDLISNINVIVLELKGSETTFMCEYADETATIFEFLNRWITFSQSIISTLT

SEQ ID NO: 44 - protein, 6 aa, synthetic construct
HHHHHH

(19) United States
(12) Patent Application Publication
(10) Pub. No.: US 2026/0256927 A1
(43) Pub. Date: Sep. 3, 2026

(54) INTERLEUKIN-2 PRODRUGS

(71) Applicant: Merck Sharp & Dohme LLC, Rahway, NJ (US)

(72) Inventors: Zachary Z. Brown, Princeton, NJ (US); Jongrock Kong, Princeton, NJ (US); Songnian Lin, Holmdel, NJ (US); Aarron Willingham, Mountain View, CA (US); Cristina Abrahams, Burlingame, CA (US); Krishna Bajjuri, Union City, CA (US); Xiaofan Li, Palo Alto, CA (US); Ryan Stafford, Foster City, CA (US); Cuong Tran, San Francisco, CA (US); Miao Wen, San Francisco, CA (US); Alice Yam, Belmont, CA (US); Gang Yin, South San Francisco, CA (US)

(73) Assignee: Merck Sharp & Dohme LLC, Rahway, NJ (US)

(21) Appl. No.: 18/873,119

(22) PCT Filed: Jun. 12, 2023
(86) PCT No.: PCT/US2023/025012

§ 371 Date: Dec. 9, 2024

Related U.S. Application Data
(60) Provisional application No. 63/352,737, filed on Jun. 16, 2022.

(51) Int. Cl. A61K 47/60; A61K 38/20; A61P 35/00; C07K 16/28

(52) U.S. Cl. CPC A61K 47/60; A61K 38/2013; A61P 35/00; C07K 16/2818; C07K 16/2827

(57) ABSTRACT

Interleukin-2 (IL-2) prodrugs useful for treatment and prevention of cell proliferation and cancer in a patient are provided.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 31, 2022, is named 25394-WO-PCT_SL.XML and is 91, 736 bytes in size.

### BACKGROUND OF THE INVENTION

### (1) Field of the Invention

The present invention relates to Interleukin-2 (IL-2) prodrugs. The IL-2 prodrugs are useful for treatment and prevention of cell proliferation and cancer in a patient.

### (2) Description of Related Art

Interleukin-2 (IL-2) was identified in 1965 as a factor produced in leukocyte cultures which when transferred, induced leukocyte blast formation. The factor behind this activity, the second cytokine to be identified over four decades ago, was initially called T-cell growth factor (TCGF). TCGF was named IL-2 in 1979 and in 1983 the cDNA for IL-2 was cloned. The first approval for IL-2 as a treatment for cancer (metastatic melanoma) occurred merely 8 years later.

The IL-2 protein is a four alpha helix cytokine measuring 15.5 kDa. IL-2 is produced by a number of cell types including NK T cells, CD8 T cells, mast cells and dendritic cells, but the main producers of IL-2 are antigen stimulated helper (CD4) T cells.

The effects of IL-2 are mediated by a complex receptor system comprised of three protein subunits, IL-2Rα (CD25), IL-2Rβ (CD122) and the common gamma chain (γ/γ_{c}/CD132). CD25 binds IL-2 with low affinity (no signal transduction). CD122 and CD132 form an intermediate affinity dimeric receptor (Kd, 10⁻⁹ M) which is expressed on CD8 T cells and NK cells. CD25, CD122, and CD132 form the high affinity trimer receptor system (Kd, 10⁻¹¹ M) that binds IL-2 with high affinity and is expressed on regulatory T cells (T_{regs}), activated T cells and endothelial cells. Due to this differential affinity, IL-2Rαβγ_{c} expressing cells will preferentially bind IL-2. A high dose of IL-2 activates the IL-2Rβγ_{c} dimer, resulting in activation of the immune response. A high dose of IL-2 also activates the IL-2Rαβγ_{c} trimer on T_{regs}, which suppresses activation of the immune response and may lead to tolerance of tumor antigens.

Binding of IL-2 to either IL-2Rβγ_{c} or IL-2Rαβγ_{c} induces multiple signaling pathways and the transcription of target genes. These pathways include the Jak/Stat pathway, the MAPK pathway and the PI3K pathway. Through these pathways, this potent cytokine induces activation, proliferation and cytokine production and differentiation of CD4 and CD8 T cells, and the activation of NK cells to promote their cytolytic functions. In addition, IL-2 promotes the induction of regulatory T cells (T_{regs}) which are inhibitory to the immune response. Discovered in 1999, the T_{reg} component of IL-2 biology illustrated the effect of IL-2 in both promoting or contracting the inflammatory immune response against foreign invaders such as pathogens or cancer; and added a level of nuance to the understanding of how high IL-2 doses promoted anti-tumor immunity by affecting the function of CD8 T cells and NK cells.

### IL-2 in Cancer Immunotherapy

IL-2 was the first cytokine, and immunotherapy, to be used successfully to treat cancer. In 1992, aldesleukin, a non-glycosylated human recombinant IL-2 analog (des-alanyl-1, serine-125 human IL-2), was approved by the U.S. Food and Drug Administration (FDA) for the treatment of Renal Cell Carcinoma (RCC) and Metastatic Melanoma. In these settings, high dose aldesleukin led to approximately 10% complete responses, however with dose limiting toxicities. Due to the short half-life of IL-2 (about one hour in humans) treatment of patients with IL-2 requires administration of approximately 3 mg by IV infusion over a 15-minute period every 8 hours for 14 doses over 5 days; following a 5 day break the course is repeated. An additional 1-2 courses of treatment might be given after 6-12 weeks.

Many patients treated with the high dose IL-2 regimen present with vascular leak syndrome (VLS) beginning 3-4 days after starting therapy; this effect was often dose limiting at days 5-10 of treatment, resulting in Intensive Care Unit admission. This syndrome is characterized by an increase in vascular permeability and extravasation of fluids and proteins from capillaries into tissues resulting in interstitial edema, decrease in organ perfusion and organ damage. Quantification of the most prominent Grade 3 and Grade 4 adverse events associated with IL-2 include hypotension and impaired renal function. Because administration of aldesleukin at the approved recommended doses can cause severe side effects, including VLS and impaired neutrophil function, FDA requires aldesleukin be marketed with a black box warning. Moreover, the commercial formulation of aldesleukin includes the presence of sodium dodecyl sulfate, a substance that appears to be required to maintain optimal activity through conformational stability. See Arakawa et al., 1994, Int. J. Peptide Protein Res. 43:583-587.

As the utility of IL-2 in the clinic has been hampered by its short half-life and by dose limiting toxicities, investigations have been concentrated towards mitigating these issues. Studies of the cellular and molecular mechanisms that result in VLS have implicated the interaction of IL-2 with CD25, the IL-2Rα chain, as the cause of VLS. Data supporting this hypothesis demonstrates that either half-life extended mutants of IL-2 that fail to interact with IL-2Rα or antibodies that block the IL-2/IL-2Rα binding interaction and confer prolonged half-life, provide efficacy in mouse models of cancer without inducing vascular leak. To confirm this effect using genetic deletions, bone marrow chimeras using mice harboring a deletion of CD25 in either only the immune system or only in the non-immune tissues found that the effect of IL-2 required only CD25 to be present in the non-immune tissues. These studies demonstrated the possibility of engineering a half-life extended IL-2 molecule that lacks the toxicity driving features while retaining the anti-neoplastic activity of wild-type IL-2.

After decades of attempts at engineering IL-2 molecules, positive data has emerged from the clinic. Progress in producing a half-life extended, biased, low-dose IL-2 has seemingly been accomplished by Nektar Therapeutics with a pegylated pro-drug form of aldesleukin called bempegaldesleukin (NKTR-214; See U.S. Pat. No. 9,861,705). Bempegaldesleukin has about 6 of its 11 lysine residues conjugated to hydrolysable bi-10 kDa polyethylene glycol (PEG) molecules such as to form an inactive prodrug. After 4 of the 6 PEG moieties are hydrolyzed, the bempegaldesleukin gains activity, with one or two of the remaining PEGs putatively positioned in a manner that biases binding of the molecule away from the IL-2Rα. Pegylation also endows bempegaldesleukin with a greatly increased half-life compared to wild-type native IL-2 (days compared to minutes) with prolonged exposure.

In addition to the treatment of proliferative diseases and disorders, IL-2 also has been suggested for the treatment of hepatitis C virus (HCV) infection, human immunodeficiency virus (HIV) infection, acute myeloid leukemia, non-Hodgkin's lymphoma, cutaneous T-cell lymphoma, juvenile rheumatoid arthritis, atopic dermatitis, breast cancer, and bladder cancer.

In light of the toxicity of aldesleukin and its relatively short half-life, there is a need for IL-2 analogs with reduced toxicity and extended half-life. Unmet improvements include stability, selectivity for instance at the various IL-2 receptor forms, dosing regimens, and limiting side effects. IL-2 muteins and conjugates may provide improved therapeutics for treating malignant melanoma, renal cell cancer, and other conditions receptive to IL-2 therapy.

### BRIEF SUMMARY OF THE INVENTION

The present invention provides an interleukin-2 (IL-2) prodrug comprising an IL-2 conjugate that comprises three or four nonnatural amino acids (NNAAs) conjugated to a releasable linker-polymer complex comprising a degradable linkage that when degraded releases the polymer from the IL-2 conjugate. The NNAAs are located in positions in an IL-2 polypeptide such that (a) when conjugated to releasable linker-polymer complexes comprising a degradable linkage, the IL-2 conjugate displays undetectable or significantly attenuated binding to the IL-2α receptor (IL-2Rα) and IL-2β (IL-2Rβ) receptor and further displays undetectable or significantly attenuated activity at the IL-2Rαβγ and/or IL-2Rβγ signaling complexes, both as determined by surface plasmon resonance compared to binding of an IL-2 moiety comprising the same NNAA substitutions but not conjugated to the releasable linker-polymer complex comprising a degradable linkage and (b) following release of the releasable linker-polymer complex from the IL-2 conjugate, a second IL-2 conjugate is formed, which can bind the IL-2Rα and IL-2Rβ and display activity at the IL-2Rαβγ and/or IL-2Rβγ signaling complexes. The second IL-2 conjugate at each conjugation site of the IL-2 conjugate comprises the portion of the releasable linker-polymer complex between the NNAA and the degradable linkage, which is herein referred to as a “stump”.

In an embodiment of the present invention, an IL-2 conjugate is provided comprising an IL-2 polypeptide comprising an amino acid sequence with at least 80% identity to the amino acid sequence set forth in SEQ ID NO: 2 or SEQ ID NO: 3, wherein the amino acids in at least three positions (and in specific embodiments, three or four positions) of the IL-2 polypeptide are each substituted with a NNAA conjugated to a nonpeptidic, water-soluble polymer by a releasable linker comprising a degradable linkage, wherein the IL-2 conjugate displays undetectable or significantly attenuated binding to the IL-2α receptor (IL-2Rα) and IL-2β (IL-2Rβ) receptor and further displays undetectable or significantly attenuated activity at the IL-2Rαβγ or IL-2Rβγ signaling complex, both as determined by surface plasmon resonance compared to binding of an IL-2 moiety comprising the same NNAA substitutions and becomes capable of binding to the IL-2Rα and IL-2Rβ and displaying activity at the IL-2Rαβγ or IL-2Rβγ signaling complex following release of the releasable linker-polymer complex from the IL-2 conjugate.

In an embodiment of the present invention, an IL-2 conjugate is provided comprising an IL-2 polypeptide comprising an amino acid sequence with at least 80% identity to the amino acid sequence set forth in SEQ ID NO: 2 or SEQ ID NO: 3, wherein the amino acids in at least three of positions S4, Y30, K34, Q73, and V114 of the IL-2 polypeptide in reference to the amino acid positions within SEQ ID NO: 2 are each substituted with a NNAA conjugated to a nonpeptidic, water-soluble polymer by a releasable linker comprising a degradable linkage.

In particular embodiments, substitution of amino acids at positions other than S4, Y30, K34, Q73, and V114 may be natural amino acids which do not decrease or abrogate binding to the IL-2α and IL-2 β receptors.

In particular embodiments, the IL-2 polypeptide comprises an amino acid sequence with at least 85% identity to the amino acid sequence set forth in SEQ ID NO: 2 or SEQ ID NO: 3. In particular embodiments, the IL-2 polypeptide comprises an amino acid sequence with at least 90% identity to the amino acid sequence set forth in SEQ ID NO: 2 or SEQ ID NO: 3. In particular embodiments, the IL-2 polypeptide comprises an amino acid sequence with at least 95% identity to the amino acid sequence set forth in SEQ ID NO: 2 or SEQ ID NO: 3. In further embodiments, the IL-2 polypeptide further includes a substitution of the cysteine residue at position 124 with an amino acid selected from the group consisting of alanine and serine.

In particular embodiments, the IL-2 polypeptide comprises an amino acid sequence with at least 90% identity to the amino acid sequence set forth in SEQ ID NO: 3. In particular embodiments, the IL-2 polypeptide comprises an amino acid sequence with at least 95% identity to the amino acid sequence set forth in SEQ ID NO: 3.

In further embodiments of the IL-2 conjugate, each of the amino acids at positions S4, Y30, K34, and Q73 are substituted with a NNAA conjugated to a nonpeptidic, water-soluble polymer by a releasable linker comprising a degradable linkage.

In particular embodiments of the IL-2 conjugate, the IL-2 polypeptide comprises an amino acid sequence with at least 85% identity to the amino acid sequence set forth in SEQ ID NO: 3 and each of the amino acids at positions S4, Y30, K34, and Q73 are substituted with a NNAA conjugated to a nonpeptidic, water-soluble polymer by a releasable linker comprising a degradable linkage. In particular embodiments of the IL-2 conjugate, the IL-2 polypeptide comprises an amino acid sequence with at least 90% identity to the amino acid sequence set forth in SEQ ID NO: 3 and each of the amino acids at positions S4, Y30, K34, and Q73 are substituted with a NNAA conjugated to a nonpeptidic, water-soluble polymer by a releasable linker comprising a degradable linkage. In particular embodiments of the IL-2 conjugate, the IL-2 polypeptide comprises an amino acid sequence with at least 95% identity to the amino acid sequence set forth in SEQ ID NO: 3 and each of the amino acids at positions S4, Y30, K34, and Q73 are substituted with a NNAA conjugated to a nonpeptidic, water-soluble polymer by a releasable linker comprising a degradable linkage.

In the forementioned embodiments in which the amino acid sequence has 80%, 85%, 90%, or 95% identity to the amino acid sequence set forth in SEQ ID NO: 3, the amino acids that differ from the amino acid sequence set forth in SEQ ID NO: 3 are not in positions that reduce the binding of the embodiment to the IL-2α or IL-2β receptor compared to an IL-2 moiety comprising the amino acid sequence of SEQ ID NO: 9 as determined by surface plasmon resonance.

In particular embodiments, the IL-2 polypeptide comprises the amino acid sequence set forth in SEQ ID NO: 3 wherein the amino acids at positions S4, Y30, K34, and Q73 of the IL-2 polypeptide in reference to the amino acid positions within SEQ ID NO: 3 are each substituted with a NNAA conjugated to a nonpeptidic, water-soluble polymer by a releasable linker comprising a degradable linkage.

In further embodiments, the NNAA comprises a functional group and the releasable linker comprises a reactive group that reacts with the functional group to form a covalent linkage between the functional group of the NNAA and the reactive group of the releasable linker.

In further embodiments, the NNAA is selected from the group consisting of p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine.

In further embodiments, the non-natural amino acid residues are selected from a compound of Formula (XXXI):

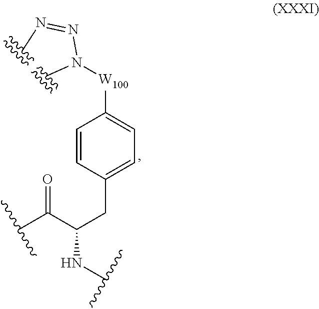

wherein W₁₀₀ is C₁₋₁₀ alkylene, wherein the double wavy lines indicate attachment to a moiety of the releasable linker, and wherein the wavy lines indicate attachment to adjacent amino acids in the IL-2 polypeptide. In a further embodiment, W₁₀₀ is a C₁₋₃ alkylene. In further embodiments, the NNAA is p-azidomethyl-L-phenylalanine.

In further embodiments, the nonpeptidic, water-soluble polymer has an average molecular weight between about 5 kDa and about 50 kDa. In further embodiments, the nonpeptidic, water-soluble polymer has an average molecular weight of about 20 kDa.

In further embodiments, the nonpeptidic, water-soluble polymer is polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. In further embodiments, the nonpeptidic, water-soluble polymer comprises a linear or branched PEG or linear or branched mPEG.

In further embodiments, the releasable linker comprises a fluorenylmethyloxycarbonyl (Fmoc) group covalently linked to the nonpeptidic, water-soluble polymer and to the reactive group to provide a releasable linker comprising a degradable linkage.

In a further embodiment, the releasable linker is selected from a compound of Formula (XXXII):

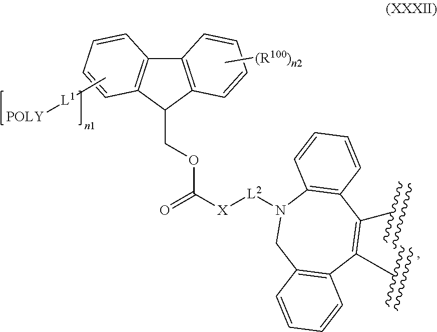

wherein the double wavy lines indicate attachment to a moiety of the non-natural amino acid residue; POLY is a nonpeptidic, water-soluble polymer; L¹ and L² are independently selected from the group consisting of —O—C₁-C₆alkylene-NH—C(O)—C₁-C₆alkylene-C(O)—NH—, —O—C₁-C₆alkylene-NH—C(O)—C₁-C₆alkylene-, —O—C₁-C₆alkylene-NH—C(O)—C₁-C₆alkylene-C(O)—NH—C₁-C₆alkylene-, —O—C₁-C₆alkylene-NH—C(O)—, and —C₁-C₆alkylene-C(O)—, wherein each —C₁-C₆alkylene- is independently optionally substituted with one or more substituents independently selected from halogen, alkyl, haloalkyl, hydroxyl, amino, alkylamino, and alkoxy;
X is a bond, —O—, or —N(R²)—; each R¹⁰⁰ is hydrogen or lower alkyl; n1 is an integer selected from one to two; and n2 is an integer selected from one to four. In a further embodiment, the L¹ is —O—C₁-C₆alkylene-NH—C(O)—. In a further embodiment, L² is —C₁-C₆alkylene-C(O)—. In a further embodiment, the L¹ is —O—C₁-C₆alkylene-NH—C(O)— and L² is —C₁-C₆alkylene-C(O)—. In further embodiments, n1 is 1. In further embodiments, R¹⁰⁰ is hydrogen. In a further still embodiment, n2 is 1. In further still embodiments, n1 and n2 are each 1 and R¹⁰⁰ is hydrogen.

In further embodiments, the releasable linker comprises a distal end and a proximal end, wherein the proximal end comprises a dibenzocyclooctyne (DBCO)amine and the distal end comprises a 9-fluorenylmethyloxycarbonyl (Fmoc) directly or indirectly covalently linked to the nonpeptidic, water-soluble polymer, and wherein the linkage between the amino group of the DBCO amine and the methoxycarbonyl of the Fmoc comprises an ester, which provides the degradable linkage.

In further embodiments, the releasable linker comprises the formula

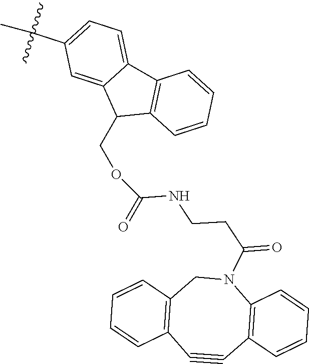 [SMILES: *c1ccc2c(c1)C(COC(=O)NCCC(=O)N1Cc3ccccc3C#Cc3ccccc31)c1ccccc1-2]

wherein the wavy line indicates a covalent bond between the Fmoc group and the nonpeptidic, water-soluble polymer.

In further embodiments, the covalent linkage between the functional group of the NNAA and the reactive group of the releasable linker comprises a triazole.

In further embodiments, the releasable linker conjugated to the nonpeptidic, water soluble polymer by a degradable linkage and covalently linked to each NNAA independently comprises the formula

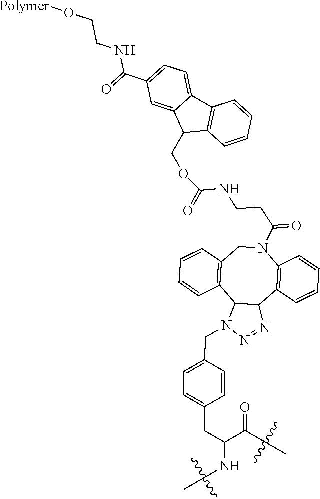

or its regioisomer

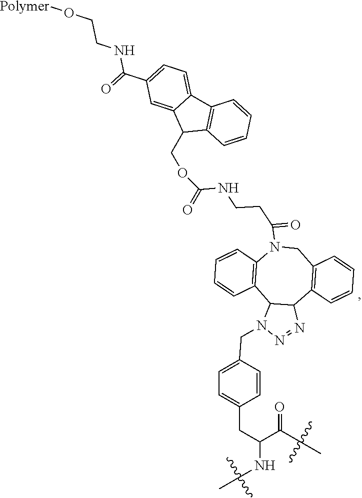

wherein polymer refers to a nonpeptidic, water-soluble polymer, and the wavy lines indicate covalent bonds to adjacent amino acids in the IL-2 polypeptide.

In further embodiments, degradation of a degradable linkage results in an NNAA conjugated to a DBCO amine stump comprising the formula

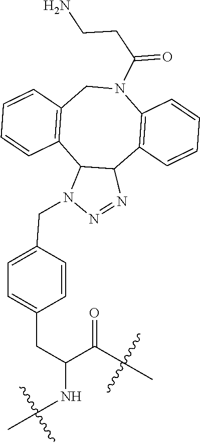 [SMILES: *NC(Cc1ccc(CN2N=NC3c4ccccc4N(C(=O)CCN)Cc4ccccc4C32)cc1)C(*)=O]

or its regioisomer

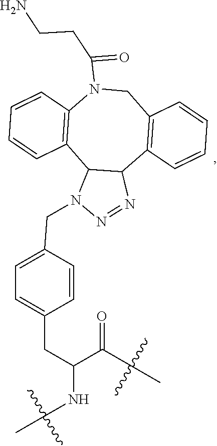 [SMILES: *NC(Cc1ccc(CN2N=NC3c4ccccc4CN(C(=O)CCN)c4ccccc4C32)cc1)C(*)=O]

wherein the wavy lines indicate covalent bonds to adjacent amino acids in the IL-2 polypeptide.

In further embodiments, degradation of each degradable linkage in the IL-2 conjugate produces a second IL-2 conjugate wherein each NNAA is conjugated to the DBCO amine stump.

In further embodiments, the amino acid set forth in SEQ ID NO: 2 or SEQ ID NO: 3 further comprises at the N-terminus a methionine residue, an alanine residue, or a methionine alanine dipeptide. In further embodiments, the IL-2 polypeptide comprises at the N-terminus a methionine residue, an alanine residue, or a methionine alanine dipeptide.

In further embodiments, the IL-2 conjugate comprises the amino acid sequence set forth in SEQ ID NO: 10, 11, or 12. In further embodiments, the IL-2 conjugate comprises the amino acid sequence set forth in SEQ ID NO: 13, 14, or 15. In further embodiments, the IL-2 conjugate comprises the amino acid sequence set forth in SEQ ID NO: 16, 17, or 18.

In further embodiments, the IL-2 conjugate has undetectable or significantly attenuated binding to the IL-2α and IL-2 β receptors when each NNAA is covalently linked to the nonpeptidic, water-soluble polymer by the releasable linker as determined by surface plasmon resonance when compared to binding of an IL-2 polypeptide not covalently linked to the nonpeptidic, water-soluble polymer.

In further embodiments, the second IL-2 conjugate has a binding affinity for the IL-2α and IL-2 β receptors of about 6×10⁻⁷ or less as determined by surface plasmon resonance.

The present invention further provides an interleukin 2 (IL-2) conjugate comprising an IL-2 polypeptide comprising an amino acid sequence with at least 80% identity to the amino acid sequence set forth in SEQ ID NO: 2 or SEQ ID NO: 3, wherein the IL-2 polypeptide comprises 3-6 non-natural amino acid residues, and wherein each of the 3-6 non-natural amino acid residues is site-specifically linked to a nonpeptidic, water-soluble polymer by a releaseable linker comprising a degradable linkage.

In a further embodiment, the IL-2 polypeptide comprises an amino acid sequence with at least 90% identity to the amino acid sequence set forth in SEQ ID NO: 2 or SEQ ID NO: 3. In a further embodiment, the IL-2 conjugate of claim 46 or 47, wherein the IL-2 polypeptide comprises 3-4 non-natural amino acid residues. In further embodiments, the IL-2 polypeptide comprises the non-natural amino acid residues at specific sites selected from the group consisting of: S4, Y30, K34, F41, Q73, F77, R80 and V114 relative to their positions shown in SEQ ID NO: 2. In a further embodiments, the IL-2 polypeptide comprises the non-natural amino acid residues at specific sites selected from the group consisting of: S4, Y30, K34, Q73, and V114, relative to SEQ ID NO: 2.

In further embodiments, the non-natural amino acid residues are selected from the group consisting of p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine.

In further embodiments, the non-natural amino acid residues are selected from a compound of Formula (XXXI):

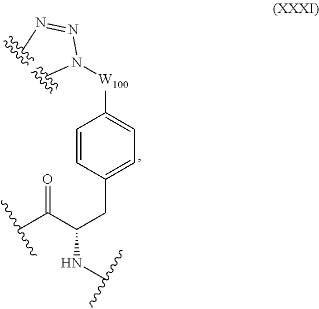

wherein W₁₀₀ is C₁₋₁₀ alkylene, wherein the double wavy lines indicate attachment to a moiety of the releasable linker, and wherein the wavy lines indicate covalent bonds to adjacent amino acids in the IL-2 polypeptide. In further embodiments, the W₁₀₀ is C₁₋₃ alkylene.

In further embodiments, the nonpeptidic, water-soluble polymer comprises polyethylene glycol (PEG). In further embodiments, the PEG has an average molecular weight of about 10 kDa to 20 kDa.

In further embodiments, releasable linker is selected from a compound of Formula (XXXII):

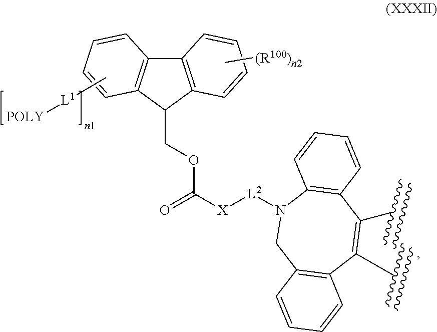

wherein the double wavy lines indicate attachment to a moiety of the non-natural amino acid residue; POLY is a nonpeptidic, water-soluble polymer; L¹ and L² are independently selected from the group consisting of —O—C₁-C₆alkylene-NH—C(O)—C₁-C₆alkylene-C(O)—NH—, —O—C₁-C₆alkylene-NH—C(O)—C₁-C₆alkylene-, —O—C₁-C₆alkylene-NH—C(O)—C₁-C₆alkylene-C(O)—NH—C₁-C₆alkylene-, —O—C₁-C₆alkylene-NH—C(O)—, and —C₁-C₆alkylene-C(O)—, wherein each —C₁-C₆alkylene- is independently optionally substituted with one or more substituents independently selected from halogen, alkyl, haloalkyl, hydroxyl, amino, alkylamino, and alkoxy;
X is a bond, —O—, or —N(R²)—; each R¹⁰⁰ is hydrogen or lower alkyl; n1 is an integer selected from one to two; and n2 is an integer selected from one to four. In a further embodiment, the L¹ is —O—C₁-C₆alkylene-NH—C(O)—. In a further embodiment, L² is —C₁-C₆alkylene-C(O)—. In a further embodiment, the L¹ is —O—C₁-C₆alkylene-NH—C(O)— and L² is —C₁-C₆alkylene-C(O)—. In further embodiments, n1 is 1. In further embodiments, R¹⁰⁰ is hydrogen. In a further still embodiment, n2 is 1. In further still embodiments, N1 and n2 are each 1 and R¹⁰⁰ is hydrogen.

The present invention further provides an IL-2 conjugate comprising an IL-2 polypeptide comprising an amino acid sequence having at least 80% identity to the amino acid sequence of SEQ ID NO: 2 or SEQ ID NO: 3 in which the amino acid residues in the IL-2 conjugate at amino acid positions S4, Y30, K34, and Q73 in reference to the amino acid positions within SEQ ID NO: 2, are each replaced by the structure of Formula (I):

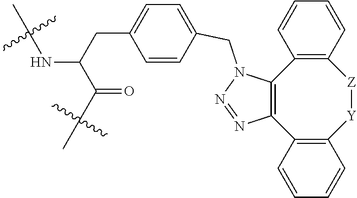

wherein:
Z is CH₂ and Y is

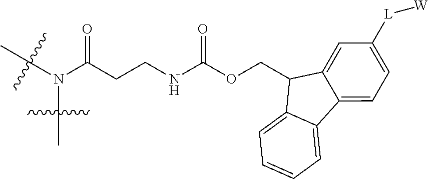

or
Y is CH₂ and Z is

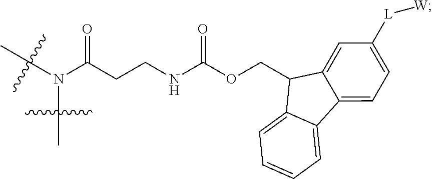

L comprises a spacer moiety; and
W comprises a nonpeptidic, water-soluble polymer.

In particular embodiments, the IL-2 polypeptide comprises an amino acid sequence with at least 85% identity to the amino acid sequence set forth in SEQ ID NO: 2 or SEQ ID NO: 3. In particular embodiments, the IL-2 polypeptide comprises an amino acid sequence with at least 90% identity to the amino acid sequence set forth in SEQ ID NO: 2 or SEQ ID NO: 3. In particular embodiments, the IL-2 polypeptide comprises an amino acid sequence with at least 95% identity to the amino acid sequence set forth in SEQ ID NO: 2 or SEQ ID NO: 3.

In particular embodiments, the IL-2 polypeptide comprises the amino acid sequence set forth in SEQ ID NO: 3 wherein the amino acids at positions S4, Y30, K34, and Q73 of the IL-2 polypeptide in reference to the amino acid positions within SEQ ID NO: 3 are each replaced with the aforementioned structure.

In further embodiments, the IL-2 polypeptide further includes a substitution of the cysteine residue at position 124 with an amino acid selected from the group consisting of alanine and serine. In particular embodiments, the IL-2 polypeptide comprises an amino acid sequence with at least 90% identity to the amino acid sequence set forth in SEQ ID NO: 3. In particular embodiments, the IL-2 polypeptide comprises an amino acid sequence with at least 95% identity to the amino acid sequence set forth in SEQ ID NO: 3.

In further embodiments, the nonpeptidic, water-soluble polymer comprises polyethylene glycol (PEG) or methoxypolyethylene glycol (mPEG). In further embodiments, the PEG or mPEG has an average molecular weight of 20 kDa. In particular embodiments, the PEG or mPEG comprises an average of about 454 ethylene glycol units.

In further embodiments, L comprises a covalent bond or a C₁-C₁₀ alkyl or substituted alkyl. In further embodiments, L-W comprises the formula

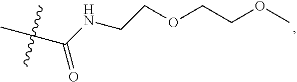

wherein n is the number of ethylene glycol units sufficient to provide an mPEG having an average molecular weight of 20 kDa. In particular embodiments, n is about 454.

The present invention further provides an IL-2 conjugate, which comprises the amino acid sequence set forth in any one of any of SEQ ID Nos: 10, 11, or 12, wherein the p-azidomethyl-L-phenylalanine conjugated to nonpeptide, water-soluble polymer via a releasable linker at each position independently has the formula selected from the group consisting of:

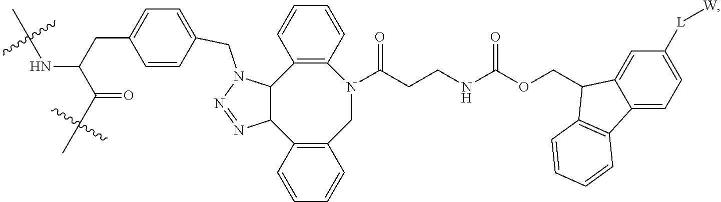

and its regioisomer

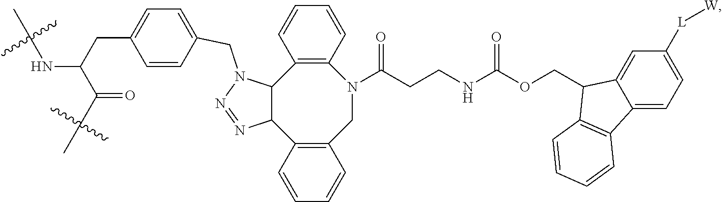

wherein L comprises a spacer moiety; and W comprises a nonpeptidic, water-soluble polymer.

The present invention further provides an IL-2 conjugate, which comprises the amino acid sequence set forth in any one of any of SEQ ID Nos: 13, 14, or 15, wherein the p-azidomethyl-L-phenylalanine conjugated to polyethylene glycol (PEG) or methoxypolyethylene glycol (mPEG) via a releasable linker at each position independently has the formula selected from the group consisting of:

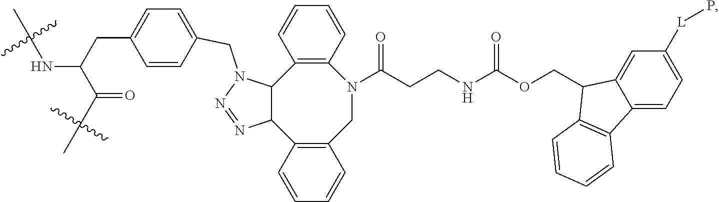

and its regioisomer

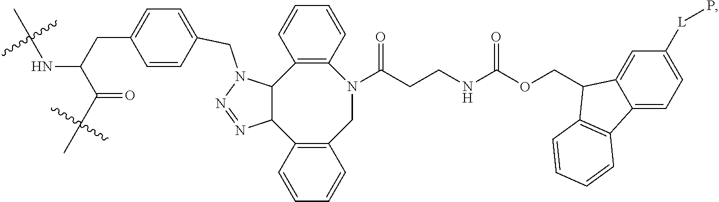

wherein L comprises a spacer moiety; and P comprises PEG or mPEG.

The present invention further provides an IL-2 conjugate, which comprises the amino acid sequence set forth in any one of any of SEQ ID Nos: 16, 17, or 18, wherein the p-azidomethyl-L-phenylalanine conjugated to SC579 via a releasable linker at each position independently has the formula selected from the group consisting of:

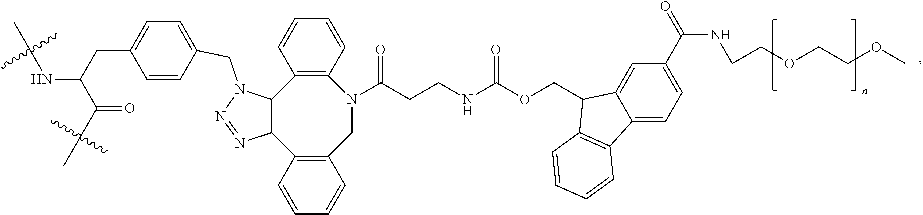

and its regioisomer

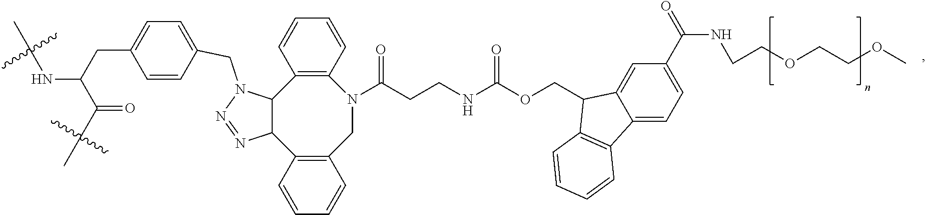

wherein n is the number of ethylene glycol units sufficient to provide an mPEG having an average molecular weight of 20 kDa.

The present invention further provides a composition comprising the IL-2 conjugate of any one of the foregoing embodiments and a pharmaceutically acceptable carrier or excipient.

The present invention further provides a method of treating or preventing a disease or condition in a subject in need thereof, comprising administering to the subject an effective amount of the IL-2 conjugate of any one of the foregoing embodiments or a composition comprising the IL-2 conjugate of any one of the foregoing embodiments and a pharmaceutically acceptable carrier or excipient.

The present invention further provides a method for treating a proliferative disease or cancer in an individual, comprising administering a therapeutically effective amount of the IL-2 conjugate of any one of the foregoing embodiments or the foregoing composition to an individual in need thereof to treat the proliferative disease or cancer in the individual.

The present invention further provides a combination therapy for treating a proliferative disease or cancer in an individual, comprising administering a therapeutically effective amount of the IL-2 conjugate of any one of the foregoing embodiments or the foregoing composition to an individual in need thereof, and administering a therapeutically effective amount of a therapeutic agent to the individual, to treat the proliferative disease or cancer in the individual. In particular embodiments, the therapeutic agent is an anti-PD1 antibody or anti-PDL1 antibody.

In particular embodiments, the IL-2 conjugate or composition is administered before the therapeutic agent is administered; wherein the IL-2 conjugate or composition is administered after the therapeutic agent is administered, or wherein the IL-2 conjugate or composition is administered concurrently with the therapeutic agent.

The present invention further provides an IL-2 conjugate or composition disclosed herein for the treatment of a proliferative disease or cancer.

The present invention further provides an IL-2 conjugate or composition disclosed herein for the manufacture of a medicament for the treatment of a proliferative disease or cancer.

The present invention further provides an IL-2 variant comprising the amino acid sequence set forth in SEQ ID NO: 4, SEQ ID NO: 5, or SEQ ID NO: 6. In particular embodiments, the nonnatural amino acid (NNAA) is selected from the group consisting of: p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine. In a further embodiment, the NNA comprises p-azidomethyl-L-phenylalanine.

The present invention further provides an IL-2 variant comprising the amino acid sequence set forth in SEQ ID NO: 7, SEQ ID NO: 8, or SEQ ID NO: 9.

The present invention further provides for the use of any of the foregoing IL-2 variants for the manufacture of a medicament for treating a proliferative disease or cancer.

The present invention further provides an IL-2 conjugate comprising an IL-2 polypeptide comprising an amino acid sequence with at least 80% identity to the amino acid sequence set forth in SEQ ID NO: 2 or SEQ ID NO: 3,
wherein the amino acids at positions S4, Y30, K34, and Q73 of the IL-2 polypeptide in reference to the amino acid positions within SEQ ID NO: 3 are each substituted with a para-azidomethylphenylalanine (pAMF) conjugated to a dibenzocyclooctyne (DBCO) amine.

In particular embodiments, the IL-2 polypeptide comprises an amino acid sequence with at least 85% identity to the amino acid sequence set forth in SEQ ID NO: 2 or SEQ ID NO: 3. In particular embodiments, the IL-2 polypeptide comprises an amino acid sequence with at least 90% identity to the amino acid sequence set forth in SEQ ID NO: 2 or SEQ ID NO: 3. In particular embodiments, the IL-2 polypeptide comprises an amino acid sequence with at least 95% identity to the amino acid sequence set forth in SEQ ID NO: 2 or SEQ ID NO: 3.

In particular embodiments, the IL-2 polypeptide comprises an amino acid sequence with at least 80%, 85%, 90%, 95%, or 100% identity to the amino acid sequence set forth in SEQ ID NO: 2 or SEQ ID NO: 3, wherein three or four of the amino acids at positions S4, Y30, K34, Q73, and V114 of the IL-2 polypeptide in reference to the amino acid positions within SEQ ID NO: 2 or SEQ ID NO: 3 are each substituted with a para-azidomethylphenylalanine (pAMF) conjugated to a dibenzocyclooctyne (DBCO)amine.

In particular embodiments, the IL-2 polypeptide comprises an amino acid sequence with at least 80%, 85%, 90%, 95%, or 100% identity to the amino acid sequence set forth in SEQ ID NO: 2 or SEQ ID NO: 3, wherein four of the amino acids at positions S4, Y30, K34, Q73, and V114 of the IL-2 polypeptide in reference to the amino acid positions within SEQ ID NO: 2 or SEQ ID NO: 3 are each substituted with a para-azidomethylphenylalanine (pAMF) conjugated to a dibenzocyclooctyne (DBCO)amine.

In particular embodiments, the IL-2 polypeptide comprises an amino acid sequence with at least 80%, 85%, 90%, 95%, or 100% identity to the amino acid sequence set forth in SEQ ID NO: 2 or SEQ ID NO: 3, wherein the amino acids at positions S4, Y30, K34, and Q73 of the IL-2 polypeptide in reference to the amino acid positions within SEQ ID NO: 2 or SEQ ID NO: 3 are each substituted with a para-azidomethylphenylalanine (pAMF) conjugated to a dibenzocyclooctyne (DBCO)amine.

In particular embodiments, the IL-2 polypeptide comprises an amino acid sequence with at least 80%, 85%, 90%, 95%, or 100% identity to the amino acid sequence set forth in SEQ ID NO: 2 or SEQ ID NO: 3, wherein the amino acids at positions S4, Y30, K34, and V114 of the IL-2 polypeptide in reference to the amino acid positions within SEQ ID NO: 2 or SEQ ID NO: 3 are each substituted with a para-azidomethylphenylalanine (pAMF) conjugated to a dibenzocyclooctyne (DBCO)amine.

In particular embodiments of the above, the pAMF conjugated to the DBCO amine at each position has a formula independently selected from the group consisting of:

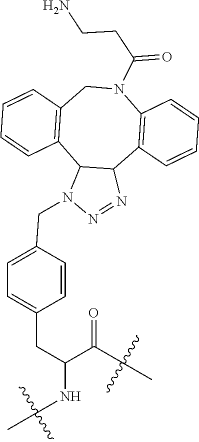 [SMILES: *NC(Cc1ccc(CN2N=NC3c4ccccc4N(C(=O)CCN)Cc4ccccc4C32)cc1)C(*)=O]

or its regioisomer

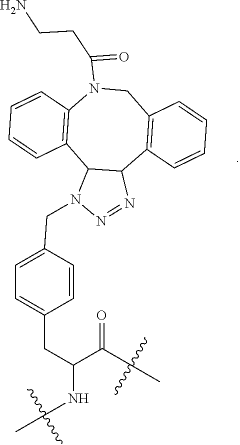 [SMILES: *NC(Cc1ccc(CN2N=NC3c4ccccc4CN(C(=O)CCN)c4ccccc4C32)cc1)C(*)=O]

### BRIEF DESCRIPTION OF THE DRAWINGS

FIG. 1A shows the chemistry on conjugation of releasable linker-polymer complexes comprising degradable linkages to an IL-2 moiety comprising four NNAAs to produce an IL-2 prodrug and the release of the releasable linker-polymer complex from the IL-2 prodrug over time by degradation of the degradable linkage. As exemplified, releasable linker-polymer complexes comprising a degradable linkage are conjugated to an IL-2 moiety comprising four para-azidomethylphenylalanine (pAMF) residues to produce IL-2 prodrugs comprising IL-2 conjugates in which each pAMF is conjugated to the releasable linker-polymer complex. Release of the releasable linker-polymer complex over time produces a second IL-2 conjugate comprising four “stump” residues comprising the portion of the releasable linker-polymer complex that remains conjugated to the IL-2 moiety following degradation of the degradable linkage.

FIG. 1B shows a model of the binding of a second IL-2 conjugate comprising four stumps at positions S4, Y30, K34, and Q73 to the IL-2 αβγ receptor complex.

FIG. 2A shows tumor growth curves in response to indicated dose of IL-2 PEG variants with different total PEG size and conjugation sites administered intravenously q7dx2 (once a week for two weeks) to animals bearing established B16F10 syngeneic mouse melanoma tumors. Statistical analysis was performed on tumor sizes at day 10 using one-way ANOVA with Dunnett's multiple comparison test. A probability of less than 5% (p less than 0.05) was considered as significant.

FIG. 2B shows percent body weight change in animals bearing syngeneic mouse melanoma tumor model B16F10 in response to indicated dose of IL-2 PEG analog variants administered q7dx2. Percent body weight change was calculated relative to animal weight on the first day treatment was administered. Data are presented as mean values±SEM (n=9 per group).

FIGS. 3A and 3C show tumor growth curves in response to indicated dose of IL-2 PEG variants engineered with or without R37AF41K mutations administered intravenously q7dx3 (once a week for three weeks) to animals bearing established B16F10 syngeneic mouse melanoma tumors. Statistical analysis was performed on tumor sizes at day 10 using one-way ANOVA with Dunnett's multiple comparison test. A probability of less than 5% (p less than 0.05) was considered as significant.

FIGS. 3B and 3D show percent body weight change in animals bearing syngeneic mouse melanoma tumor model B16F10 in response to indicated dose of IL-2 PEG variants engineered with or without R37AF41K mutations administered q7dx3. Percent body weight change was calculated relative to animal weight on the first day treatment was administered. Data are presented as mean values±SEM (n=9 per group).

FIG. 4A shows tumor growth curves in response to indicated dose of IL-2 PEG analog variants administered intravenously q7dx2 to animals bearing established B16F10 syngeneic mouse melanoma tumors. Statistical analysis was performed on tumor sizes at day 10 using one-way ANOVA with Dunnett's multiple comparison test. A probability of less than 5% (p less than 0.05) was considered as significant. P-values: *=p less than 0.5, ns=not significant.

FIG. 4B shows percent body weight change in animals bearing syngeneic mouse melanoma tumor model B16F10 in response to indicated dose of IL-2 PEG analog variants administered q7dx2. Percent body weight change was calculated relative to animal weight on the first day treatment was administered. Data are presented as mean values±SEM (n=9 per group).

FIG. 5A shows tumor growth curves in response to increasing doses of SP10784 administered intravenously q7dx2 to animals bearing established B16F10 syngeneic mouse melanoma tumors. Statistical analysis was performed on tumor sizes at day 11 using one-way ANOVA with Dunnett's multiple comparison test. A probability of less than 5% (p less than 0.05) was considered as significant. P-values: *=p less than 0.5, **p less than 0.01, ****p less than 0.0001.

FIG. 5B shows percent body weight change in animals bearing syngeneic mouse melanoma tumor model B16F10 in response to increasing doses of SP10784 administered q7dx2. Percent body weight change was calculated relative to animal weight on the first day treatment was administered. Data are presented as mean values±SEM (n=9 per group).

FIGS. 6A-6H depict changes in the tumoral immune compartment following a single intravenous dose of 5 mg/kg SP10784 in animals bearing B16F10 tumors. Single-cell suspensions from B16F10 tumors collected on day 3, 7, and 10 post treatment were obtained using the Mouse Tumor Dissociation Kit from Miltenyi Biotec Inc. (cat: 130-096-730) and analysed via flow cytometry.

FIG. 6A depicts changes in the frequency of tumor-infiltrating natural killer (NK) cells following a single intravenous dose of 5 mg/kg SP10784, reported as a percentage of total live CD45+ cells as measured by flow cytometry. Data are presented as mean±SEM (n=2-4 per group).

FIG. 6B depicts changes in the proportion of granzyme-B-positive (GZMB⁺) NK cells following a single intravenous dose of 5 mg/kg SP10784, reported as a percentage of total NK cells as measured by flow cytometry. Data are presented as mean±SEM (n=2-4 per group).

FIG. 6C depicts changes in the frequency of tumor-infiltrating CD8⁺ T cells following a single intravenous dose of 5 mg/kg SP10784, reported as a percentage of total live CD45⁺ cells as measured by flow cytometry. Data are presented as mean±SEM (n=2-4 per group).

FIG. 6D depicts changes in the proportion of GZMB⁺ CD8⁺ T cells following a single intravenous dose of 5 mg/kg SP10784, reported as a percentage of total CD8⁺ T cells as measured by flow cytometry. Data are presented as mean±SEM (n=2-4 per group).

FIG. 6E depicts changes in the frequency of tumor-infiltrating CD4⁺ Thelper (Th) cells following a single intravenous dose of 5 mg/kg SP10784, reported as a percentage of total live CD45⁺ cells as measured by flow cytometry. Data is presented as mean±SEM (n=2-4 per group).

FIG. 6F depicts changes in the proportion of GZMB⁺ CD4⁺ Th cells following a single intravenous dose of 5 mg/kg SP10784, reported as a percentage of total CD4⁺ Th cells as measured by flow cytometry. Data are presented as mean±SEM (n=2-4 per group).

FIG. 6G depicts changes in the frequency of tumor-infiltrating CD4⁺ regulatory T cells (Tregs) following a single intravenous dose of 5 mg/kg SP10784, reported as a percentage of total live CD45⁺ cells as measured by flow cytometry. Data is presented as mean±SEM (n=2-4 per group).

FIG. 6H depicts changes in the ratio of tumor-infiltrating CD8⁺ T cells to Tregs in B16F10 tumors following a single intravenous dose of 5 mg/kg SP10784. Data is presented as mean±SEM (n=2-4 per group).

FIGS. 7A-FIG. 7C show IL-2 prodrug variants have extended pharmacokinetic (PK) profile compared to wild-type. Mean plasma concentration-time profile shows total antibody in C57BL/6 mice following IV bolus administration of a 0.8 mg/kg dose. Plasma concentrations were determined by ELISA using an anti-human IL-2 antibody. Data are presented as mean±standard deviation (SD).

FIG. 7A shows plasma concentrations of aldesleukin and IL-2 prodrug variants with increasing number of PEGs.

FIG. 7B shows plasma concentrations of aldesleukin and IL-2 prodrug variants with SC579 at different conjugation sites.

FIG. 7C shows plasma concentrations of aldesleukin and IL-2 prodrug variants with different PEG analogs.

FIG. 8 shows a sodium dodecyl-polyacrylamide gel electrophoresis (SDS-PAGE) of the kinetic release assay of various PEG aldesleukin variant S4-pAMF conjugates.

FIG. 9 shows the percentage of released aldesleukin variant SP9954 over time as quantified using densitometry analysis of the results shown in FIG. 8.

FIG. 10 shows the mean plasma concentration-time profile of IL-2 prodrug variants of non-releasable vs. releasable PEG analogs in C57BL/6 mice administered with 0.8 mg/kg IV bolus. Total antibody in plasma was determined by ELISA using an anti-human IL2 antibody. Data are presented as mean±standard deviation (SD).

FIG. 11A shows the mean±standard deviation of drug serum concentration-time profile after intravenous administration in cynomolgus monkeys on Day 0 and Day 7.

FIG. 11B shows the mean±standard deviation of sCD25 (B) serum concentration-time profile after intravenous administration in cynomolgus monkeys on Day 0 and Day 7.

FIG. 12A shows the mean±standard deviation of percent CD122 in NK cells as a surrogate for target engagement across treatment with either SP10784 or SP10477, administered IV on Day 0 and Day 7 (as indicated by the triangles) in cynomolgus monkeys at 0.1 or 0.3 mpk dose levels.

FIG. 12B shows the mean±standard deviation of percent % CD69 (B) in NK cells as a surrogate for target engagement across treatment with either SP10784 or SP10477, administered IV on Day 0 and Day 7 (as indicated by the triangles) in cynomolgus monkeys at 0.1 or 0.3 mpk dose levels.

FIG. 13A shows the mean±standard deviation body weight across SP10784 or SP10477, at 0.1 or 0.3 mpk dose levels, administered IV on Day 0 and Day 7 in cynomolgus monkeys.

FIG. 13B shows the mean±standard deviation eosinophils across SP10784 or SP10477, at 0.1 or 0.3 mpk dose levels, administered IV on Day 0 and Day 7 in cynomolgus monkeys.

FIG. 13C shows the mean±standard deviation lymphocytes (FIG. 13C) across SP10784 or SP10477, at 0.1 or 0.3 mpk dose levels, administered IV on Day 0 and Day 7 in cynomolgus monkeys.

FIG. 14A, FIG. 14B, and FIG. 14B together show the mean±standard deviation of peripheral lymphocyte populations represented as total cell counts (left panel) and percent Ki67 (proliferation marker; right panel) per 100 μL of blood across the indicated treatments with either SP10784 or SP10477, administered IV on Day 0 and Day 7 as indicated by the triangles in cynomolgus monkeys.

FIG. 15A shows individual data representing drug kinetics after SC or IV administration of SP10784 at 0.1 or 0.3 mpk dose levels in rhesus monkeys.

FIG. 15B shows individual data representing sCD25 serum concentration-time profile after SC or IV administration of SP10784 at 0.1 or 0.3 mpk dose levels in rhesus monkeys.

FIG. 16A shows the body weight from each individual animal after SC (from 0 day to 21 days) or IV (from 28 days to 49 days) administration of SP10784 at 0.1 or 0.3 mpk dose levels in rhesus monkeys.

FIG. 16B shows the eosinophils from each individual animal after SC (from 0 day to 21 days) or IV (from 28 days to 49 days) of SP10784 at 0.1 or 0.3 mpk dose levels in rhesus monkeys.

FIG. 16C shows the lymphocytes from each individual animal after SC (from 0 day to 21 days) or IV (from 28 days to 49 days) of SP10784 at 0.1 or 0.3 mpk dose levels in rhesus monkeys.

FIG. 17: Individual data representing the percent of CD122 in NK cells after SP10784 SC or IV administration in rhesus monkeys.

FIG. 18: Individual data representing the percent of CD69 in NK cells after SP10784 SC or IV administration in rhesus monkeys.

FIGS. 19A, 19B, and 19C show data from each individual animal represented as total cell counts (left panel) and percent Ki67 (proliferation marker; right panel) per 10E³ μL of blood across treatment with SP10784, administered SC or IV in rhesus monkeys.

### DETAILED DESCRIPTION OF THE INVENTION

### Definitions

As used herein, amino acid positions may be denoted as follows: Amino acid followed immediately by Position Number; e.g., Trp26 or W26. Where there is a substitution made, the substituted amino acid follows the Position Number; e.g., Trp26Cys or W26C. Trp26Cys or W26C in this non-limiting example denotes that the amino acid Tryptophan (Trp or W) at position 26 is changed to a Cysteine (Cys or C).

As used herein, the term “interleukin-2” or “IL-2” as used herein, refers to any wild-type or native IL-2 from any vertebrate source, including mammals such as primates (e.g. humans) and rodents (e.g., mice and rats), unless otherwise indicated. The term encompasses unprocessed IL-2 as well as any mature form of IL-2 that lacks the N-terminal leader signal sequence. The term also encompasses naturally occurring variants of IL-2, e.g. splice variants or allelic variants. The amino acid sequence of mature human IL-2 is shown in SEQ ID NO: 1. Unprocessed human IL-2 additionally comprises an N-terminal 20 amino acid signal peptide, which is absent in the mature human IL-2 molecule. Human mature IL-2 has three cysteine residues, namely, at positions C58, C105, and C125, of which C58 and C105 are linked intramolecularly by a disulfide bond (Tsuji et al., 1987, J. Biochem. 26:129-134); the cysteine positions are with respect to the amino acid positions in the amino acid sequence set forth in SEQ ID NO: 1. Recombinant mature human IL-2 with a deletion of the N-terminal alanine residue (desAla1 or desA1) and a substitution of serine for the cysteine at position 125 (C125S substitution) and expressed in E. coli has been found to be biologically active after in vitro refolding (Wang et al., 1984, Science, 224:1431-1433; Yun et al., 1988, Kor. J. Biochem. 22: 120-126). This molecule has the nonproprietary name of aldesleukin and has the amino acid sequence set forth in SEQ ID NO: 3. As used herein, the amino acid positions are with respect to the corresponding position in the amino acid sequence set forth in SEQ ID NO: 3 or SEQ ID NO: 2 (desA1-mature IL-2). Thus, the C125S substitution according to reference to the corresponding amino acid position in the amino acid sequence set forth in SEQ ID NO: 1 becomes a C124S substitution according to reference to the corresponding amino acid sequence in the amino acid sequence set forth in SEQ ID NO: 2. In the IL-2 variants, analogs, or prodrugs disclosed herein, reference to an amino acid position is according to the corresponding position of the amino acid in the amino acid sequence set forth in SEQ ID NO: 2.

As used herein, the term “IL-2 mutant” or “mutant IL-2 polypeptide” or “mutant IL-2” as used herein refers to an IL-2 polypeptide, either native human IL-2 or desAla1, C₁₂₅S IL-2 (e.g., aldesleukin) either of which has at least one amino acid substitution with a natural amino acid that affects or inhibits the interaction of IL-2 with CD25. IL-2 mutant polypeptides may further include a C125S or A substitution. The IL-2 mutant may be full-length, i.e., has an N-terminal alanine residue and truncated, i.e., lacks the N-terminal alanine. Unless otherwise indicated, an IL-2 mutant may be referred to herein as an IL-2 mutant peptide sequence, an IL-2 mutant polypeptide, IL-2 mutant protein or IL-2 mutant analog.

As used herein, the term “IL-2 moiety,” refers to a mutant IL-2 polypeptide, a mature IL-2 polypeptide, a C125S IL-2 polypeptide, and a desAla1, C125S IL-2 polypeptide (e.g., aldesleukin), any of which has human IL-2 activity and comprises at least one nonnatural amino acid having a functional group, e.g. an electrophilic group or a nucleophilic group, suitable for reaction with a reactive group comprising a nonpeptidic, water-soluble polymer. As an example, an IL-2 moiety may comprise one or more p-azidomethylphenylalanine (pAMF) residues; the azido functional group of the pAMF residue is suitable for conjugating in a triazole linkage to an alkyne reactive group linked to a nonpeptidic, water soluble polymer. As will be explained in further detail below, one of ordinary skill in the art can determine whether any given IL-2 moiety has IL-2 activity.

As used herein, the term “IL-2 prodrug” refers to an IL-2 conjugate comprising an IL-2 moiety comprising at least three nonnatural amino acids, each conjugated to a nonpeptidic, water-soluble polymer by a releasable linker in positions in the IL-2 polypeptide such that the IL-2 prodrug has undetectable or significantly attenuated binding to the IL-2α and IL-2 β receptors compared to the binding of the IL-2 moiety not conjugated to the nonpeptidic water-soluble polymer as determined by surface plasmon resonance and has the ability to bind the IL-2 receptors with a binding affinity (K_{D}) of about 6×10⁻⁷ M or less when the nonpeptidic, water-soluble polymer is released. The IL-2 moiety can be directly linked to the reactive group of, or within, the nonpeptidic, water-soluble polymer via a covalent bond, or the IL-2 moiety can be indirectly linked to the nonpeptidic, water-soluble polymer via the functional group of a linker linked to the nonpeptidic, water-soluble polymer. As an example, an IL-2 prodrug may comprise four p-azidomethylphenylalanine (pAMF) residues in which the azido functional group of each pAMF residue is conjugated in a triazole linkage to an alkyne reactive group linked to a nonpeptidic, water soluble polymer.

As used herein, the term “IL-2 drug” refers to an IL-2 conjugate comprising an IL-2 moiety comprising at least three nonnatural amino acids, each conjugated to a DBCO amine wherein the IL-2 drug has the ability to bind the IL-2α and IL-2 β receptors with a binding affinity (K_{D}) of about 6×10⁻⁷ M or less. As an example, an IL-2 prodrug may comprise four p-azidomethylphenylalanine (pAMF) residues in which the azido functional group of each pAMF residue is conjugated in a triazole linkage to an alkyne reactive group linked to a nonpeptidic, water soluble polymer.

As used herein, the term “significantly attenuated” refers to binding activity K_{D} as determined by surface plasmon resonance of 1×10⁻⁶ M or higher.

As used herein, the term “PEG,” “polyethylene glycol” and “poly(ethylene glycol)” as used herein, are interchangeable and encompass any nonpeptidic, water-soluble poly(ethylene oxide). Typically, PEGs for use in accordance with the invention comprise the following formula “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced, e.g., during a synthetic transformation. Throughout the specification and claims, it should be remembered that the term “PEG” includes structures having various terminal or “end capping” groups and so forth, e.g., monomethylpolyethylene glycol (mPEG) is PEG comprising a methyl capping group. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

As used herein, the term “water-soluble” as in a “nonpeptidic water-soluble polymer” polymer is any nonpeptidic polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

Molecular weight in the context of a nonpeptidic, water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques.

Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

As used herein, the term “active,” “reactive” or “activated” when used in conjunction with a particular functional group or reactive group, refers to a reactive functional or reactive group that reacts readily with an electrophile or a nucleophile on another molecule. This is in contrast to those groups that require strong catalysts or highly impractical reaction conditions in order to react (i.e., a “non-reactive” or “inert” group). As used herein, a reactive group interacts with a functional group to form a covalent linkage between the two.

As used herein, the term “linker” refers to a molecular moiety that is capable of forming at least two covalent bonds between a reactive group and a nonpeptidic, water-soluble polymer.

As used herein, the term “hydrolytically stable” linkage or bond refers to a chemical bond, typically a covalent bond, which is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, triazole, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

As used herein, the term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), glycine (Gly; G); histidine (His; H), isoleucine (Ile; I), leucine (Leu; L), lysine (Lys; K), methionine (Met; M), phenylalanine (Phe; F), praline (Pro; P), serine (Ser; S), threonine (Thr; T), tryptophan (Trp; W), tyrosine (Tyr; Y), and valine (Val; V).

Naturally encoded amino acids are the proteinogenic amino acids known to those of skill in the art. They include the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids.

As used herein, the term “nonnatural amino acid” or “NNAA” or “unnatural amino acid” or “UAA” or “UNAA” all refer to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof. Examples of nonnatural amino acids include but are not limited to p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine.

As used herein, the term “orthogonal” refers to a molecule (e.g., an orthogonal tRNA (O-tRNA) and/or an orthogonal aminoacyl tRNA synthetase (O-RS)) that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficient, less than 10% efficient, less than 5% efficient, or less than 1% efficient, of an orthogonal tRNA to function with an endogenous tRNA synthetase compared to an endogenous tRNA to function with the endogenous tRNA synthetase, or of an orthogonal aminoacyl-tRNA synthetase to function with an endogenous tRNA compared to an endogenous tRNA synthetase to function with the endogenous tRNA. The orthogonal molecule lacks a functional endogenous complementary molecule in the cell. For example, an orthogonal tRNA in a cell is aminoacylated by any endogenous RS of the cell with reduced or even zero efficiency, when compared to aminoacylation of an endogenous tRNA by the endogenous RS. In another example, an orthogonal RS aminoacylates any endogenous tRNA in a cell of interest with reduced or even zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., 50% efficiency, 60% efficiency, 70% efficiency, 75% efficiency, 80% efficiency, 90% efficiency, 95% efficiency, or 99% or more efficiency) to that of a corresponding tRNA/RS endogenous pair.

As used herein, the term “complementary” refers to components of an orthogonal pair, O-tRNA and O-RS that can function together, e.g., the O-RS aminoacylates the O-tRNA.

As used herein, the term “translation system” refers to the collective set of components that incorporate a naturally occurring amino acid into a growing polypeptide chain (protein). Components of a translation system can include, e.g., ribosomes, tRNAs, synthetases, mRNA, amino acids, and the like. The components for an orthogonal translation system include for example O-RS, O-tRNAs, nonnatural amino acids, etc., which can be added to an in vitro or in vivo translation system, e.g., cell-free, a eukaryotic cell, e.g., a yeast cell, a mammalian cell, a plant cell, an algae cell, a fungus cell, an insect cell, and/or the like.

As used herein, “combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

As used herein, the term “medical delivery device” or “device” or “medication delivery device” are used interchangeably and has the definition set forth in Section 201(h) of the Food, Drug, and Cosmetic Act, which defines a device as an instrument, apparatus, implement, machine, contrivance, implant, in vitro reagent, or other similar or related article, including a component part, or accessory which is: (i) recognized in the official National Formulary, or the United States Pharmacopoeia, or any supplement to them, (ii) intended for use in the diagnosis of disease or other conditions, or in the cure, mitigation, treatment, or prevention of disease, in man or other animals, or (iii) intended to affect the structure or any function of the body of man or other animals, and which does not achieve its primary intended purposes through chemical action within or on the body of man or other animals and which does not achieve its primary intended purposes through chemical action within or on the body of man or other animals and which is not dependent upon being metabolized for the achievement of its primary intended purposes. The term “device” does not include software functions excluded pursuant to section 520(o). Examples of devices include medical pens and autoinjectors.

As used herein, the term “treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of a protein or composition that when administered to a subject is effective to treat a disease or disorder. In some embodiments, a therapeutically effective amount or effective amount refers to an amount of a protein or composition that when administered to a subject is effective to prevent or ameliorate a disease, the progression of the disease, or result in amelioration of symptoms.

As used herein, the term “substantially similar” or “substantially the same,” as used herein, denotes a sufficiently high degree of similarity between two or more numeric values, for example, receptor binding affinity, EC₅₀, etc., such that one of skill in the art would consider the difference between the two or more values to be of little or no biological and/or statistical significance within the context of the biological characteristic measured by said value. In some embodiments, the two or more substantially similar values may be within 5% to 100% of each other.

As used herein, the term “Alkoxy” and “alkoxyl,” refer to the group-OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

As used herein, the term “alkoxyamine,” as used herein, refers to the group-alkylene-O—NH2, wherein alkylene is as defined herein. In some embodiments, alkoxyamine groups can react with aldehydes to form oxime residues. Examples of alkoxyamine groups include —CH2CH2—O—NH2 and —CH2—O—NH2.

As used herein, the term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆alkyl, and the like. In certain embodiments, the alkyl group is selected from the group consisting of methyl, ethyl, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with one, two, or three groups independently selected from a halogen (e.g., fluoro (F), chloro (Cl), bromo (Br), or iodo (I)), alkyl, haloalkyl, hydroxyl, amino, alkylamino, and alkoxy. In some embodiments, alkyl is unsubstituted.

As used herein, the term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

As used herein, the term “Alkenyl” refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

As used herein, the term “Alkenylene” refers to a divalent alkenyl as defined herein. Lower alkenylene is, for example, C₂-C₆-alkenylene.

As used herein, the term “Alkynyl” refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

As used herein, the term “Alkynylene” refers to a divalent alkynyl as defined herein. Lower alkynylene is, for example, C₂-C₆-alkynylene.

As used herein, the term “Amino” refers to —NH₂.

As used herein, the term “alkylamino,” as used herein, and unless otherwise specified, refers to the group —NHR″ where R″ is, for example, C₁₋₁₀alkyl, as defined herein. In certain embodiments, alkylamino is C₁₋₆alkylamino.

As used herein, the term “dialkylamino,” as used herein, and unless otherwise specified, refers to the group —NR″R″ where each R″ is independently C₁₋₁₀alkyl, as defined herein. In certain embodiments, dialkylamino is di-C₁₋₆alkylamino.

As used herein, the term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (e.g., Greene, et al., Protective Groups in Organic Synthesis, John Wiley and Sons, Second Edition, 1991); and wherein the aryl in the arylamino and aryloxy substituents are not further substituted.

As used herein, the term “arylamino,” as used herein, and unless otherwise specified, refers to an —NR′R″ group where R′ is hydrogen or C₁-C₆-alkyl; and R″ is aryl, as defined herein.

As used herein, the term “arylene,” as used herein, and unless otherwise specified, refers to a divalent aryl group, as defined herein.

As used herein, the term “aryloxy,” as used herein, and unless otherwise specified, refers to an

—OR group where R is aryl, as defined herein.

As used herein, the term “Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

As used herein, the term “Aralkylene” refers to an —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

As used herein, the term “Carboxyl” or “carboxy” refers to —C(O)OH or —COOH.

As used herein, the term “cycloalkyl,” as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms (i.e., C₃ to C₁₀ cycloalkyl). In some embodiments, the cycloalkyl has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

As used herein, the term “cycloalkylene,” as used herein refers to a divalent cycloalkyl group, as defined herein. In certain embodiments, the cycloalkylene group is cyclopropylene

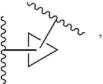

cyclobutylene

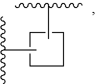

cyclopentylene

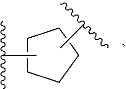

cyclohexylene

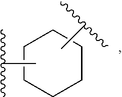

cycloheptylene

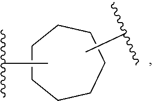

and the like. Lower cycloalkylene refers to a C₃-C₆-cycloalkylene.

As used herein, the term “cycloalkylalkyl,” as used herein, unless otherwise specified, refers to an alkyl group, as defined herein, substituted with one or two cycloalkyl, as defined herein.

As used herein, the term “ester,” as used herein, refers to —C(O)OR or —COOR where R is alkyl, as defined herein.

As used herein, the term “fluorene” as used herein refers to

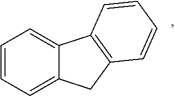 [SMILES: c1ccc2c(c1)Cc1ccccc1-2]

wherein any one or more carbons bearing one or more hydrogens can be substituted with a chemical functional group as described herein.

As used herein, the term “haloalkyl” refers to an alkyl group, as defined herein, substituted with one or more halogen atoms (e.g., in some embodiments one, two, three, four, or five) which are independently selected.

As used herein, the term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

As used herein, the term “heteroalkylene,” as used herein, refers to a divalent heteroalkyl, as defined herein. “Substituted heteroalkylene” refers to a divalent heteroalkyl, as defined herein, substituted as described for heteroalkyl.

As used herein, the term “heterocycloalkyl” refers to a monovalent, monocyclic, or multicyclic non-aromatic ring system, wherein one or more of the ring atoms are heteroatoms independently selected from oxygen (O), sulfur (S), and nitrogen (N) (e.g., where the nitrogen or sulfur atoms may be optionally oxidized, and the nitrogen atoms may be optionally quaternized) and the remaining ring atoms of the non-aromatic ring are carbon atoms. In certain embodiments, heterocycloalkyl is a monovalent, monocyclic, or multicyclic fully-saturated ring system. In certain embodiments, the heterocycloalkyl group has from three to twenty, from three to fifteen, from three to ten, from three to eight, from four to seven, from four to eleven, or from five to six ring atoms. The heterocycloalkyl may be attached to a core structure at any heteroatom or carbon atom which results in the creation of a stable compound. In certain embodiments, the heterocycloalkyl is a monocyclic, bicyclic, tricyclic, or tetracyclic ring system, which may include a fused or bridged ring system and in which the nitrogen or sulfur atoms may be optionally oxidized, and/or the nitrogen atoms may be optionally quaternized. In some embodiments, heterocycloalkyl radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocycloalkyl may also be optionally substituted as described herein. In certain embodiments, heterocycloalkyl is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heterocycloalkyl group may comprise one, two, three, or four heteroatoms. Those of skill in the art will recognize that a 4-membered heterocycloalkyl may generally comprise one or two heteroatoms, a 5 or 6-membered heterocycloalkyl may generally comprise one, two, or three heteroatoms, and a 7- to 10-membered heterocycloalkyl may generally comprise one, two, three, or four heteroatoms.

As used herein, the term “Heterocycloalkylene” refers to a divalent heterocycloalkyl as defined herein.

As used herein, the term “heteroaryl” refers to a monovalent monocyclic aromatic group and/or multicyclic aromatic group, wherein at least one aromatic ring contains one or more heteroatoms independently selected from oxygen, sulfur, and nitrogen in the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

As used herein, the term “heteroarylene” refers to a divalent heteroaryl group, as defined herein. “Substituted heteroarylene” is a heteroarylene substituted as defined for aryl.

As used herein, the term “Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, muconic acid, and the like; or (2) salts formed when an acidic proton present in the parent compound either (a) is replaced by a metal ion, for example, an alkali metal ion, an alkaline earth ion, or an aluminum ion, or alkali metal or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

As used herein, the term “pAMF,” “pAMF residue,” or “pAMF mutation” refers to a variant phenylalanine residue (i.e., para-azidomethyl-L-phenylalanine) added or substituted into a polypeptide.

As used herein, the terms “subject” and “patient” are used interchangeably. The terms “subject” and “subjects” refer to an animal, such as a mammal including a non-primate (e.g, a cow, pig, horse, cat, dog, rat, and mouse) and a primate (e.g., a monkey, such as a cynomolgous monkey, a chimpanzee, and a human), and in certain embodiments, a human. In certain embodiments, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound or conjugate provided herein. In certain embodiments, a therapeutic agent is an agent which is known to be useful for, or has been or is currently being used for the treatment or prevention of a disorder or one or more symptoms thereof.

As used herein, the term “Therapeutically effective amount” refers to an amount of a compound or composition that, when administered to a subject for treating a condition, is sufficient to effect such treatment for the condition. A “therapeutically effective amount” can vary depending on, inter alia, the compound, the disease or disorder and its severity, and the age, weight, etc., of the subject to be treated.

In some chemical structures illustrated herein, certain substituents, chemical groups, and atoms are depicted with a curvy/wavy/wiggly line

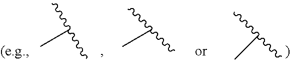

that intersects a bond or bonds to indicate the atom through which the substituents, chemical groups, and atoms are bonded. For example, in some structures, such as but not limited to,

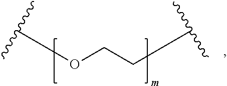

this curvy/wavy/wiggly line indicates the atoms in the backbone of a conjugate, linker-fluorenylmethoxycarbonyl compound structure to which the illustrated chemical entity is bonded. In some structures, such as but not limited to

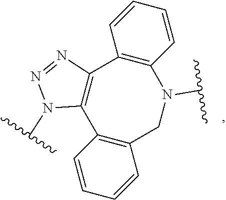 [SMILES: *N1Cc2ccccc2-c2c(nnn2*)-c2ccccc21]

this curvy/wavy/wiggly line indicates the atoms in the macromolecule as well as the atoms in the backbone of a conjugate, linker-fluorenylmethoxycarbonyl compound structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

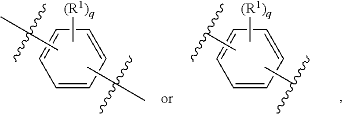

wherein subscript q is an integer from zero to four and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

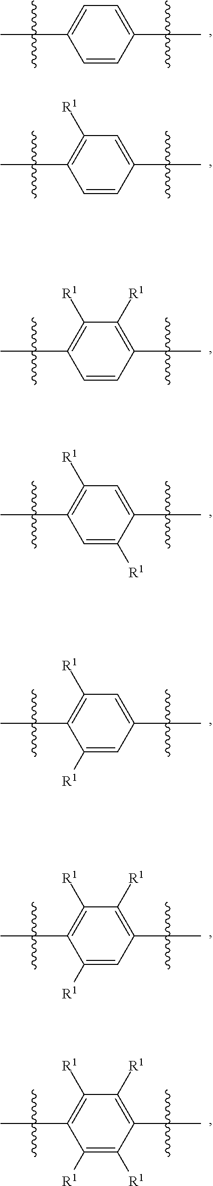

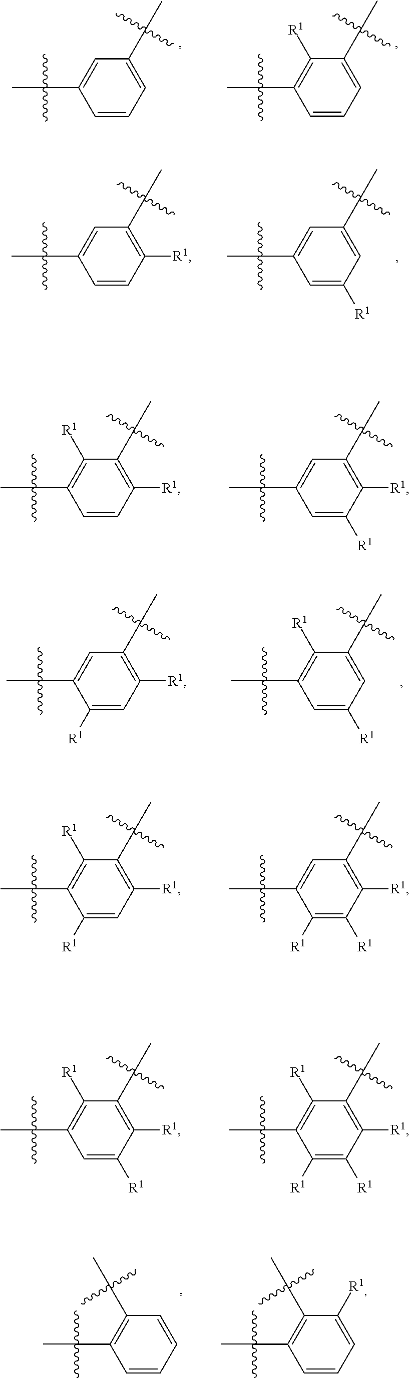

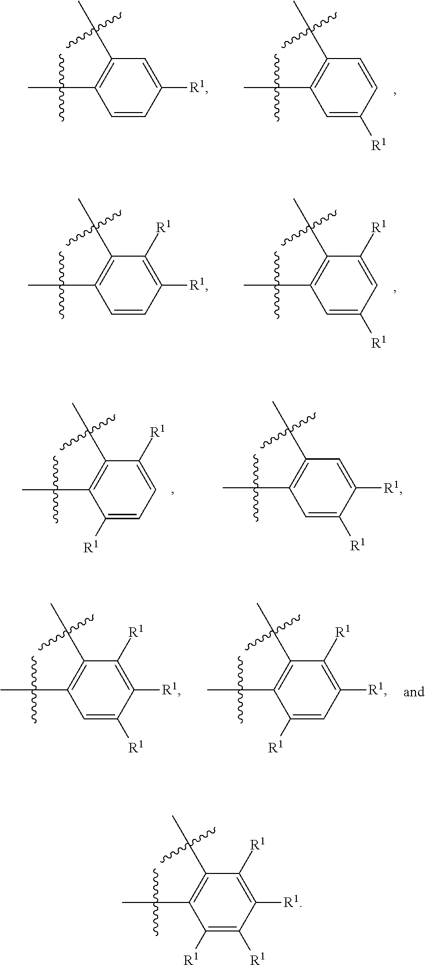

As used herein, the term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### IL-2 Prodrugs

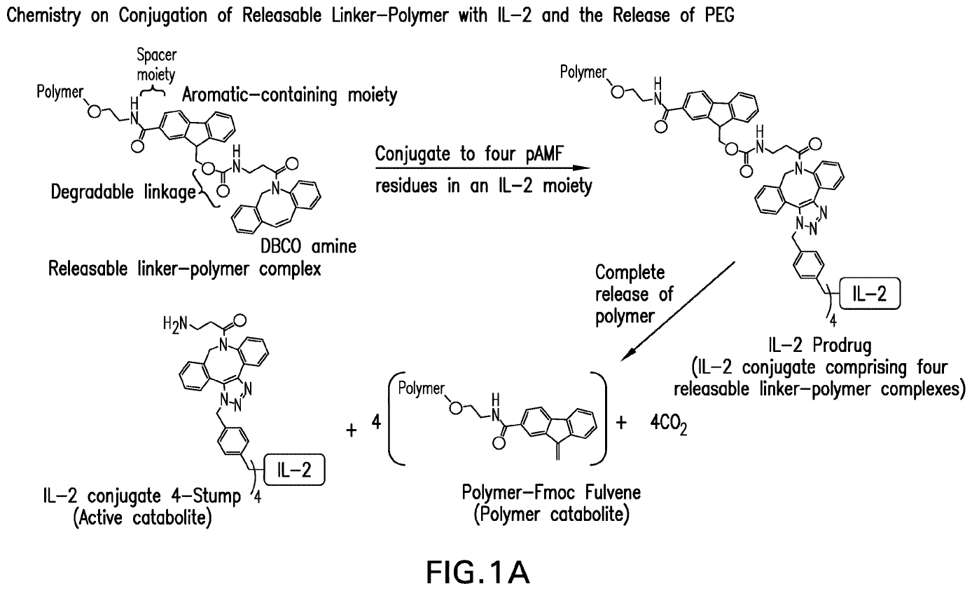

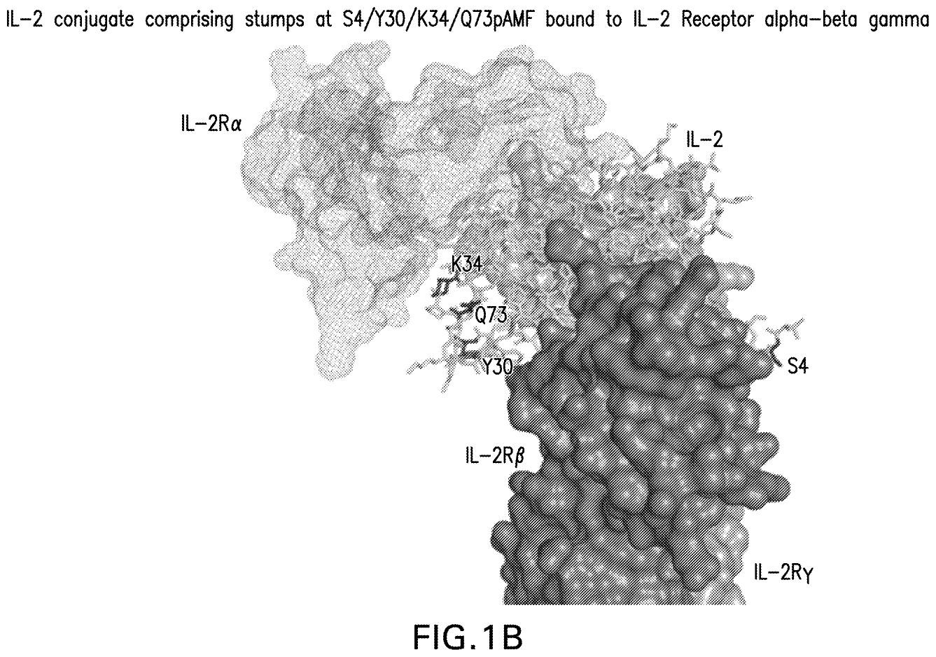

The present invention provides IL-2 prodrugs comprising an IL-2 conjugate comprising at least three (and in specific embodiments, three or four) nonnatural amino acids (NNAA) conjugated to a nonpeptidic, water-soluble polymer by a releasable linker comprising a degradable linkage therein (releasable linker-polymer complex). The NNAAs are located in positions within the IL-2 polypeptide such that the IL-2 conjugate comprising the releasable linker-polymer complex displays undetectable or significantly attenuated binding to both the IL-2a receptor (IL-2Rα) and IL-2β (IL-2Rβ) receptor as determined by surface plasmon resonance compared to binding of an IL-2 moiety comprising the same NNAA substitutions and displays no detectable IL-2 activity at the IL-2Rαβγ receptor complex or IL-2Rβγ receptor complex. Degradation of the degradable linkage over time produces a second IL-2 conjugate that lacks the nonpeptidic, water soluble polymer but which contains that portion of the releasable linker-polymer complex residing between the degradable linkage and the NNAA. FIG. 1A illustrates the conjugation of an exemplary releasable linker-polymer complex comprising a degradable linkage to an IL-2 moiety comprising the NNAA para-azidomethylphenylalanine (pAMF) at positions S4, Y30, K34, and Q73 (with reference to the amino acid positions shown in SEQ ID NO: 2 or 3) to provide the exemplary IL-2 prodrug. Release of the releasable linker-polymer complex from the prodrug following degradation of the degradable linkage produces a second IL-2 conjugate that comprises a “stump” comprising a remnant of the releasable linker-polymer complex between the degradable linkage and the NNAA.

As shown in the Examples, the inventors discovered an embodiment wherein conjugating the releasable linker-polymer complex comprising a degradable linkage to three or four amino acids selected from positions S4, Y30, K34, Q73, and V114 (positions are in reference to the amino acid positions shown in SEQ ID NO: 2 or 3), produces an IL-2 conjugate that displays undetectable or significantly attenuated binding to both IL-2Rα and the IL-2Rβ. In a particular embodiment, the IL-2 conjugate comprises the releasable linker-polymer complex comprising a degradable linkage conjugated to four NNAA selected from positions S4, Y30, K34, Q73, and V114. In a further embodiment, the IL-2 conjugate comprises the releasable linker-polymer complex comprising a degradable linkage conjugated to four NNAA located in positions S4, Y30, K34, and Q73. In a further embodiment, the IL-2 conjugate comprises the releasable linker-polymer complex comprising a degradable linkage conjugated to four NNAA located in positions S4, Y30, K34, and V114. FIG. 1B shows the positions S4, Y30, K34, and Q73 in relationship to the binding of the second IL-2 conjugate to the IL-2Rα and the IL-2Rβ. Table 13 shows that stumps in positions S4, Y30, K34, and Q73 of the second IL-2 conjugate do not significantly impact binding of the second IL-2 conjugate to the IL-2Rα and the IL-2Rβ.

It was uncertain during development as to what the effect of the stump at these positions might have on binding of the second IL-2 conjugate to IL-2Rα and the IL-2Rβ or on the activity of the IL-2Rαβγ receptor complex or IL-2Rβγ receptor complex, particularly as it is known in the art that amino acid substitutions at various positions in the IL-2 polypeptide can significantly reduce binding to the IL-2α receptor.

IL-2 amino acid positions that are believed to be involved in binding to the IL-2Rα and the IL-2Rβ have been disclosed for example in the following. U.S. Pat. No. 9,732,134, which discloses that IL-2 residues believed to contact IL-2Rα include K34, R37, F41, K42, F43, Y44, E60, E61, K63, P64, E67, V68, L71, and Y106; IL-2Rβ include L11, Q12, H15, L18, D19, M22, R80, D83, S86, N87, V90, 191, and E94; and IL-2Rγ include Q10, L17, Q21, E109, N118, T122, Q125, S126, 1128, S129, and T132 (positions are in reference to the amino acid sequence set forth in SEQ ID NO: 2 herein).

U.S. Pat. No. 11,077,195, which discloses that at least one amino acid at position K34, R37, T40, F41, F43, K42, Y44, E61, P64, E67, V68, or L71 of the IL-2 polypeptide as referenced by the amino acid positions set forth in SEQ ID NO:2 herein may be replaced with a nonnatural amino acid conjugated to a polymer to provide an IL-2 conjugate with reduced binding to the IL-2Rα while retaining significant binding to the IL-2Rβγ signaling complex to form an IL-2/IL-2Rβγ complex.

WO2019028425 and WO2021050554, which disclose that at least one amino acid at position P1, T2, S3, S4, S5, T6, K7, K8, Q10, L11, E14, H15, L17, L18, D19, Q21, M22, N25, G26, N28, N29, Y30, K31, K34, T36, M45, K46, K47, A49, T50, E51, K52, H54, Q56, E59, E66, N70, Q73, S74, K75, N76, F77, H78, R80, P81, R82, D83, S86, N87, N88, V90, 191, L93, E94, K96, G97, S98, E99, T100, T101, F102, M103, C104, E105, Y106, A107, D108, E109, T110, A111, T112, E115, N118, R119, T122, A124, Q125, S126, S129, T130, L131, and T132 of the IL-2 polypeptide as referenced by the amino acid positions set forth in SEQ ID NO:2 herein may be replaced with a nonnatural amino acid conjugated to a polymer to provide an IL-2 conjugate with reduced receptor signaling potency to the IL-2Rβγ or recruitment of the IL-2Rγ subunit to the IL-2/IL-2Rβ complex while retaining significant activation of the IL-2Rαβγ signaling complex.

In contrast to the above IL-2 conjugates, the applicant has discovered that the IL-2 conjugates disclosed herein display undetectable or significantly attenuated binding to both IL-2Rα and IL-2Rβ as determined by surface plasmon resonance compared to binding of an IL-2 moiety comprising the same NNAA substitutions, and thus displaying undetectable or significantly attenuated binding activity at the IL-2Rαβγ or IL-2Rβγ signaling complex and that the second conjugate formed following release of the releasable linker-polymer complex can bind the IL-2Rα and IL-2Rβ and there is detectable activity at the IL-2Rαβγ or IL-2Rβγ signaling complex. Thus, the second IL-2 conjugate stumps formed following release of the releasable linker-polymer complex from the IL-2 conjugate do not significantly interfere with binding to IL-2Rα and IL-2Rβ and the activity of the IL-2Rαβγ and IL-2Rβγ signaling complexes.

In an embodiment of the present invention, the IL-2 conjugate comprises an IL-2 polypeptide comprising an amino acid sequence having at least 80% identity to the amino acid sequence set forth in SEQ ID NO: 2 or SEQ ID NO: 3 with the proviso that the IL-2 polypeptide comprises (a) at least three NNAAs conjugated to nonpeptidic, water-soluble polymers at positions selected from positions S4, Y30, K34, Q73, and V114 or (b) four NNAAs conjugated to nonpeptidic, water-soluble polymers at positions selected from positions S4, Y30, K34, Q73, and V114 in reference to the amino acid positions within SEQ ID NO: 2 and any other amino acid substitutions whether by a natural amino acid or a nonnatural amino acid are in positions in the IL-2 polypeptide that do not reduce or abrogate binding to the IL-2Rα and the IL-2Rβ.

In a further embodiment, the IL-2 conjugate comprises an IL-2 polypeptide comprising an amino acid sequence having at least 80% identity to the amino acid sequence set forth in SEQ ID NO: 2 or SEQ ID NO: 3 with the proviso that the IL-2 polypeptide comprises (a) four NNAAs conjugated to nonpeptidic, water-soluble polymers at positions S4, Y30, K34, and Q73 in reference to the amino acid positions within SEQ ID NO: 2 or (b) four NNAAs conjugated to nonpeptidic, water-soluble polymers at positions S4, Y30, K34, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In an embodiment of the present invention, the IL-2 conjugate comprises an IL-2 polypeptide comprising an amino acid sequence having at least 85% identity to the amino acid sequence set forth in SEQ ID NO: 2 or SEQ ID NO: 3 with the proviso that the IL-2 polypeptide comprises (a) at least three NNAAs conjugated to nonpeptidic, water-soluble polymers at positions selected from positions S4, Y30, K34, Q73, and V114 in reference to the amino acid positions within SEQ ID NO: 2 or (b) four NNAAs conjugated to nonpeptidic, water-soluble polymers at positions selected from positions S4, Y30, K34, Q73, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In a further embodiment, IL-2 conjugate comprises an IL-2 polypeptide comprising an amino acid sequence having at least 85% identity to the amino acid sequence set forth in SEQ ID NO: 2 or SEQ ID NO: 3 with the proviso that the IL-2 polypeptide comprises (a) four NNAAs conjugated to nonpeptidic, water-soluble polymers at positions S4, Y30, K34, and Q73 in reference to the amino acid positions within SEQ ID NO: 2 or (b) four NNAAs conjugated to nonpeptidic, water-soluble polymers at positions S4, Y30, K34, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In an embodiment of the present invention, the IL-2 conjugate comprises an IL-2 polypeptide comprising an amino acid sequence having at least 90% identity to the amino acid sequence set forth in SEQ ID NO: 2 or SEQ ID NO: 3 with the proviso that the IL-2 polypeptide comprises (a) at least three NNAAs conjugated to nonpeptidic, water-soluble polymers at positions selected from positions S4, Y30, K34, Q73, and V114 in reference to the amino acid positions within SEQ ID NO: 2 or (b) four NNAAs conjugated to nonpeptidic, water-soluble polymers at positions selected from positions S4, Y30, K34, Q73, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In a further embodiment, IL-2 conjugate comprises an IL-2 polypeptide comprising an amino acid sequence having at least 90% identity to the amino acid sequence set forth in SEQ ID NO: 2 or SEQ ID NO: 3 with the proviso that the IL-2 polypeptide comprises (a) four NNAAs conjugated to nonpeptidic, water-soluble polymers at positions S4, Y30, K34, and Q73 in reference to the amino acid positions within SEQ ID NO: 2 or (b) four NNAAs conjugated to nonpeptidic, water-soluble polymers at positions S4, Y30, K34, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In an embodiment of the present invention, the IL-2 conjugate comprises an IL-2 polypeptide comprising an amino acid sequence having at least 95% identity to the amino acid sequence set forth in SEQ ID NO: 2 or SEQ ID NO: 3 with the proviso that the IL-2 polypeptide comprises (a) at least three NNAAs conjugated to nonpeptidic, water-soluble polymers at positions selected from positions S4, Y30, K34, Q73, and V114 in reference to the amino acid positions within SEQ ID NO: 2 or (b) four NNAAs conjugated to nonpeptidic, water-soluble polymers at positions selected from positions S4, Y30, K34, Q73, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2Rα and the IL-2Rβ.

In a further embodiment, IL-2 conjugate comprises an IL-2 polypeptide comprising an amino acid sequence having at least 95% identity to the amino acid sequence set forth in SEQ ID NO: 2 or SEQ ID NO: 3 with the proviso that the IL-2 polypeptide comprises (a) four NNAAs conjugated to nonpeptidic, water-soluble polymers at positions S4, Y30, K34, and Q73 in reference to the amino acid positions within SEQ ID NO: 2 or (b) four NNAAs conjugated to nonpeptidic, water-soluble polymers at positions S4, Y30, K34, and V114 in reference to the amino acid positions within SEQ ID NO: 2; and any other amino acid substitutions whether by a natural amino acid or a nonnatural amino acid are in positions in the IL-2 polypeptide that do not reduce or abrogate binding to the IL-2α receptor and the IL-2β receptor.

In an embodiment of the present invention, the IL-2 conjugate comprises an IL-2 polypeptide comprising an amino acid sequence having at least 100% identity to the amino acid sequence set forth in SEQ ID NO: 2 or SEQ ID NO: 3 with the proviso that the IL-2 polypeptide comprises (a) at least three NNAAs conjugated to nonpeptidic, water-soluble polymers at positions selected from positions S4, Y30, K34, Q73, and V114 in reference to the amino acid positions within SEQ ID NO: 2 or (b) four NNAAs conjugated to nonpeptidic, water-soluble polymers at positions selected from positions S4, Y30, K34, Q73, and V114 in reference to the amino acid positions within SEQ ID NO: 2.

In a further embodiment, IL-2 conjugate comprises an IL-2 polypeptide comprising an amino acid sequence having at least 100% identity to the amino acid sequence set forth in SEQ ID NO: 2 or SEQ ID NO: 3 with the proviso that the IL-2 polypeptide comprises (a) four NNAAs conjugated to nonpeptidic, water-soluble polymers at positions S4, Y30, K34, and Q73 in reference to the amino acid positions within SEQ ID NO: 2 or (b) four NNAAs conjugated to nonpeptidic, water-soluble polymers at positions S4, Y30, K34, and V114 in reference to the amino acid positions within SEQ ID NO: 2.

While the IL-2 conjugates of the present invention do not comprise amino acid substitutions that reduce or abrogate second IL-2 conjugate binding to the IL-2Rα and the IL-2Rβ or the IL-2Rαβγ or IL-2Rβγ signaling complex, they may include amino acid substitutions that enhance binding of the second IL-2 conjugate to the IL-2α receptor and the IL-2 receptor or enhance activity at the IL-2Rαβγ or IL-2Rβγ signaling complex. In further embodiments, the IL-2 conjugates further do not comprise amino acid substitutions disclosed above that reduce or abrogate binding to the IL-2γ receptor when the releasable linker-polymer complex is released from the IL-2 conjugate but may include amino acid substitutions that enhance binding of the second IL-2 conjugate to the IL-2γ receptor.

In particular embodiments, the IL-2 moiety may comprise the amino acid sequence set forth in SEQ ID No: 4, 5, 6, 7, 8, or 9. In particular embodiments, the IL-2 conjugate may comprise the amino acid sequence set forth in SEQ ID NO: 10, 11, 12, 13, 14, 15, 16, 17, or 18. In particular embodiments, the above IL-2 conjugates may further comprise an N-terminal methionine residue or an N-terminal methionine-alanine dipeptide.

### Releasable Linker

The releasable linker may be represented by the following formula
[Reactive Group]-[Degradable Linkage]-[Aromatic-Containing Moiety].

The releasable linker comprises a distal end and a proximal end. The proximal end comprises a reactive group, which facilitates conjugation of the releasable linker to the functional group of an NNAA within the IL-2 moiety to produce an IL-2 conjugate. The distal end comprises an aromatic-containing moiety comprising an attachment site for a nonpeptidic, water-soluble polymer. Between the reactive group and the aromatic-containing moiety is positioned a degradable linkage that is susceptible to degradation over time. The rate of degradation of the degradable linkage may be controlled by the position of the attachment site for the nonpeptidic, water-soluble polymer, the positioning of none or one or more optional electron-withdrawing moieties within the aromatic-containing moiety, and/or a spacer moiety that covalently links the nonpeptidic, water-soluble polymer to the aromatic-containing moiety.

### Reactive Group

Reactive groups can react via any suitable reaction mechanism known to those of skill in the art. In certain embodiments, a reactive group reacts with a functional group through a [3+2] alkyne-azide cycloaddition reaction, inverse-electron demand Diels-Alder ligation reaction, thiol-electrophile reaction, or carbonyl-oxyamine reaction, as described in detail herein. In certain embodiments, the reactive group comprises an alkyne, strained alkyne, tetrazine, thiol, para-acetyl-phenylalanine residue, oxyamine, maleimide, or azide.

In certain embodiments, the reactive group is a dibenzocyclooctyne (DBCO) group having the formula

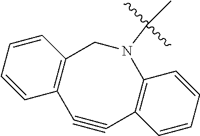 [SMILES: *N1Cc2ccccc2C#Cc2ccccc21]

wherein the wavy line indicates a bond to the remainder of the releasable linker.

In particular embodiments, the DBCO group further comprises an amine to provide a reactive group comprising a DBCO amine having the formula

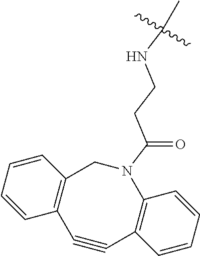 [SMILES: *NCCC(=O)N1Cc2ccccc2C#Cc2ccccc21]

wherein the wavy line indicates a bond to the remainder of the releasable linker.

DBCO groups and derivatives thereof have been described for example in U.S. Pat. No. 8,541,625; RE47539; U.S. Patent Pub. No. 2016010107999; EP3004062, and WO2006050262, each of which is incorporated herein in its entirety. Additional functional groups are described in, for example, U.S. Patent Publication No. 2014/0356385, U.S. Patent Publication No. 2013/0189287, U.S. Patent Publication No. 2013/0251783, U.S. Pat. Nos. 8,703,936, 9,145,361, 9,222,940, and 8,431,558.

After conjugation, a divalent residue of the reactive group is formed and is bonded to the NNAA functional group, which may be represented by the formula [NNAA]-[Divalent residue]-[Degradable Linkage]-[Aromatic-containing moiety]. The structure of the divalent residue is determined by the type of conjugation reaction employed to form the conjugate. For example, in certain embodiments, when a conjugate is formed through a [3+2] alkyne-azide cycloaddition reaction, the divalent residue of the reactive group comprises a 1,2,3-triazole ring or fused cyclic group comprising a 1,2,3-triazole ring.

In certain embodiments when a conjugate is formed through a strain-promoted [3+2] alkyne-azide cycloaddition (SPAAC) reaction between a DBCO group and an azido group of a NNAA, the divalent residue of the functional group comprises the 1,2,3-triazole as shown by Formula I:

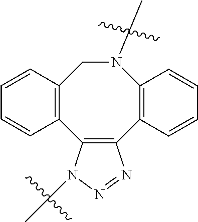 [SMILES: *N1Cc2ccccc2-c2c(nnn2*)-c2ccccc21]

or regioisomer

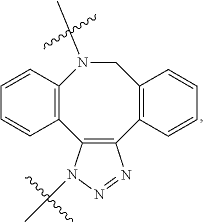 [SMILES: *N1Cc2ccccc2-c2nnn(*)c2-c2ccccc21]

wherein the bottom wavy line indicates a bond of the NNAA and the top wavy line indicates a bond to the remainder of the releasable linker.

In certain embodiments when a conjugate is formed through a strain-promoted [3+2] alkyne-azide cycloaddition (SPAAC) reaction between a DBCO group and an azido group of a NNAA, the divalent residue of the functional group comprises the 1,2,3-triazole as shown by Formula II:

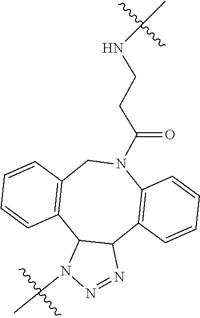 [SMILES: *NCCC(=O)N1Cc2ccccc2C2C(N=NN2*)c2ccccc21]

or regioisomer

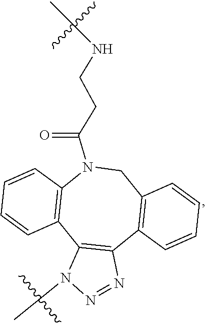 [SMILES: *NCCC(=O)N1Cc2ccccc2-c2nnn(*)c2-c2ccccc21]

wherein the bottom wavy line indicates a bond of the NNAA and the top wavy line indicates a bond to the remainder of the releasable linker.

### Aromatic-Containing Moiety

The aromatic-containing moiety is located at the distal end of the releasable linker as represented by the formula
[Reactive Group]-[Degradable Linkage]-[Aromatic-containing moiety].

The aromatic-containing moiety provides an attachment site for one or more nonpeptidic, water soluble polymers and having an ionizable hydrogen atom. Although most any aromatic-containing moiety having an ionizable hydrogen atom can be used, the aromatic-containing moiety must provide a site or sites for attachment of various components. In addition, it must be recognized that the aromatic-containing moiety does not itself have to be completely aromatic. The aromatic-containing moiety may, for example, contain one or more separate aromatic moieties optionally linked to each other directly or indirectly through a spacer moiety comprising one or more atoms. Releasable linkers comprising aromatic-containing moieties has been disclosed in WO2006138572, which is incorporated herein by reference in its entirety.

In an exemplary embodiment, the aromatic-containing moiety comprises Formula III:

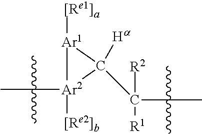

wherein Ar¹ is a first aromatic moiety; Ar² is a second aromatic moiety; Hα is an ionizable hydrogen atom; R¹ is H or an organic radical; R² is H or an organic radical; R^{e1}, when present, is a first electron altering group; R^{e2}, when present, is a second electron altering group; (a) is either zero or one; (b) is either zero or one; the wavy line on the left indicates a bond to a polymer or a spacer moiety linked to a polymer and the wavy line on the right indicates a bond to a degradable linkage. In particular embodiments, (a) is zero and (b) is zero; (a) is one and (b) is zero; or (a) is zero and (b) is one.

In a further embodiment, the aromatic-containing moiety comprises Formula IV

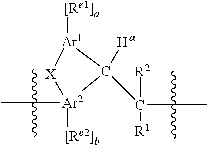

wherein Ar¹ is a first aromatic moiety; Ar² is a second aromatic moiety; Hα is an ionizable hydrogen atom; R¹ is H or an organic radical; R² is H or an organic radical; X is spacer moiety; R^{e1}, when present, is a first electron altering group; R^{e2}, when present, is a second electron altering group; (a) is either zero or one; (b) is either zero or one; the wavy line on the left indicates a bond to a polymer or a spacer moiety linked to a polymer and the wavy line on the right indicates a bond to a degradable linkage. In particular embodiments, (a) is zero and (b) is zero; (a) is one and (b) is zero; or (a) is zero and (b) is one.

In a further embodiment, the aromatic-containing moiety comprises Formula V

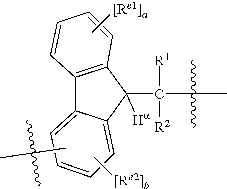

wherein Hα is an ionizable hydrogen atom; R¹ is H or an organic radical; R² is H or an organic radical; R^{e1}, when present, is a first electron altering group; R^{e2}, when present, is a second electron altering group; (a) is either zero or one; (b) is either zero or one; the wavy line on the left indicates a bond to a polymer or a spacer moiety linked to a polymer and the wavy line on the right indicates a bond to a degradable linkage. In particular embodiments, (a) is zero and (b) is zero; (a) is one and (b) is zero; or (a) is zero and (b) is one.

In a further embodiment, the aromatic-containing moiety comprises Formula VI

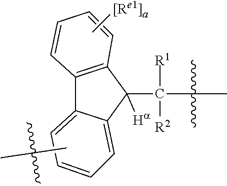

wherein Hα is an ionizable hydrogen atom; R¹ is H or an organic radical; R² is H or an organic radical; R^{e1}, when present, is a first electron altering group; (a) is either zero or one; the wavy line on the left indicates a bond to a polymer or a spacer moiety linked to a polymer and the wavy line on the right indicates a bond to a degradable linkage. In particular embodiments, (a) is zero.

In a further embodiment, the aromatic-containing moiety comprises Formula VII

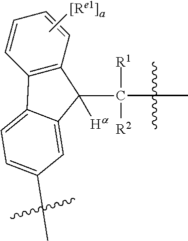

wherein Hα is an ionizable hydrogen atom; R¹ is H or an organic radical; R² is H or an organic radical; R^{e1}, when present, is a first electron altering group; (a) is either zero or one; the wavy line on the left indicates a bond to a polymer or a spacer moiety linked to a polymer and the wavy line on the right indicates a bond to a degradable linkage. In particular embodiments, (a) is zero.

In further embodiments, R^{e1} and R^{e2} above are each independently selected from the group consisting of halo, lower alkyl, aryl, substituted aryl, substituted arylakyl, alkoxy, aryloxy, alkylthio, arylthio, CF₃, —CH₂CF₃, —CH₂C₆F₅, —CN, —NO₂, —S(O)R, —S(O)Ar, —S(O₂)R, —S(O₂)Ar, —S(O₂)OR, —S(O₂)OAr, —S(O₂)NHR, —S(O₂)NHAr, —C(O)R, —C(O)Ar, —C(O)OR, and —C(O)NHR, wherein Ar is aryl and R is H or an organic radical.

In a further embodiment, the aromatic-containing moiety comprises Formula VIII

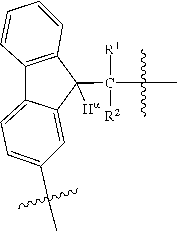

wherein Hα is an ionizable hydrogen atom; R¹ is H or an organic radical; R² is H or an organic radical; the wavy line on the left indicates a bond to a polymer or a spacer moiety linked to a polymer and the wavy line on the right indicates a bond to a degradable linkage.

In a further embodiment, the aromatic-containing moiety comprises Formula IX

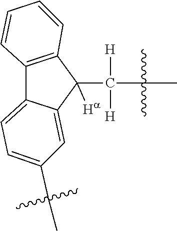

wherein Hα is an ionizable hydrogen atom; the wavy line on the left indicates a bond to a polymer or a spacer moiety linked to a polymer and the wavy line on the right indicates a bond to a degradable linkage.

### [Reactive Group]-[Degradable Linkage]-[Aromatic-Containing Moiety]-[Polymer]

In an exemplary embodiment, the [Reactive Group]-[Degradable Linkage]-[Aromatic-containing moiety]-[polymer] comprises Formula X:

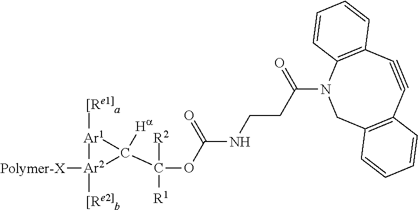

wherein polymer is a nonpeptidic, water-soluble polymer; Ar¹ is a first aromatic moiety; Ar² is a second aromatic moiety; X is a spacer moiety; Hα is an ionizable hydrogen atom; R¹ is H or an organic radical; R² is H or an organic radical; R^{e1}, when present, is a first electron altering group; R^{e2}, when present, is a second electron altering group; (a) is either zero or one; and (b) is either zero or one. In particular embodiments, (a) is zero and (b) is zero; (a) is one and (b) is zero; or (a) is zero and (b) is one.

In a further embodiment, the aromatic-containing moiety comprises Formula XI

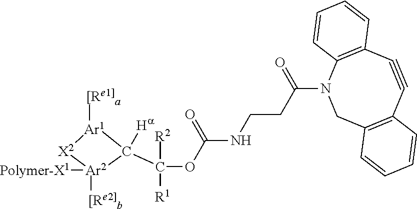

wherein Ar¹ is a first aromatic moiety; Ar² is a second aromatic moiety; Hα is an ionizable hydrogen atom; R¹ is H or an organic radical; R² is H or an organic radical; X¹ is a spacer moiety; X² is a spacer moiety; R^{e1}, when present, is a first electron altering group; R^{e2}, when present, is a second electron altering group; (a) is either zero or one; and (b) is either zero or one. In particular embodiments, (a) is zero and (b) is zero; (a) is one and (b) is zero; or (a) is zero and (b) is one.

In a further embodiment, the aromatic-containing moiety comprises Formula XII

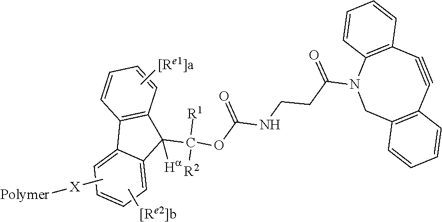

wherein Hα is an ionizable hydrogen atom; R¹ is H or an organic radical; R² is H or an organic radical; X s a spacer moiety; R^{e1}, when present, is a first electron altering group; R^{e2}, when present, is a second electron altering group; (a) is either zero or one; and (b) is either zero or one. In particular embodiments, (a) is zero and (b) is zero; (a) is one and (b) is zero; or (a) is zero and (b) is one.

In a further embodiment, the aromatic-containing moiety comprises Formula XIII

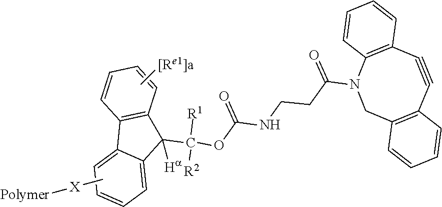

wherein Hα is an ionizable hydrogen atom; R¹ is H or an organic radical; R² is H or an organic radical; X is a spacer moiety; R^{e1}, when present, is a first electron altering group; and (a) is either zero or one. In particular embodiments, (a) is zero.

In a further embodiment, the aromatic-containing moiety comprises Formula XIV

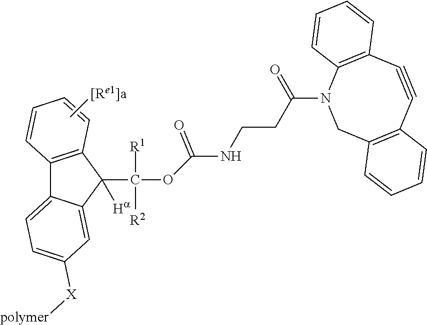

wherein Hα is an ionizable hydrogen atom; R¹ is H or an organic radical; R² is H or an organic radical; X is a spacer element; R^{e1}, when present, is a first electron altering group; and (a) is either zero or one. In particular embodiments, (a) is zero.

In further embodiments, R^{e1} and R^{e2} above are each independently selected from the group consisting of halo, lower alkyl, aryl, substituted aryl, substituted arylakyl, alkoxy, aryloxy, alkylthio, arylthio, CF₃, —CH₂CF₃, —CH₂C₆F₅, —CN, —NO₂, —S(O)R, —S(O)Ar, —S(O₂)R, —S(O₂)Ar, —S(O₂)OR, —S(O₂)OAr, —S(O₂)NHR, —S(O₂)NHAr, —C(O)R, —C(O)Ar, —C(O)OR, and —C(O)NHR, wherein Ar is aryl and R is H or an organic radical.

In a further embodiment, the aromatic-containing moiety comprises Formula XV

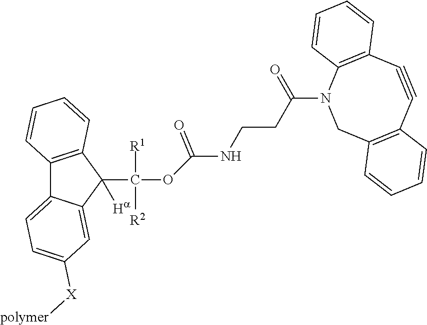

wherein Hα is an ionizable hydrogen atom; R¹ is H or an organic radical; R² is H or an organic radical; and X is a spacer moiety.

In a further embodiment, the aromatic-containing moiety comprises Formula XVI

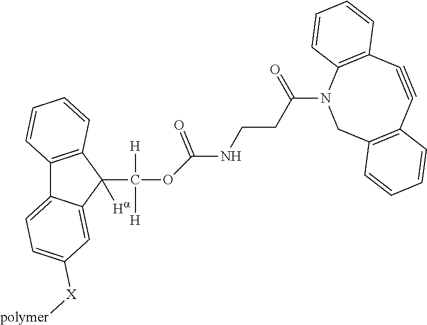

wherein Hα is an ionizable hydrogen atom; and X is a spacer moiety.

### [NNAA]-[Divalent Residue]-[Degradable Linkage]-[Aromatic-Containing Moiety]-[Polymer]

In an exemplary embodiment, the [NNAA]-[Divalent Residue]-[Degradable Linkage]-[Aromatic-containing moiety]-[polymer] comprises Formula XVII:

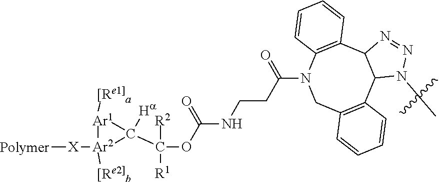

or regioisomer

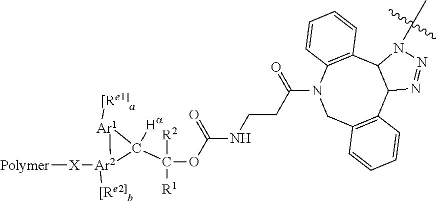

wherein polymer is a nonpeptidic, water-soluble polymer; Ar¹ is a first aromatic moiety; Ar² is a second aromatic moiety; X is a spacer moiety; Hα is an ionizable hydrogen atom; R¹ is H or an organic radical; R² is H or an organic radical; R^{e1}, when present, is a first electron altering group; R^{e2}, when present, is a second electron altering group; (a) is either zero or one; (b) is either zero or one; and the wavy line indicates a bond to the NNAA. In particular embodiments, (a) is zero and (b) is zero; (a) is one and (b) is zero; or (a) is zero and (b) is one.

In a further embodiment, the aromatic-containing moiety comprises Formula XVIII

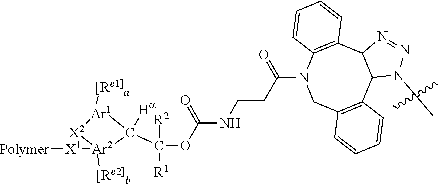

or regioisomer

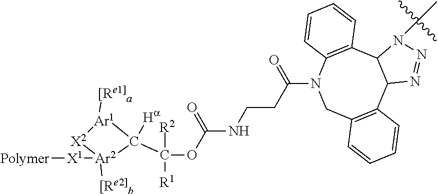

wherein Ar¹ is a first aromatic moiety; Ar² is a second aromatic moiety; Hα is an ionizable hydrogen atom; R¹ is H or an organic radical; R² is H or an organic radical; X¹ is spacer moiety; X² is a spacer moiety; R^{e1}, when present, is a first electron altering group; R^{e2}, when present, is a second electron altering group; (a) is either zero or one; (b) is either zero or one; and the wavy line indicates a bond to the NNAA. In particular embodiments, (a) is zero and (b) is zero; (a) is one and (b) is zero; or (a) is zero and (b) is one.

In a further embodiment, the aromatic-containing moiety comprises Formula XIX

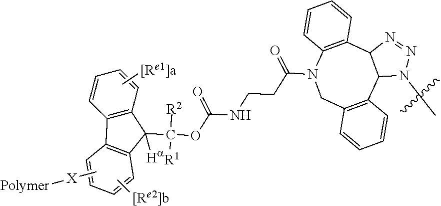

or regioisomer

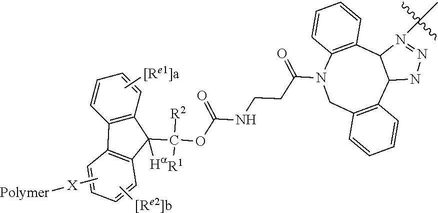

wherein Hα is an ionizable hydrogen atom; R¹ is H or an organic radical; R² is H or an organic radical; X s a spacer moiety; R^{e1}, when present, is a first electron altering group; R^{e2}, when present, is a second electron altering group; (a) is either zero or one; (b) is either zero or one; and the wavy line indicates a bond to the NNAA. In particular embodiments, (a) is zero and (b) is zero; (a) is one and (b) is zero; or (a) is zero and (b) is one.

In a further embodiment, the aromatic-containing moiety comprises Formula XX

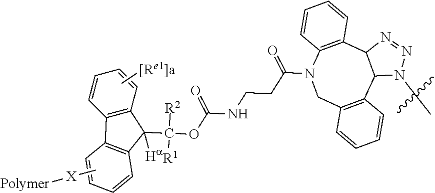

or regioisomer

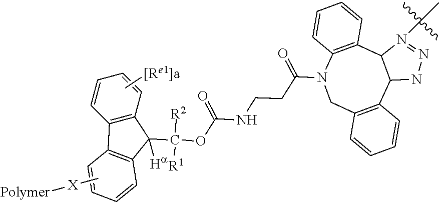

wherein Hα is an ionizable hydrogen atom; R¹ is H or an organic radical; R² is H or an organic radical; X is a spacer moiety; R^{e1}, when present, is a first electron altering group; (a) is either zero or one; the wavy line indicates a bond to the NNAA. In particular embodiments, (a) is zero.

In a further embodiment, the aromatic-containing moiety comprises Formula XXI

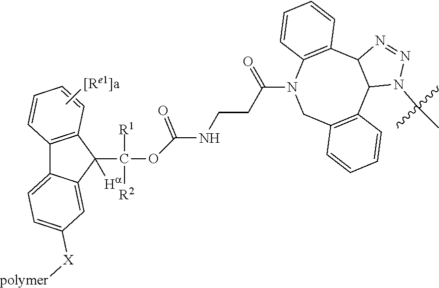

or regioisomer

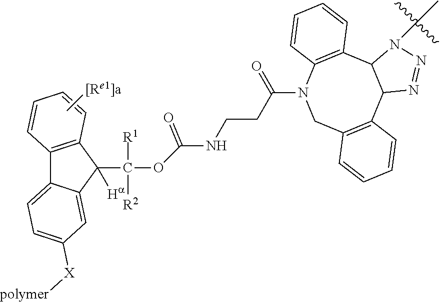

wherein Hα is an ionizable hydrogen atom; R¹ is H or an organic radical; R² is H or an organic radical; X is a spacer element; R^{e1}, when present, is a first electron altering group; (a) is either zero or one; and the wavy line indicates a bond to the NNAA. In particular embodiments, (a) is zero.

In further embodiments, R^{e1} and R^{e2} above are each independently selected from the group consisting of halo, lower alkyl, aryl, substituted aryl, substituted arylakyl, alkoxy, aryloxy, alkylthio, arylthio, CF₃, —CH₂CF₃, —CH₂C₆F₅, —CN, —NO₂, —S(O)R, —S(O)Ar, —S(O₂)R, —S(O₂)Ar, —S(O₂)OR, —S(O₂)OAr, —S(O₂)NHR, —S(O₂)NHAr, —C(O)R, —C(O)Ar, —C(O)OR, and —C(O)NHR, wherein Ar is aryl and R is H or an organic radical.

In a further embodiment, the aromatic-containing moiety comprises Formula XXII

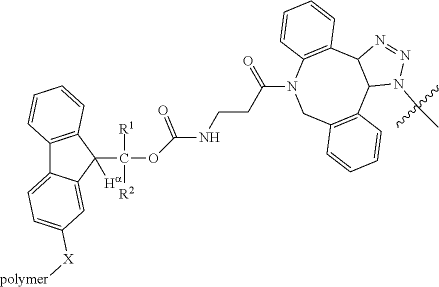

or regioisomer

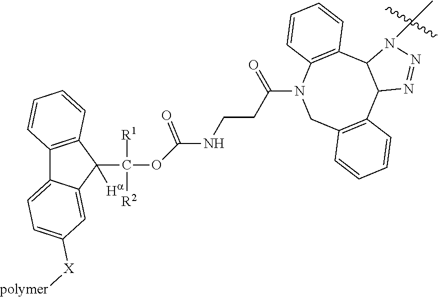

wherein Hα is an ionizable hydrogen atom; R¹ is H or an organic radical; R² is H or an organic radical; X is a spacer moiety; and the wavy line indicates a bond to the NNAA.

In a further embodiment, the aromatic-containing moiety comprises Formula XXIII

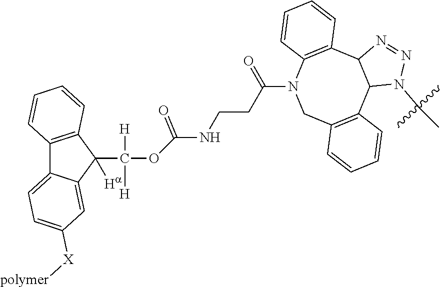

or regioisomer

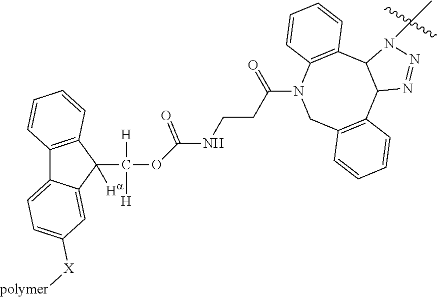

wherein Hα is an ionizable hydrogen atom; X is a spacer moiety; and the wavy line indicates a bond to the NNAA.
[pAMF within IL-2 Conjugate]-[Divalent Residue]-[Degradable Linkage]-[Aromatic-Containing Moiety]-[Polymer]

In an exemplary embodiment, the [pAMF within IL-2 conjugate]-[Divalent Residue]-[Degradable Linkage]-[Aromatic-containing moiety]-[polymer] comprises Formula XXIV:

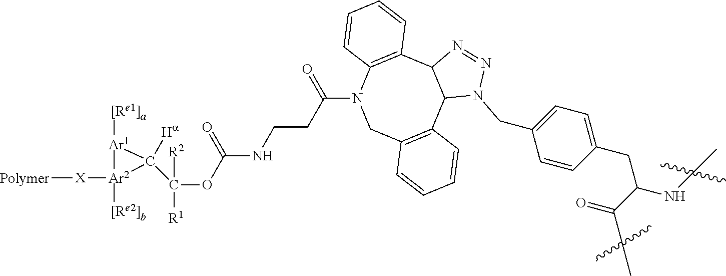

or regioisomer

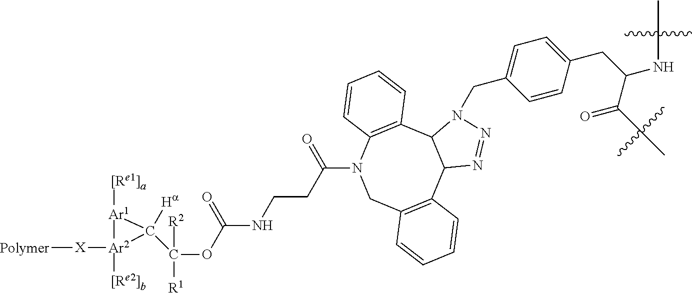

wherein polymer is a nonpeptidic, water-soluble polymer; Ar¹ is a first aromatic moiety; Ar² is a second aromatic moiety; X is a spacer moiety; Hα is an ionizable hydrogen atom; R¹ is H or an organic radical; R² is H or an organic radical; R^{e1}, when present, is a first electron altering group; R^{e2}, when present, is a second electron altering group; (a) is either zero or one; (b) is either zero or one; and the wavy lines indicate bonds to adjacent amino acids in the IL-2 conjugate. In particular embodiments, (a) is zero and (b) is zero; (a) is one and (b) is zero; or (a) is zero and (b) is one.

In a further embodiment, the aromatic-containing moiety comprises Formula XXV

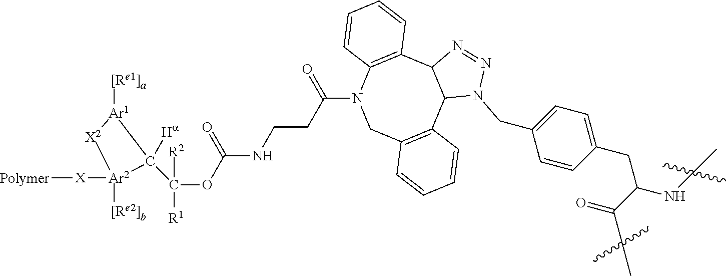

or regioisomer

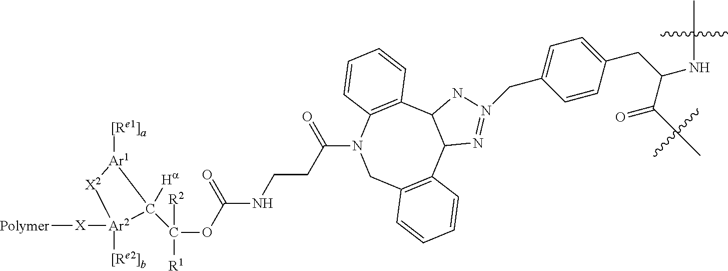

wherein Ar¹ is a first aromatic moiety; Ar² is a second aromatic moiety; Hα is an ionizable hydrogen atom; R¹ is H or an organic radical; R² is H or an organic radical; X¹ is spacer moiety; X² is a spacer moiety; R^{e1}, when present, is a first electron altering group; R^{e2}, when present, is a second electron altering group; (a) is either zero or one; (b) is either zero or one; and the wavy lines indicate bonds to adjacent amino acids in the IL-2 conjugate. In particular embodiments, (a) is zero and (b) is zero; (a) is one and (b) is zero; or (a) is zero and (b) is one.

In a further embodiment, the aromatic-containing moiety comprises Formula XXVI

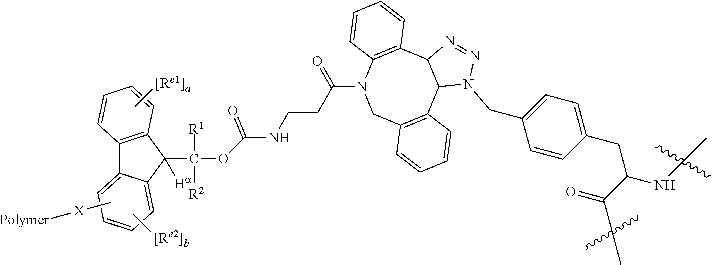

or regioisomer

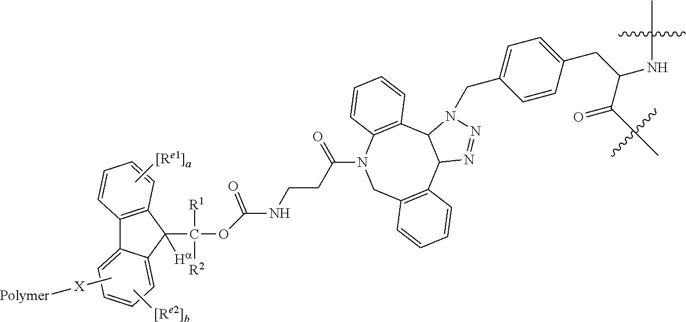

wherein Hα is an ionizable hydrogen atom; R¹ is H or an organic radical; R² is H or an organic radical; X s a spacer moiety; R^{e1}, when present, is a first electron altering group; R^{e2}, when present, is a second electron altering group; (a) is either zero or one; (b) is either zero or one; and the wavy lines indicate bonds to adjacent amino acids in the IL-2 conjugate. In particular embodiments, (a) is zero and (b) is zero; (a) is one and (b) is zero; or (a) is zero and (b) is one.

In a further embodiment, the aromatic-containing moiety comprises Formula XXVII

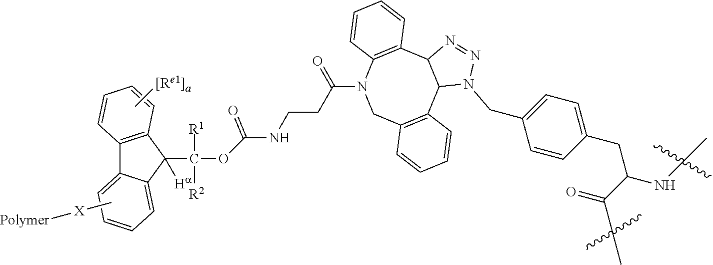

or regioisomer

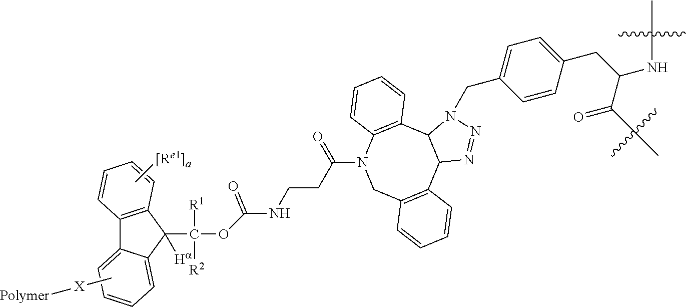

wherein Hα is an ionizable hydrogen atom; R¹ is H or an organic radical; R² is H or an organic radical; X is a spacer moiety; R^{e1}, when present, is a first electron altering group; (a) is either zero or one; and the wavy lines indicate bonds to adjacent amino acids in the IL-2 conjugate. In particular embodiments, (a) is zero.

In a further embodiment, the aromatic-containing moiety comprises Formula XXVIII

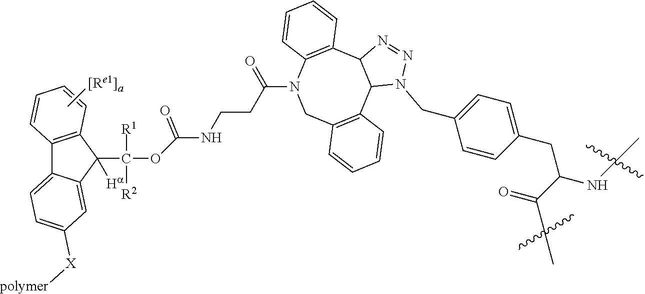

or regioisomer

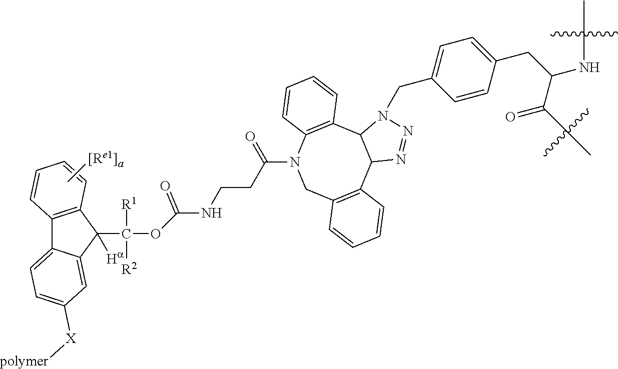

wherein Hα is an ionizable hydrogen atom; R¹ is H or an organic radical; R² is H or an organic radical; X is a spacer element; R^{e1}, when present, is a first electron altering group; (a) is either zero or one; and the wavy lines indicate bonds to adjacent amino acids in the IL-2 conjugate. In particular embodiments, (a) is zero.

In further embodiments, R^{e1} and R^{e2} above are each independently selected from the group consisting of halo, lower alkyl, aryl, substituted aryl, substituted arylakyl, alkoxy, aryloxy, alkylthio, arylthio, CF₃, —CH₂CF₃, —CH₂C₆F₅, —CN, —NO₂, —S(O)R, —S(O)Ar, —S(O₂)R, —S(O₂)Ar, —S(O₂)OR, —S(O₂)OAr, —S(O₂)NHR, —S(O₂)NHAr, —C(O)R, —C(O)Ar, —C(O)OR, and —C(O)NHR, wherein Ar is aryl and R is H or an organic radical.

In a further embodiment, the aromatic-containing moiety comprises Formula XXIX

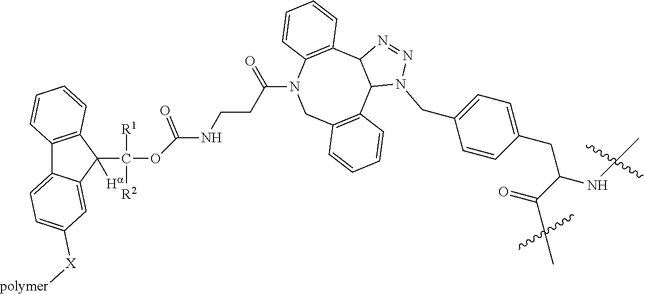

or regioisomer

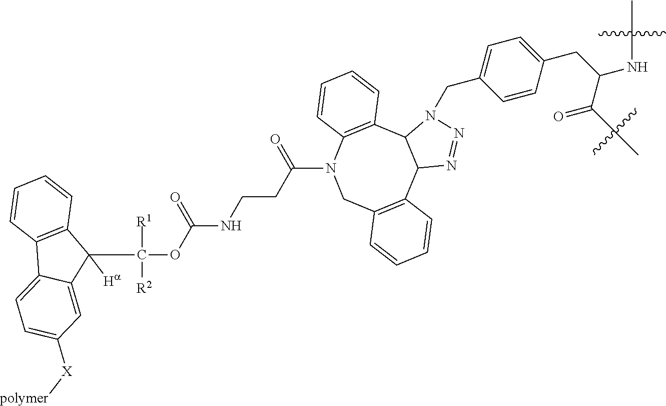

wherein Hα is an ionizable hydrogen atom; R¹ is H or an organic radical; R² is H or an organic radical; X is a spacer moiety; and the wavy lines indicate bonds to adjacent amino acids in the IL-2 conjugate.

In a further embodiment, the aromatic-containing moiety comprises Formula XXX

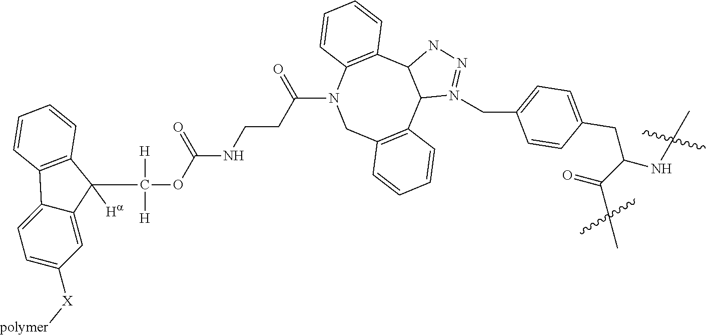

or regioisomer

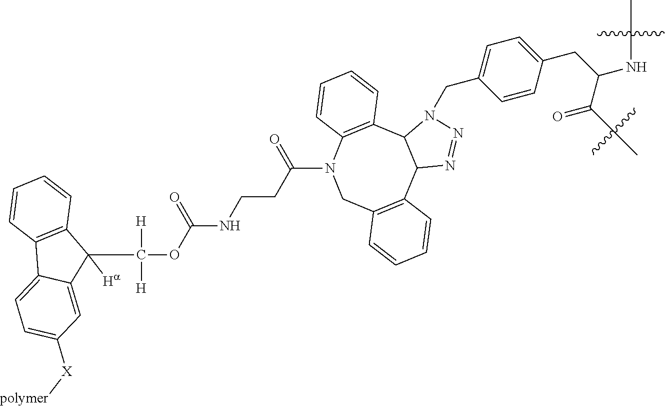

wherein Hα is an ionizable hydrogen atom; X is a spacer moiety; the wavy line on the left indicates a bond to a polymer or a spacer moiety linked to a polymer and the wavy lines indicate bonds to adjacent amino acids in the IL-2 conjugate.

### Nonnatural Amino Acids

The nonnatural amino acid incorporated into the IL-2 moiety comprising the IL-2 prodrug of the present invention may be any nonnatural amino acid deemed suitable by the practitioner for conjugating a nonpeptidic, water-soluble polymer thereto. In general, the nonnatural amino acid comprises a functional group useful for forming a covalent bond to a reactive group present within a nonpeptidic, water-soluble polymer or on a linker linked to the nonpeptidic, water-soluble polymer. In certain embodiments, the functional group is selected from the group consisting of amino, carboxy, acetyl, hydrazino, hydrazido, semicarbazido, sulfanyl, azido, and alkynyl with the proviso that the functional group is selected as being capable of forming a covalent bond with the reactive group within or linked to the nonpeptidic, water-soluble polymer. Suitable modified amino acids include those described in, for example, WO2013185115 and WO2015006555, each of which is incorporated herein by reference in its entirety.

In certain embodiments, the amino acid residue is according to any of the following formulas:

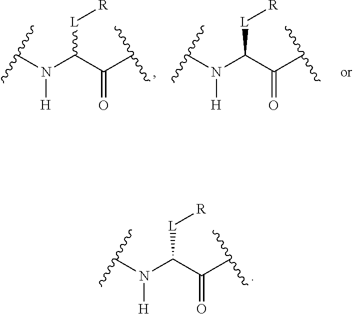

In the above formulas, the wavy lines indicate bonds that connect to the remainder of the polypeptide chains of the IL-2 prodrug. These non-natural amino acids can be incorporated into polypeptide chains just as natural amino acids are incorporated into the same polypeptide chains. In certain embodiments, the non-natural amino acids are incorporated into the polypeptide chain via amide bonds as indicated in the formulas.

In the above formulas, R designates any functional group without limitation, so long as the amino acid residue is not identical to a natural amino acid residue. In certain embodiments, R can be a hydrophobic group, a hydrophilic group, a polar group, an acidic group, a basic group, a chelating group, a reactive group, a therapeutic moiety, or a labeling moiety. In certain embodiments, R is selected from the group consisting of: R¹NR²R³, R¹C(—O) R², R¹C(—O)OR², R¹N₃, and R¹C(═CH). In these embodiments, R¹ is selected from the group consisting of: a bond, alkylene, heteroalkylene, arylene, and heteroarylene. R² and R³ are each independently selected from the group consisting of: hydrogen, alkyl and heteroalkyl. Those of skill in the art will recognize that proteins are generally comprised of L-amino acids. However, the present methods and compositions provide the practitioner with the ability to use L, D, or racemic nonnatural amino acids at the site specific positions. In certain embodiments, the nonnatural amino acids described herein include D-versions of the natural amino acids and racemic versions of the natural amino acids.

In the above formulas, each L represents an optional divalent linker. The divalent linker can be any divalent linker known to those of skill in the art. Generally, the divalent linker is capable of forming covalent bonds to the functional moiety R and the alpha carbon of the non-natural amino acid. Useful divalent linkers, include an alkylene, substituted alkylene, heteroalkylene, substituted heteroalkylene, arylene, substituted arylene, heteroarylene and substituted heteroarylene. In certain embodiments, L is C₁₋₁₀ alkylene or C₁₋₁₀ heteroalkylene.

The site-specific nonnatural amino acids include a side chain functional group that reacts efficiently and selectively with reactive groups and are not found in the 20 common amino acids. The side chain functional group forms a stable conjugate with the reactive group of the nonpeptidic, water-soluble polymer. Examples of side chain functional groups include but are not limited to azido, ketone, aldehyde and aminooxy groups. For example, the IL-2 moiety that includes a site-specific nonnatural amino acid containing an azido functional group can be reacted with a reactive group of a nonpeptidic, water-soluble polymer containing an alkyne moiety to form a prodrug having a stable conjugate resulting from the selective reaction of the azide and the alkyne groups to form a Huisgen [3+2] cycloaddition product.

Exemplary site-specific nonnatural amino acids that may be suitable for use in the present invention and that are useful for reactions with nonpeptidic water-soluble polymers include, but are not limited to, those with carbonyl, aminooxy, hydrazine, hydrazide, semicarbazide, azide, and alkyne functional groups. In some embodiments, site-specific nonnatural amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, athioether, an amide and the like. Examples of such amino acids also include saccharides that are not commonly found in naturally-occurring proteins such as 2-deoxy-glucose, 2-deoxygalactose and the like.

Specific examples of unnatural amino acids that may be suitable for use in the present invention include, but are not limited to, p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine, and the like. Examples of structures of a variety of unnatural amino acids that may be suitable for use in the present invention are provided in, for example, WO 2002085923 entitled “In vivo incorporation of unnatural amino acids.” See also Kiick et al., (2002) Incorporation of azides into recombinant proteins for chemoselective modification by the Staudinger ligation, PNAS 99:19-24, for additional methionine analogs.

Many of the nonnatural amino acids suitable for use in the present invention are commercially available, e.g., from Sigma-Aldrich (Milwaukee, WI). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO2002/085923; Matsoukas et al., J. Med. Chem., 38, 4660-4669 (1995); King & Kidd, J. Chem. Soc., 3315-3319 (1949); Friedman & Chatterrji, J. Am. Chem. Soc. 81, 3750-3752 (1959); Craig et al. J. Org. Chem. 53, 1167-1170 (1988); Azoulay et al., Eur. J. Med. Chem. 26, 201-5 (1991); Koskinen & Rapoport, J. Org. Chem. 54, 1859-1866 (1989); Christie & Rapoport J. Org. Chem. 1989:1859-1866 (1985); Barton et al., Tetrahedron Lett. 43:4297-4308 (1987); and, Subasinghe et al., J. Med. Chem. 35:4602-7 (1992). See also, U.S. Pat. No. 10,669,347, in which the content therein from column 21, line 37, to column 81, line 23, disclosing nonnatural amino acids are incorporated herein by reference in its entirety.

The unique reactivity of azide and alkyne groups makes such groups extremely useful for the selective modification of polypeptides and other biological molecules. Organic azides, particularly aliphatic azides, and alkynes are generally stable toward common reactive chemical conditions. In particular, both the azide and the alkyne functional or reactive groups are inert toward the side chains (i.e., R groups) of the 20 common amino acids found in naturally-occurring polypeptides. When brought into close proximity, the “spring-loaded” nature of the azide and alkyne groups is revealed, and they react selectively and efficiently via Huisgen [3+2] cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the Huisgen cycloaddition reaction involves a selective cycloaddition reaction (see, e.g., Padwa, A., in Comprehensive Organic Synthesis, Vol. 4, (ed. Trost, B. M., 1991), p. 1069-1109; Huisgen, R. in 1,3-Dipolar Cycloaddition Chemistry, (ed. Padwa, A., 1984), p. 1-176) rather than a nucleophilic substitution, the incorporation of site-specific nonnatural amino acids bearing azide and alkyne-containing side chains permits the resultant polypeptides to be modified selectively at the position of the site-specific nonnatural amino acid. Cycloaddition reaction involving azide or alkyne-containing protein can+be carried out at room temperature under aqueous conditions by the addition of Cu(II) (including but not limited to, in the form of a catalytic amount of CuSO₄) in the presence of a reducing agent for reducing Cu(II) to Cu(I), in situ, in catalytic amount. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, but are not limited to, ascorbate, metallic copper, quinine, hydroquinone, vitamin K, glutathione, cysteine, Fe² Co²⁺, and an applied electric potential.

In some cases, where a Huisgen [3+2] cycloaddition reaction between an azide and an alkyne is desired, the antigen-binding polypeptide comprises a site-specific nonnatural amino acid comprising an alkyne moiety and the water-soluble polymer to be attached to the amino acid comprises an azide moiety. Alternatively, the converse reaction (i.e., with the azide moiety on the amino acid and the alkyne moiety present on the nonpeptidic, water-soluble polymer) can also be performed.

The azide functional group can also be reacted selectively with a nonpeptidic, water-soluble polymer containing an aryl ester and appropriately functionalized with an aryl phosphine moiety to generate an amide linkage. The aryl phosphine group reduces the azide in situ and the resulting amine then reacts efficiently with a proximal ester linkage to generate the corresponding amide. See, e.g., E. Saxon and C. Bertozzi, Science 287, 2007-2010 (2000). The azide-containing amino acid can be either an alkyl azide (including but not limited to, 2-amino-6-azido-l-hexanoic acid) or an aryl azide (p-azido-phenylalanine).

Exemplary azide-containing amino acids include the following:

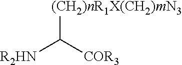

wherein n is 0-10; R₁ is an alkyl, aryl, substituted alkyl, substituted aryl or not present; X is O, N, S, or not present; m is 0-10; R₂ is H, an amino acid, a polypeptide, or an amino terminus modification group, and R³ is H, an amino acid, a polypeptide, or a carboxy terminus modification group. In some embodiments, n is 1, R₁ is phenyl, X is not present, m is 0 and the azide moiety is positioned para to the alkyl side chain. In some embodiments, n is 0-4 and R₁ and X are not present, and m is 0. In some embodiments, n is 1, R₁ is phenyl, X is 0, m is 2 and the p-azidoethoxy moiety is positioned in the para position relative to the alkyl side chain.

Azide-containing amino acids are available from commercial sources. For instance, 4-azidophenylalanine can be obtained from Chem-Impex International, Inc. (Wood Dale, Ill.). For those azide-containing amino acids that are not commercially available, the azide group can be prepared relatively readily using standard methods known to those of skill in the art, including but not limited to, via displacement of a suitable leaving group (including but not limited to, halide, mesylate, tosylate) or via opening of a suitably protected lactone. See, e.g., Advanced Organic Chemistry by March (Third Edition, 1985, Wiley and Sons, New York).

In certain embodiments, the nonnatural amino acid is according to the following formula:

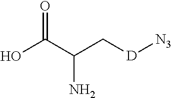

or a salt thereof, wherein: D is —Ar—W₃— or —W₁—Y₁—C(O)—Y₂—W₂—; Ar is

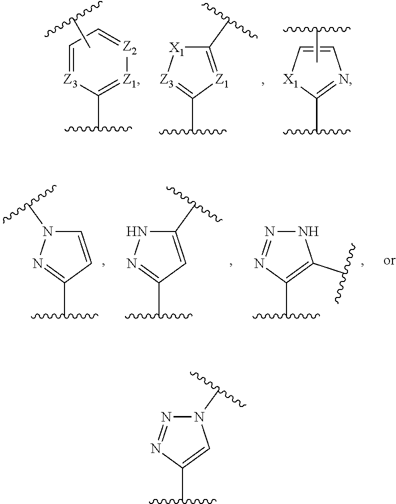

each of W₁, W₂, and W₃ is independently a single bond or lower alkylene; each X¹ is independently —NH—, —O—, or —S—; each Y₁ is independently a single bond, —NH—, or —O—; each Y₂ is independently a single bond, —NH—, —O—, or an N-linked or C-linked pyrrolidinylene; and one of Z₁, Z₂, and Z₃ is —N— and the others of Z₁, Z₂, and Z₃ are independently —CH—, and wherein the wavy line indicates a bond to an adjacent atom.

In certain embodiments, the nonnatural amino acid is according to the following formula:

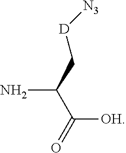

where D is —Ar—W₃— or —W₁—Y₁—C(O)—Y₂—W₂—. In certain embodiments, the nonnatural amino acid is according the following formula:

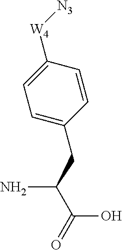

or a salt thereof, wherein W₄ is C₁-C₁₀ alkylene. In a further embodiment, W₄ is C₁-C₅ alkylene. In an embodiment, W₄ is C₁-C₃ alkylene. In an embodiment, W₄ is C₁ alkylene.

In particular embodiments, the nonnatural amino acid may be p-azidomethylphenylalanine (pAMF):

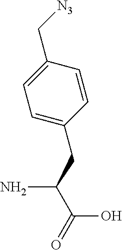 [SMILES: [N-]=[N+]=NCc1ccc(C[C@H](N)C(=O)O)cc1]

or a salt thereof. Such nonnatural amino acids may be in the form of a salt or may be incorporated into a nonnatural amino acid polypeptide, polymer, polysaccharide, or a polynucleotide and optionally post translationally modified.

### Nonpeptidic, Water-Soluble Polymers

The releasable linker further comprises a nonpeptidic, water-soluble polymer conjugated to the aromatic-containing moiety located at the distal end of the releasable linker and may be represented by the following formula
[Reactive Group]-[Degradable Linkage]-[Aromatic-containing moiety]-[Polymer].

With respect to the nonpeptidic, water-soluble polymer, in certain embodiments, the nonpeptidic, water-soluble polymer is nontoxic, non-naturally occurring, and biocompatible. With respect to biocompatibility, a substance is considered biocompatible if the beneficial effects associated with use of the substance alone or with another substance in connection with living tissues (e.g., administration to a patient) outweighs any deleterious effects as evaluated by a clinician, e.g., a physician. With respect to non-immunogenicity, a substance is considered non-immunogenic if the intended use of the substance in vivo does not produce an undesired immune response (e.g., the formation of antibodies) or, if an immune response is produced, that such a response is not deemed clinically significant or important as evaluated by a clinician. In specific embodiments, the nonpeptidic, water-soluble polymer is biocompatible and non-immunogenic.

Further, the nonpeptidic, water-soluble polymer is typically characterized as having from two to about 300 termini. Examples of such polymers include, but are not limited to, poly(alkylene glycols) such as polyethylene glycol (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxy-alkylmethacrylate), poly(saccharides), poly(a-hydroxy acid), poly(vinyl alcohol), polyphosphazene, polyoxazolines (“POZ”) (which are described m WO2008/106186), poly(N-acryloylmorpholine), and combinations of any of the foregoing.

The nonpeptidic, water-soluble polymer is not limited to a particular structure and can be linear (e.g., an end capped, e.g., alkoxy PEG such as methoxy PEG (mPEG) or a bifunctional PEG), branched or multi-armed (e.g., forked PEG or PEG attached to a polyol core), or a dendritic (or star) architecture. Moreover, the internal structure of the nonpeptidic, water-soluble polymer can be organized in any number of different repeat patterns and can be selected from the group consisting of homopolymer, alternating copolymer, random copolymer, block copolymer, alternating tripolymer, random tripolymer, and block tripolymer.

Typically, activated PEG and other activated nonpeptidic, water-soluble polymers (i.e., polymeric reagents) are activated with a suitable reactive group appropriate for coupling to a functional group on the aromatic-containing polymer. Representative polymeric reagents and methods for conjugating these polymers to an active moiety are known in the art and further described in Zalipsky, S., et al., “Use of Functionalized Poly (Ethylene Glycols) for Modification of Polypeptides” in Polyethylene Glycol Chemistry: Biotechnical and Biomedical Applications, J. M. Harris, Plenus Press, New York (1992), and in Zalipsky Advanced Drug Reviews 16:157-182 (1995). Exemplary reactive groups suitable for coupling to a functional group of an aromatic-containing polymer include hydroxyl, maleimide, ester, acetal, ketal, amine, carboxyl, aldehyde, aldehyde hydrate, ketone, vinyl ketone, thione, thiol, vinyl sulfone, hydrazine, alkyne, azide, among others. In particular embodiments, the reactive group is an amine and the functional group is a carboxyl group.

Typically, the weight-average molecular weight of the nonpeptidic, water-soluble polymer in the conjugate is from about one kiloDaltons (kDa) to about 150 kDa. Exemplary ranges, however, include weight-average molecular weights in the range of greater than 5 kDa to about 100 kDa, in the range of from about 6 kDa to about 90 kDa, in the range of from about 10 kDa to about 85 kDa, in the range of greater than 10 kDa to about 85 kDa, in the range of from about 20 kDa to about 85 kDa, in the range of from about 53 kDa to about 85 kDa, in the range of from about 25 kDa to about 120 kDa, in the range of from about 29 kDa to about 120 kDa, in the range of from about 35 kDa to about 120 kDa, and in the range of from about 40 kDa to about 120 kDa. For any given nonpeptidic, water-soluble polymer, PEGs having a molecular weight in one or more of these ranges are preferred.

Exemplary weight-average molecular weights for the nonpeptidic, water-soluble polymer include about 1 kDa, about 1.5 kDa, about 2 kDa, about 2.2 kDa, about 2.5 kDa, about 3 kDa, about 4 kDa, about 4.5 kDa, about 5 kDa, about 5.5 kDa, about 6 kDa, about 7 kDa, about 7.5 kDa, about 8 kDa, about 9 kDa, about 10 kDa, about 11 kDa, about 12 kDa, about 13 kDa, about 14 kDa, about 15 kDa, about 20 kDa, about 22.5 Da, about 25 kDa, about 30 kDa, about 35 kDa, about 40 kDa, about 45 kDa, about 50 kDa, about 55 kDa, about 60 kDa, about 65 kDa, about 70 kDa, and about 75 kDa. Branched versions of the nonpeptidic, water-soluble polymer (e.g., a branched 40 kDa nonpeptidic, water-soluble polymer comprised of two 20 kDa polymers, or a branched 20 kDa nonpeptidic, water-soluble polymer comprised of two 10 kDa polymers) having a total molecular weight of any of the foregoing can also be used.

PEGs typically comprise a number of (OCH₂CH₂) monomers (or (CH₂CH₂O) monomers, depending on how the PEG is defined). As used throughout the description, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

In one embodiment, the polymer for use herein is an end-capped polymer, that is, a polymer having at least one terminus capped with a relatively inert group, such as a lower C₁₋₆ alkoxy group, although a hydroxyl group can also be used. When the polymer is PEG, for example, it may be desirable to use a methoxy-PEG (commonly referred to as mPEG), which is a linear form of PEG wherein one terminus of the polymer is a methoxy (—OCH₃) group, while the other terminus is a hydroxyl or other reactive group that can be optionally chemically modified.

In certain embodiments, free or unbound PEG is a linear polymer terminated at each end with hydroxyl groups: HO—CH₂CH₂O—(CH₂CH₂O)ₙ—CH₂CH₂—OH, wherein (n) typically ranges from zero to about 4,000. The above polymer, alpha-, omega-dihydroxylpoly(ethylene glycol), can be represented in brief form as HO-PEG-OH where it is understood that the -PEG- symbol can represent the following structural unit: —CH₂CH₂O—(CH₂CH₂O)ₙ—CH₂CH₂—, wherein (n) is as defined as above.

Another type of PEG useful in one or more embodiments is methoxy-PEG-OH, or mPEG in brief, in which one terminus is the relatively inert methoxy group, while the other terminus is a hydroxyl group. The formula of mPEG is given below. CH₃O—CH₂CH₂O—(CH₂CH₂O)ₙ—CH₂CH₂—OH wherein (n) is as described above.

Multi-armed or branched PEG molecules, such as those described in U.S. Pat. No. 5,932,462, can also be used as the PEG polymer. For example, PEG can have the formula:

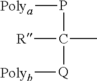

wherein polyₐ and poly_{b} are PEG backbones (either the same or different), such as methoxy poly(ethylene glycol); R″ is a nonreactive moiety, such as H, methyl, or a PEG backbone; and P and Q are nonreactive linkages.

In addition, the PEG can comprise a forked PEG. An example of a forked PEG is represented by the following formula:

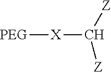

wherein X is a linker of one or more atoms and each Z is an activated terminal group linked to CH by a chain of atoms of defined length. International Patent Application Publication WO9945964 discloses various forked PEG structures capable of use in one or more embodiments of the present invention. The chain of atoms linking the Z functional groups to the branching carbon atom serve as a tethering group and may comprise, for example, alkyl chains, ether chains, ester chains, amide chains and combinations thereof.

The PEG polymer may comprise a pendant PEG molecule having reactive groups, such as carboxyl, covalently attached along the length of the PEG rather than at the end of the PEG chain. The pendant reactive groups can be attached to the PEG directly or through a linker, such as an alkylene group.

Those of ordinary skill in the art will recognize that the foregoing discussion concerning nonpeptidic, water-soluble polymer is by no means exhaustive and is merely illustrative, and that all polymeric materials having the qualities described above are contemplated.

The attachment between the aromatic-containing moiety and the nonpeptidic, water-soluble polymer can be direct, wherein no intervening atoms are located between the aromatic-containing moiety and the nonpeptidic, water-soluble polymer, or indirect, wherein a spacer moiety comprising one or more atoms is located between the aromatic-containing moiety and the nonpeptidic, water-soluble polymer. The one or more atoms making up the spacer moiety can include one or more of carbon atoms, nitrogen atoms, sulfur atoms, oxygen atoms, and combinations thereof. The spacer moiety can comprise an amide, secondary amine, carbamate, thioether, and/or disulfide group. Nonlimiting examples of specific spacer moieties include those selected from the group consisting of —O—, —S—, —S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—N—H—C(O)—CH₂—CH₂—, —O—C(O)—NH—[CH₂]ₕ—(OCH₂CH₂)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (J) is zero to 20. Other specific spacer moieties have the following formulas: —C(O)—NH—(CH₂)₁₋₆—NH—C(O)—NH—C(O)—NH—(CH₂)₁₋₆—NH—C(O)—, and —O—C(O)—NH—(CH₂)₁₋₆—NH—C(O)—, wherein the subscript values following each methylene indicate the number of methylene groups contained in the formula, e.g., (CH₂)₁₋₆ means that the formula can contain 1, 2, 3, 4, 5 or 6 methylene groups. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment. In particular embodiments, the spacer moiety comprises 1, 2, 3, 4,5, 6, 7, 8, 9, or 10 carbon atoms. Exemplary spacer moieties include —C(O)—.

Any molecular mass for a PEG can be used as practically desired, including but not limited to, from about 100 Da to 100 kDa or more as desired (including but not limited to, sometimes from about 0.1-50 kDa or about 10-40 kDa or about 20 kDa). Branched chain PEGs, including but not limited to, PEG molecules with each chain having a molecular weight (MW) ranging from 1-100 kDa (including but not limited to, 1-50 kDa or 5-20 kDa) can also be used. A wide range of PEG molecules are described in, including but not limited to, the Shearwater Polymers, Inc. catalog, and the Nektar Therapeutics catalog, incorporated herein by reference.

### Preparation of IL-2 Prodrugs

The IL-2 prodrugs of the present invention may be prepared by standard techniques. In certain embodiments, an IL-2 moiety is contacted with a nonpeptidic, water-soluble polymer precursor under conditions suitable for forming a covalent bond linking the IL-2 moiety to the nonpeptidic, water-soluble polymer precursor to form an IL-2 moiety-water-soluble polymer conjugate. IL-2 prodrugs comprise those embodiments of the IL-2 moiety-water-soluble polymer conjugate that display substantially attenuated or no detectable binding activity at the IL-2α and IL-2β receptors as determined by surface plasmon resonance.

In certain embodiments, an IL-2 moiety is contacted with a linker precursor under conditions suitable for forming a covalent bond from the IL-2 moiety to the linker. The resulting IL-2 moiety-linker is contacted with a nonpeptidic, water-soluble polymer precursor under conditions suitable for forming a covalent bond from the IL-2 moiety-linker to the nonpeptidic, water-soluble polymer precursor to form an IL-2 moiety-linker-nonpeptidic, water-soluble polymer conjugate. IL-2 prodrugs comprise those embodiments of the IL-2 moiety-linker-water-soluble polymer conjugate that display substantially attenuated or no detectable binding activity at the IL-2α and IL-2β receptors as determined by surface plasmon resonance.

In certain embodiments, a nonpeptidic, water-soluble polymer precursor is contacted with a linker precursor under conditions suitable for forming a covalent bond from the nonpeptidic, water-soluble polymer to the linker. The resulting nonpeptidic-water-soluble polymer-linker is contacted with an IL-2 moiety under conditions suitable for forming a covalent bond from the nonpeptidic, water-soluble polymer-linker to the IL-2 moiety to form an IL-2 moiety-linker-nonpeptidic, water-soluble polymer conjugate. IL-2 prodrugs comprise those embodiments of the IL-2 moiety-linker-nonpeptidic, water-soluble polymer conjugate that display substantially attenuated or no detectable binding activity at the IL-2α and IL-2β receptors as determined by surface plasmon resonance. Suitable linkers for preparing the IL-2 prodrugs are disclosed herein, and exemplary conditions for conjugation are described in the Examples below.

Embodiments are also directed to the provision of isolated nucleic acids encoding IL-2 moieties, vectors and host cells comprising the nucleic acids, and recombinant techniques for the production of IL-2 moieties.

For recombinant production of an IL-2 moiety, the nucleic acid(s) encoding it may be isolated and inserted into a replicable vector for further cloning (i.e., amplification of the DNA) or expression. In some embodiments, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Expression of the IL-2 moiety, which comprises one or more nonnatural amino acids, may be performed in an orthogonal biosynthetic translation system that is capable of site-specific substitution of any selected amino acid within the sequence of IL-2 with a nonnatural amino acid. Such orthogonal biosynthetic translational machinery comprises orthogonal tRNAs and orthogonal-RS (O-RS) and orthogonal tRNAs/O-RS pairs, which when introduced into a host cell or cell-free translation system, can be used to incorporate a nonnatural amino acid into a polypeptide (protein) of interest. The orthogonal tRNA delivers the nonnatural amino acid in response to a selector codon and the orthogonal synthetase preferentially aminoacylates an orthogonal tRNA with the nonnatural amino acid. The O-RS does not efficiently aminoacylate the orthogonal tRNA with any of the common twenty amino acids. Methods for constructing orthogonal biosynthetic translation system for cell-based or cell-free expression and using such systems for incorporating nonnatural amino acids into a polypeptide at predetermined sites are known in the art and have been disclosed, for example, U.S. Pat. Nos. 9,797,908; 7,736,872; 9,163,271; 9,7979,08; 9,797,908; 8,445,446; 7,736,872; 7,846,689; and US publication No. 20170292139; each of which is herein incorporated by reference in their entirety.

Once the IL-2 moiety incorporating the nonnatural amino acid(s) has been produced in the host cell or cell-free orthogonal translation system, it can be extracted therefrom by a variety of techniques known in the art, including enzymatic, chemical and/or osmotic lysis and physical disruption. The IL-2 moiety can be purified by standard techniques known in the art such as preparative chromatography, affinity purification or any other suitable technique.

Suitable host cells may include bacterial cells, for example E. coli, and eukaryote cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells that comprise an orthogonal translation system designed for production or proteins comprising nonnatural amino acids. Other suitable host cells are known to those skilled in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the vector encoding the IL-2 moiety described herein is integrated into the genome of the host cell. An advantage of stable integration is that the uniformity between individual cells or clones is achieved. Another advantage is that selection of the best producers may be performed. Accordingly, it is desirable to create stable cell lines. In another embodiment, the conjugates described herein are transfected into a host cell. An advantage of transfecting the conjugates into the host cell is that protein yields may be maximized. In one aspect, there is described a cell comprising the nucleic acid construct or the vector described herein.

### Pharmaceutical Compositions and Methods of Administration

The IL-2 prodrugs provided herein can be formulated into pharmaceutical compositions using methods available in the art and those disclosed herein. Any of the IL-2 prodrugs provided herein can be provided in the appropriate pharmaceutical composition and be administered by a suitable route of administration.

The methods provided herein encompass administering pharmaceutical compositions comprising at least one IL-2 prodrug provided herein and one or more compatible and pharmaceutically acceptable earners. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or IL-2 prodrugs provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition or IL-2 prodrug provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic IL-2 prodrugs.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific IL-2 moiety in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects of the present invention, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects of the present invention, the solvent is saline solution, such as a sterile isotonic saline solution or dextrose solution. In some aspects of the present invention, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects of the present invention, the microparticles or nanoparticles are micelles, liposomes, or polymersomes.

Further provided herein are anhydrous pharmaceutical compositions and dosage forms comprising an IL-2 prodrug, since, in some embodiments, water can facilitate the degradation of some proteins.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmacopeia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an IL-2 moiety or IL-2 prodrug will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the proteins disclosed herein can also be incorporated into the parenteral dosage forms.

### Dosage and Unit Dosage Forms

In human therapeutics, the doctor will determine the posology which he considers most appropriate according to a preventive or curative treatment and according to the age, weight, condition and other factors specific to the subject to be treated

In certain embodiments, a composition provided herein 1s a pharmaceutical composition or a single unit dosage form. Pharmaceutical compositions and single unit dosage forms provided herein comprise a prophylactically or therapeutically effective amount of one or more prophylactic or therapeutic proteins.

The amount of the IL-2 prodrug or composition which will be effective in the prevention or treatment of a disorder or one or more symptoms thereof will vary with the nature and severity of the disease or condition, and the route by which the IL-2 moiety is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the IL-2 moiety per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the IL-2 prodrug provided herein, based on weight of the IL-2 moiety, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight.

The dose can be administered according to a suitable schedule, for example, once, two times, three times, or four times weekly. It may be necessary to use dosages of the IL-2 prodrug outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the proteins provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an IL-2 prodrug or composition provided herein followed by one or more maintenance doses.

In certain embodiments, a dose of an IL-2 prodrug or composition provided herein can be administered to achieve a steady-state concentration of the IL-2 moiety in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### Therapeutic Applications

For therapeutic applications, the IL-2 prodrugs provided herein can be administered to a mammal, generally a human, in a pharmaceutically acceptable dosage form such as those known in the art and those discussed above. For example, the IL-2 prodrugs may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The IL-2 prodrugs also are suitably administered by peritumoral, intralesional, or perilesional routes, to exert local as well as systemic therapeutic effects. The intraperitoneal route may be particularly useful, for example, in the treatment of ovarian tumors.

The IL-2 prodrugs provided herein may be useful for the treatment of any disease or condition involving an IL2 receptor. In some embodiments, the disease or condition is a disease or condition that would benefit from stimulation or amplification of the immune response. In some embodiments, the disease or condition is a disease or condition that can benefit from treatment with an IL-2 moiety. In some embodiments, the disease or condition is a cancer. In some embodiments, the disease or condition is an infectious disease (e.g., HIV infection or HCV infection).

Any suitable cancer may be treated with the IL-2 prodrugs provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the IL-2 prodrugs provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is triple-negative breast cancer (TNBC). In some embodiments, the disease is lung cancer. In some embodiments, the disease is non-small cell lung cancer (NSCLC). In some embodiments, the disease is head and neck cancer. In some embodiments, the disease is renal cell carcinoma. In some embodiments, the disease is brain carcinoma. In some embodiments, the disease is endometrial cancer.

### Combination Products

Further provided are combination products comprising an IL-2 prodrug or composition as disclosed herein. In particular embodiments, the IL-2 prodrug is contained within a medical delivery device. Medical delivery device has the definition set forth in Section 201 (h) and includes but not limited to syringes, autoinjectors, medical pens, pumps, and the like.

In particular embodiments, the combination product comprises a therapeutic agent and an IL-2 prodrug that is physically, chemically, or otherwise combined or mixed and produced as a single entity.

The combination product further includes embodiments in which the IL-2 prodrug is packaged separately and is intended for use only with an approved individually specified therapeutic agent or device where both are required to achieve the intended use, indication, or effect and where upon approval of the IL-2 prodrug the labeling of the approved product would need to be changed, e.g., to reflect a change in intended use, dosage form, strength, route of administration, or significant change in dose.

The combination product further includes embodiments in which the IL-2 prodrug is packaged separately and which according to its proposed labeling is for use only with another individually specified investigational therapeutic agent or device where both are required to achieve the intended use, indication, or effect.

In particular embodiments, the therapeutic agent is a checkpoint inhibitor such as a PD-1 blocking agent is an anti-PD-1 antibody or anti-PD-L1 antibody. Exemplary anti-PD-1 antibodies that may be used in the combination therapy of the present invention include any antibody that binds PD-1 and inhibits PD-1 from binding PD-L1. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable carrier or salt.

Pembrolizumab, also known as KEYTRUDA, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013). Nivolumab, also known as OPDIVO, MDX-1106-04, ONO-4538, or BMS-936558, is a fully human IgG4 anti-PD-1 antibody described in WO2006/121168 and U.S. Pat. No. 8,008,449. Cemiplimab-rwlc, also known as cemiplimab, LIBTAYO or REGN2810, is a recombinant human IgG4 monoclonal antibody that is described in WO2015112800 and U.S. Pat. No. 9,987,500.

In another embodiment, the therapeutic agent is an anti-CTLA-4 antibody. In a further embodiment, anti-CTLA-4 antibody is ipilimumab, which is disclosed in U.S. Pat. No. 6,984,720 and WHO Drug Information 19 (4): 61 (2005). In another embodiment, the anti-CTLA-4 antibody is tremelimumab, also known as CP-675,206, which is an IgG2 monoclonal antibody which is described in U.S. Patent Application Publication No. 2012/263677, or PCT International Application Publication Nos. WO 2012/122444 or 2007/113648 A2.

In particular embodiments, the therapeutic agent is a chemotherapy agent. Exemplary chemotherapy agents include but are not limited to
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, axitinib, bortezomib, cabotanzinib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
(xiii) vinca alkaloids and derivatives, including but not limited to, vinblastine, vincristine, vindesine, and vinorelbine.

### Combination Therapy

The present invention provides combination therapies for the treatment of a human or animal individual comprising administering an IL-2 prodrug of the present invention and a second therapeutic agent consecutively or concurrently to the individual. In one embodiment, the IL-2 prodrug is administered to an individual at a time prior to a time the individual is administered the therapeutic agent. In another embodiment, the therapeutic agent is administered to an individual at a time before the individual is administered the IL-2 prodrug. The IL-2 prodrug and therapeutic agent may be administered in separate doses and in different formats.

In particular embodiments, the therapeutic agent is a checkpoint inhibitor such as a PD-1 blocking agent. The PD-1 blocking agent may be administered at the same dose, dosing frequency, and treatment duration as that approved for the PD-1 blocking agent in a monotherapy for particular indications. The dose of the IL-2 prodrug may be administered at the same dosing frequency and treatment duration as approved by the United States Food and Drug Administration (U.S. FDA) or at a dosing frequency and treatment duration as for the particular PD-1 blocking agent that is paired with IL-2 prodrug.

In particular embodiments, the PD-1 blocking agent is an anti-PD-1 antibody or anti-PD-L1 antibody. Exemplary anti-PD-1 antibodies that may be used in the combination therapy of the present invention include any antibody that binds PD-1 and inhibits PD-1 from binding PD-L1. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable salt.

The particular dose of the currently marketed anti-PD-1 antibodies vary between the antibodies, thus in particular embodiments of the combination therapy of the present invention, the dose, dosing frequency, and/or treatment duration may be at least the same as that approved by the U.S. FDA for the particular anti-PD-1 antibody for particular indications. For example, pembrolizumab is approved for a dose of 200 mg every three weeks as needed (pediatric individuals (two years up to 18 years) at 2 mg/kg up to 200 mg every three weeks as needed); nivolumab is approved at a dose of 3 mg/kg every 2 weeks; cemiplimab-rwlc is approved for a dose of 350 mg every three weeks as needed; atezolizumab is approved for a dose of 1200 mg every three weeks as needed; avelumab is approved for a dose of 10 mg/kg or 800 mg every two weeks as needed; and durvalumab is approved for a dose of 10 mg/kg every two weeks as needed.

In particular embodiments of the combination therapy, the PD-1 blocking agent is an anti-PD-1 antibody or anti-PD-1 antibody fragment, which may be administered at a dose from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 antibody or antigen binding fragment thereof is 150 mg, 175 mg, 200 mg, 225 mg, 240 mg, or 250 mg. In further embodiments, the anti-PD-1 antibody or anti-PD-1 antibody fragment may be administered at a frequency of every three weeks as needed. In another embodiment of the combination therapy of the present invention, the anti-PD-1 antibody or anti-PD-1 antibody fragment may be administered at dose greater than 250 mg, for example, a dose of about 400 mg at a frequency of every six weeks as needed.

In particular embodiments of the combination therapy, the PD-1 blocking agent is an anti-PD-1 antibody or anti-PD-1 antibody fragment, which may be administered at a dose from about 10 mg/kg to about 1200 mg. In further embodiments, the PD-1 blocking agent fragment may be administered at a frequency of every two to three weeks as needed.

While the PD-1 blocking agent may be administered at least at the doses, dosing frequencies, and treatment durations approved for the currently marketed PD-1 blocking agents in a monotherapy, the actual doses, dosing frequencies, and treatment durations for any particular combination of the present invention may differ from those that are approved for the PD-1 blocking agent monotherapies. Thus, in particular embodiments of the combination therapy of the present invention, the dose, dosing frequency, and treatment duration of any particular PD-1 blocking agent in the combination therapy will be determined from clinical trials conducted for the combination therapy.

In a particular embodiment of the combination therapy, the PD-1 blocking agent is nivolumab or an effector-silent variant of nivolumab, which is administered to an individual intravenously at a dose of 3 mg/kg over 30 to 60 minutes every two-three weeks as needed and wherein each dose of the IL-2 prodrug is administered intravenously following the administration of the PD-1 blocking agent for the same treatment duration as the PD-1 blocking agent or for duration less than or more than the PD-1 blocking agent duration. In a particular embodiment, the nivolumab or effector-silent variant of nivolumab is administered intravenously to an individual at an initial dose of 3 mg/kg intravenously over 30 minutes followed by administration of the IL-2 prodrug intravenously over 30 minutes on the same day, every three weeks for four doses, then nivolumab is administered intravenously at a fixed dose of 240 mg every two weeks over 30 minutes or 480 mg every four weeks over 30 minutes.

In a particular embodiments, the PD-1 blocking agent is pembrolizumab or effector-silent variant of pembrolizumab, which is administered to an adult individual intravenously at a dose of 200 mg over 30 minutes every three weeks as needed or to a pediatric individual intravenously at a dose of 2 mg/kg up to a maximum of about 200 mg over 30 minutes every three weeks wherein each treatment is followed by a dose of the IL-2 prodrug wherein each dose of the IL-2 prodrug is administered intravenously following administration of the PD-1 blocking agent for the same treatment duration as the PD-1 blocking agent or for duration less than or more than the PD-1 blocking agent duration.

In a particular embodiments, the PD-1 blocking agent is pembrolizumab or effector-silent variant of pembrolizumab, which is administered to an adult individual intravenously at a dose of 400 mg over 30 minutes every six weeks as needed wherein each treatment is followed by a dose of the IL-2 prodrug wherein each dose of the IL-2 prodrug is administered intravenously following the administration of the PD-1 blocking agent for the same treatment duration as the PD-1 blocking agent or for duration less than or more than the PD-1 blocking agent duration.

In a particular embodiment of the combination therapy, the PD-1 blocking agent is cemiplimab-rwlc or an effector-silent variant of cemiplimab-rwlc, which is administered to an individual intravenously at a dose of 350 mg over 30 minutes every three weeks as needed and wherein each dose of the IL-2 prodrug is administered intravenously following the administration of the PD-1 blocking agent for the same treatment duration as the PD-1 blocking agent or for duration less than or more than the PD-1 blocking agent duration. In a particular embodiment, the cemiplimab-rwlc or effector-silent variant of cemiplimab-rwlc is administered intravenously to an individual at an initial dose of 350 mg over 30 minutes followed by administration of the IL-2 prodrug over 30 minutes on the same day every three weeks as needed.

In a particular embodiment of the combination therapy, the PD-1 blocking agent is atezolizumab or an effector-silent variant of atezolizumab, which is administered to an individual intravenously at a dose of 1200 mg over 60 minutes every three weeks as needed and wherein each dose of the IL-2 prodrug is administered intravenously following the administration of the PD-1 blocking agent for the same treatment duration as the PD-1 blocking agent or for duration less than or more than the PD-1 blocking agent duration. In a particular embodiment, the atezolizumab or effector-silent variant of atezolizumab is administered intravenously to an individual at an initial dose of 1200 mg over 60 minutes followed by administration of the IL-2 prodrug over 30 minutes on the same day every three weeks as needed.

In a particular embodiment of the combination therapy, the PD-1 blocking agent is avelumab or an effector-silent variant of avelumab, which is administered to an individual intravenously at a dose of 10 mg/kg or 800 mg over 60 minutes every two weeks as needed and wherein each dose of the IL-2 prodrug is administered intravenously following the administration of the PD-1 blocking agent for the same treatment duration as the PD-1 blocking agent or for duration less than or more than the PD-1 blocking agent duration. In a particular embodiment, the avelumab or effector-silent variant of avelumab is administered intravenously to an individual at an initial dose of 10 mg/kg or 800 mg over 60 minutes followed by administration of the IL-2 prodrug over 30 minutes on the same day every two weeks as needed.

In a particular embodiment of the combination therapy, the PD-1 blocking agent is durvalumab or an effector-silent variant of durvalumab, which is administered to an individual intravenously at a dose of 10 mg/kg over 60 minutes every two weeks as needed and wherein each dose of the IL-2 prodrug is administered intravenously following the administration of the PD-1 blocking agent for the same treatment duration as the PD-1 blocking agent or for duration less than or more than the PD-1 blocking agent duration. In a particular embodiment, the durvalumab or effector-silent variant of durvalumab is administered intravenously to an individual at an initial dose of 10 mg/kg over 60 minutes followed by administration of the IL-2 prodrug over 30 minutes on the same day every two weeks as needed.

While the currently approved PD-1 blocking agents are provided in formulations at a concentration that permits intravenous administration to an individual over a 30 to 60 minute time frame, the combination therapies of the present invention contemplate embodiments in which the IL-2 prodrug and/or the PD-1 blocking agent are each provided in a formulation at a concentration that permits each to be separately administered to an individual in a single injection. Being able to provide at least one of the two blocking agents in a single injection would significantly reduce the time for administering both blocking agents to the individual.

In a further embodiment, the present invention provides a combination therapy in which the IL-2 prodrug and the PD-1 blocking agent are co-administered at the same time. Co-administration may be accomplished by providing the IL-2 prodrug and PD-1 blocking agents in separate formulations and simultaneously providing each formulation to the individual, either by separate IVs or mixing prior to administering the mixture by IV to the individual by IV, or by separate injection of each formulation into the individual. Co-administration may also be accomplished by providing the IL-2 prodrug and PD-1 blocking agents in a single formulation that is then administered to the individual in a single IV or in a single injection.

The combination therapy of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

For example, pembrolizumab is currently approved by the U.S. Food and Drug Administration (U.S. FDA) for a combination therapy for (i) treating non-small cell lung cancer (NSCLC) comprising pembrolizumab with pemetrexed and platinum chemotherapy or carboplatin and either paclitaxel or nab-paclitaxel; and (ii) treating head and neck squamous cell cancer (HNSCC) comprising pembrolizumab and platinum-containing chemotherapy, and atezolizumab is currently approved for a combination therapy for treating NSCLC comprising bevacizumab (anti-VEGF-A antibody marketed under the tradename AVASTIN), paclitaxel, and carboplatin.

Thus, the present invention contemplates embodiments of the combination therapy of the present invention that further includes a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy further in combination with a chemotherapy step may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy with a chemotherapy step may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Combination Therapy Treatments

The combination therapy of the present invention may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy of the present invention may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

The currently marketed PD-1 blocking agents are approved by the U.S. FDA to treat at least one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma. Thus, the combination therapy of the present invention may be used to treat at least one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Kits

In some embodiments, an IL-2 prodrug provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the IL-2 prodrug. In some embodiments, the IL-2 prodrug is provided in the form of a pharmaceutical composition. In some embodiments, the kit further includes a therapeutic agent other than the IL-2 prodrug. In a further embodiment, the kit comprises a combination product comprising the IL-2 prodrug contained within a medical delivery device.

The following examples are intended to promote a further understanding of the present invention.

### General Methods

Standard methods in molecular biology are described in Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; and Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001), Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, and glycosylation of proteins are described (See, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17 and Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies have been described (See Coligan, et al. (2001) Current Protocols in Immunology, Vol. 1, John Wiley and Sons, Inc., New York and Harlow and Lane (1999) Using Antibodies, Cold Spring Harbor Laboratory Press, Cold Spring Harbor, NY). Standard techniques for characterizing ligand/receptor interactions are available (see, e.g., Coligan, et al. (2001) Current Protocols in Immunology, Vol. 4, John Wiley, Inc., New York).

### Example 1

IL-2 Sites for Para-Azidomethylphenylalanine (pAMF) Incorporation and PEG Conjugation

Individual IL-2 variants were designed to incorporate nonnatural pAMF residues in place of specific residues using Sutro's XpressCF+® cell-free expression platform (Yin et al Sci Rep 2017, 7, 1, 3026). Sites were chosen for pAMF incorporation to enable the conjugation of polyethylene glycol (PEG) moieties via copper-catalyzed azide-alkyne cycloaddition (CuAAC) or a copper-free conjugation method, e.g., strain-promoted azide-alkyne cycloaddition (SPAAC) through dibenzocyclooctyne (DBCO or DIBO).

The co-crystal structure of IL-2 bound to IL-2Rα, IL-2Rγ_{c}, and IL-2Rβ (Stauber et al., 2006, Proc Natl Acad Sci USA 103:2793; pdb code 2ERJ) was analyzed using PyMOL to identify which residues have side-chains that point to the solvent or to the IL-2Rα interface. Such residues were chosen for pAMF incorporation to enable conjugation to PEG. In particular, 12 residues near the N-terminus for pAMF incorporation were chosen that have side-chains pointing to the solvent. Conjugation of PEG to any of these sites were selected to increase the half-life of IL-2, lower the dose requirements, and/or increase the overall exposure of the molecule over time. Incorporation of pAMF and/or conjugation of PEG at these sites were also selected to impact binding affinities for IL-2Rα, IL-2Rγ_{c}, and IL-2Rβ, which can be used to optimize the therapeutic properties as described below. Various IL-2 variants were made using standard mutagenesis or gene synthesis techniques and the positions for incorporating pAMF are shown in Table 1. All variants were constructed from IL-2 variant comprising the aldesleukin amino acid sequence (DesA1_IL-2_C124S, referred to as Ald; SEQ ID NO: 3). All amino acid position numbers recited in the examples and shown in the tables therein are in reference to the amino acid sequence set forth in SEQ ID NO: 2 or 3.

TABLE 1
Il-2 variants

| | | | | PEG- | | | Cell- | | | |
|---|---|---|---|---|---|---|---|---|---|---|
| | pAMF | Amino acid | Parent | linker | His | | based | | | |
| SP# | site(s) | Mutation^{†} | SP# | SC# | Tag | SPR | function | PK | efficacy | PD |
| 10295 | F41 | | 10098 | 578 | | Y | Y | Y | Y | |
| 10300 | F41K34 | | 10286 | 578 | | Y | Y | Y | Y | |
| 10283 | F41Y30K34 | | 10280 | 578 | | Y | Y | Y | Y | |
| 10302 | F41Y30K34 | | 10280 | 579 | | Y | Y | Y | Y | |
| 10504 | S4Y30K34 | | 10480 | 579 | | Y | Y | Y | Y | |
| 10505 | S4Y30K34 | R37A/F41K | 10481 | 579 | | Y | Y | Y | Y | |
| 10721 | S4K34Q73 | | 10681 | 579 | Y | Y | Y | | | |
| 10722 | S4K34F77 | | 10682 | 579 | Y | Y | Y | | | |
| 10723 | S4K34V114 | | 10683 | 579 | Y | Y | Y | | | |
| 10724 | S4Y30K34 | F41K | 10684 | 579 | Y | Y | Y | | | |
| 10725 | S4Y30K34R80 | | 10685 | 579 | Y | Y | Y | | | |
| 10726 | S4Y30K34Q73 | | 10686 | 579 | Y | Y | Y | | | |
| 10727 | S4Y30K34V114 | | 10687 | 579 | Y | Y | Y | | | |
| 10728 | S4Y30K34R80 | F41K | 10688 | 579 | Y | Y | Y | | | |
| 10782 | S4K34V114 | | 10774 | 579 | | Y | Y | Y | | |
| 10784 | S4Y30K34Q73 | | 10776 | 579 | | Y | Y | Y | Y | Y |
| 10785 | S4Y30K34V114 | | 10777 | 579 | | Y | Y | Y | | |
| 10922 | S4Y30K34Q73 | | 10776 | 3361 | | | | Y | Y | |
| 10923 | S4Y30K34Q73 | | 10776 | 3362 | | | | Y | Y | |
| 11475 | S4Y30K34Q73 | | 10776 | 3363 | | | | | | |
| 11476 | S4Y30K34Q73 | | 10776 | 3374 | | | | | | |

^{†}F41K and R37A/F41K mutations reduce or abrogate, respectively, affinity to IL2Rα and have been described in WO2021216478.
Y = Yes, procedure done
SPR-surface plasmon resonance assay; commonly referred to by the tradename Biacore ™ (see Cytiva)

Aldesleukin (DesA1_IL-2_C124S, referred to as Ald; SEQ ID NO: 3) was modified to have a carboxyl-terminus HIS6-tag (SEQ ID NO: 44) (Ald-6HIS) linked via a Gly-Gly-Ser (GGS) linker and to have pAMF incorporated at the indicated sites shown in Table 2. These variants were expressed in XpressCF+® in an overnight reaction in the presence of ¹⁴C-Leucine. The expressability of the IL-2 variants was estimated by ¹⁴C-incorporation (total yield), and the amount remaining in solution (soluble yield) was further measured following centrifugation at 14,000×g for 10 minutes. The measured yields are described in Table 2.

TABLE 2
Expression and solubility of Ald-6HIS with
pAMF incorporation at the indicated site

| | Position of pAMF | Total Yield | Soluble Yield |
|---|---|---|---|
| SP# | incorporation in Ald-6HIS | (μg/mL) | (μg/mL) |
| Ald-6His | None | 692 | 463 |
| SP9954 | S4 | 579 | 308 |
| SP9957 | Y30 | 620 | 429 |
| SP9833 | K34 | 593 | 485 |
| SP9960 | F41 | 655 | 419 |
| SP9965 | Q73 | 595 | 407 |
| SP9942 | F77 | 543 | 338 |
| SP9968 | R80 | 523 | 342 |
| SP9970 | V114 | 612 | 371 |

The pAMF residues were chosen to allow conjugation of a non-degradable, non-cleavable PEG. In this design, the conjugated PEG was intended to allow binding to all three IL-2R receptors and increase half-life. As in the above approach, it is desirable to have selective affinity for IL-2Rβ and IL-2Rγ_{c} over affinity for IL-2Rα to increase the anti-tumor response of T-cells and NK-cells with minimal activation of immunosuppressive T_{reg} cells. In this design, however, the resulting PEG-IL-2 conjugates were active upon initial dosing. Conversely, some binding to IL-2Rα may be beneficial to reduce systemic toxicity through stimulation of T_{reg} cells or other IL-2Rα-expressing cell populations. To this end, we sought to identify sites for pAMF incorporation that would allow conjugation of a labile PEG that would mask IL-2 from engagement with IL-2 receptor components. As the PEG is released, the half-life extended IL-2 would be slowly demasked over time, allowing a timed-release of active IL-2. Overall, the longer half-life and slow release of active IL-2 may have a more preferable dosing regimen and enhanced exposure profile over standard IL-2 based therapies, e.g., aldesleukin, with the potential to lower Cₘₐₓ-driven toxicities and widen therapeutic index.

The solubility of some variants appeared to be impacted by protein folding, stability, or aggregation-propensity of IL-2 with pAMF substitution at some sites. The top three IL-2 variants with the highest soluble yields were selected for further evaluation and were accordingly expressed and purified by IMAC resin purification followed by secondary purification with Capto® Q resin. The IL-2 variants were then conjugated to a branched 20 kDa PEG (DBCO 2×10 kDa PEG) having the formula

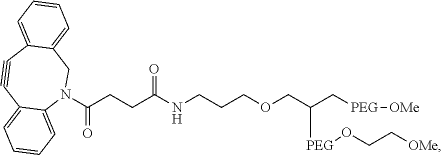

wherein the average MW of each PEG is 10 kDa. The unconjugated and 20 kDa PEG₂-conjugated variants were also evaluated for thermostability (Table 2), IL-2Rα and IL-2Rβ binding (Table 3) and CTLL-2 STAT5 reporter assay (Table 4).

### Example 2

### Differential Scanning Fluorimetry (DSF)

The thermostability measurements provide one measure of the structural impact of pAMF incorporation at the indicated site. A protein thermal shift assay was carried out by mixing the protein to be assayed with an environmentally sensitive dye (SYPRO Orange, Life Technologies Cat #S-6650) in a phosphate buffered solution (PBS), and monitoring the fluorescence of the mixture in real time as it underwent controlled thermal denaturation. Protein solutions were normalized to the same protein concentration (either 0.25, 0.5, or 1 mg/mL) and mixed at a 1-1 volumetric ratio with a 1-500 PBS-diluted solution of SYPRO Orange (SYPRO Orange stock dye is 5000× in DMSO). 10 μL aliquots of the protein-dye mixture were dispensed in quadruplicate in a 384-well microplate (Bio-Rad Cat #MSP-3852, plates pre-heated for 30 minutes at 95° C.), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (about 1.5° C. per minute), allowing three seconds of equilibration at each temperature before taking a fluorescence measurement. At the end of the experiment, the transition melting temperature was determined using the Bio-Rad CFX manager software.

The thermostability of the pAMF-incorporated ald-6HIS variants to understand if pAMF incorporation was well-tolerated at the indicated sites (Table 3). For example, pAMF incorporation at a given site may destabilize IL-2 compared to aldesleukin-HIS, but pAMF incorporation at another site may increase the stability and therefore melting temperature (Tm). Conjugation of PEG may further stabilize or destabilize the molecule by increasing the structural constraints and mobility of IL-2. These attributes are important as they may contribute to biophysical properties that impact pharmacokinetics and drug exposure. Protein thermostability can also impact stability of drug product during production or a drug's shelf-life.

TABLE 3
Thermostability of Ald-6HIS with pAMF
incorporation at the indicated site

| | | Differential Scanning Fluorimetry, | |
|---|---|---|---|
| | | Tm (° C.) | |
| | | Uncon- | DBCO 2 × 10 kDa |
| SP# | Construct | jugated | PEG (PEG2) |
| Ald-6His | Ald-6HIS | 52.8 | NA |
| SP9954 | Ald-6HIS_S4pAMF | 50.4 | 58.8 |
| SP9957 | Ald-6HIS_Y30pAMF | 51.7 | 58.6 |
| SP9833 | Ald-6HIS_K34pAMF | 52.9 | 59.2 |
| SP9960 | Ald-6HIS_F41pAMF | 50.9 | 60.9 |
| SP9965 | Ald-6HIS_Q73pAMF | 53.4 | 58.7 |
| SP9942 | Ald-6HIS_F77pAMF | 52.6 | 57.2 |
| SP9968 | Ald-6HIS_R80pAMF | 50.3 | 51.5 |
| SP9970 | Ald-6HIS_V114pAMF | 53.2 | 58.6 |

NA = not applicable

### Example 3

Label-Free Kinetic Analysis with Surface Plasmon Resonance (SPR)

This example describes methods to identify aldesleukin variants that are pegylated at sites allowing for (1) limited/no impact on IL-2R binding, (2) reduction in IL-2Rα binding while maintaining similar IL-2Rβ, or (3) reduction in IL-2Rβγ_{c} binding compared to recombinant human IL-2 (rhIL-2). This example also provides methods to assess whether binding properties are altered only by PEG-conjugation. As such, a series of label-free assays are used to determine relative binding affinities between the aldesleukin variants and various components of the IL-2R complex.

Anti-Fc polyclonal antibodies were immobilized onto a CM5 chip (GE Life Sciences) using amine coupling chemistry (from Amine Coupling Kit, GE Life Sciences). The immobilization steps were carried out at a flow rate of 25 μL/minute in 1×HBS-EP+ buffer (GE Life Sciences; 10× Stock diluted before use). The sensor surfaces were activated for 7 minutes with a mixture of NHS (0.05 M) and EDC (0.2 M). The anti-Fc antibodies were injected over all four flow cells at a concentration of 25 μg/mL in 10 mM sodium acetate, pH 4.5, for seven minutes. Ethanolamine (1 M, pH 8.5) was injected for seven minutes to block any remaining activated groups. An average of 12,000 response units (RU) of capture antibody was immobilized on each flow cell.

Kinetic binding experiments were performed at 25° C. using 1×HBS-EP+ with 0.05% BSA buffer. IL-2Rα-Fc or IL-2Rβ-Fc (Acro Biosystems, catalog #ILA-H5251, ILB-H5253) was injected over the anti-Fc surface at concentrations of 3-10 μg/mL for 12 seconds at a flow rate of 10 μL/minute on flow cells 2, 3 and 4, followed by a buffer wash for 30 seconds at the same flow rate. Kinetic characterization of conjugated or unconjugated aldesleukin or variants was carried out in a range of concentrations from 1 nM-10 μM and one injection of no antigen. After capturing ligand (IL-2Rα-Fc or IL-2Rβ-Fc) on the anti-Fc surface, the analyte (IL-2 variant) was bound for 60 seconds, followed by a 180 second dissociation phase at a flow rate of 50 μL/min. Between each ligand capture and analyte binding cycle, regeneration was carried out using two injections of 10 mM Glycine pH 1.5 for 30 seconds at 30 μL/minute, followed by a 30 second buffer wash step. The data were fit with the Biacore™ T200 Evaluation software and are shown in Table 4.

The kinetic affinity of Ald-6HIS variants to IL-2Rα-Fc and IL-2Rβ-Fc was measured to understand if pAMF incorporation and subsequent conjugation impacted the ability of the IL-2 variants to engage with IL-2 receptors. For instance, in some cases it may be preferred for pAMF incorporation (with or without PEG conjugation) to interfere with IL-2Rα engagement by reducing or abolishing its binding. This may be preferred to create an IL-2 receptor biased interaction to preferentially activate specific immune populations that have varying expression levels of IL-2Rα. Alternatively, some sites of pAMF incorporation and conjugation may result in an attenuated IL-2Rβ binding and signaling in the presence of a PEG, but be rescued once that PEG molecule is released. Finally, for an IL-2 prodrugged concept, some sites of conjugation may be necessary to provide additional half-life extension and may not interfere with binding to either IL-2Rα or IL-2Rβ.

TABLE 4
Kinetic affinity of Ald-6HIS variants (pAMF incorporated, with or without conjugation
to DBCO-2 × 10 kDa PEG) to IL-2Rα-Fc and IL-2Rβ-Fc

| | | SPR IL-2Rα-Fc | | SPR IL-2Rβ-Fc | |
|---|---|---|---|---|---|
| | | binding, (M) | | binding, (M) | |
| | | | DBCO 2 × 10 | | DBCO 2 × 10 |
| | | | kDa PEG | | kDa PEG |
| | | | (PEG₂) | | (PEG₂) |
| SP# | Construct | Unconjugated | conjugated | Unconjugated | conjugated |
| Ald-6His | Ald-6HIS | 8E−09 | NA | 3E−07 | NA |
| SP9954 | Ald-6HIS_S4pAMF | 8E−09 | 1E−07 | 3E−07 | NC |
| SP9957 | Ald-6HIS_Y30pAMF | 9E−09 | 3E−07 | 2E−07 | NC |
| SP9833 | Ald-6HIS_K34pAMF | 7E−09 | 8E−07 | 3E−07 | NC |
| SP9960 | Ald-6HIS_F41pAMF | NC | NC | 2E−07 | NC |
| SP9965 | Ald-6HIS_Q73pAMF | 7E−09 | NC | 8E−08 | NC |
| SP9942 | Ald-6HIS_F77pAMF | 1E−08 | NC | NC | NC |
| SP9968 | Ald-6HIS_R80pAMF | 2E−08 | 1E−07 | 4E−07 | NC |
| SP9970 | Ald-6HIS_V114pAMF | 5E−08 | 5E−07 | 3E−07 | NC |

NA = not applicable; NC = not calculable

### Example 4

### GloResponse® STAT5-Luc2-CTLL-2 Reporter Assay

GloResponse® STAT5-luc2-CTLL-2 (Promega, CD2018B05) cells were purchased from Promega (CS201805) and maintained in complete RPMI-1640 (Corning) with 100 IU Penicillin/100 μg/mL Streptomycin (Corning), 2 mM GlutaMax® (Gibco), 20% heat-inactivated FBS (Sigma), and 10 ng/ml IL-2 (Peprotech). On assay day, cells were starved of IL-2 for at least 4 hours prior to treatment. Then, 25 μL of 0.5×10⁶ cells/mL cells were seeded in complete culture medium in a standard white TC-coated 384-well plate. Cells were treated with 25 μL of serial dilution of aldesleukin or variant samples (1:8 serial dilution of 1 μM starting concentration) and then incubated at 37° C., 5% CO₂ for 24 hours. Then, 30 μL of reconstituted Bio-Glo (Promega) reagent was added and allowed to incubate for 25 minutes at room temperature with shaking. Plates were read on the Envision plate reader (PerkinElmer) and luminescence readings were converted to % relative signal using the 1 μM wild-type IL-2 (aldesleukin) treated cells as controls. Data was fitted with non-linear regression analysis, using log (against) vs. response, variable slope, four-parameter fit equation using GraphPad Prism and the results are shown in Table 5.

TABLE 5
CTLL-2 STAT5 reporter assay

| | | | DBCO 2 × 10 |
|---|---|---|---|
| | | | kDa PEG |
| | | | (PEG₂) |
| | | Unconjugated | conjugated |
| SP# | Construct | EC50 (pM) | EC50 (pM) |
| Ald-6His | Ald-6HIS | 12 | NA |
| SP9954 | Ald-6HIS_S4pAMF | 11 | 209 |
| SP9957 | Ald-6HIS_Y30pAMF | 10 | 302 |
| SP9833 | Ald-6HIS_K34pAMF | 6 | 474 |
| SP9960 | Ald-6HIS_F41pAMF | 9 | 4479 |
| SP9965 | Ald-6HIS_Q73pAMF | 10 | 955 |
| SP9942 | Ald-6HIS_F77pAMF | 11 | 318 |
| SP9968 | Ald-6HIS_R80pAMF | 12 | 303 |
| SP9970 | Ald-6HIS_V114pAMF | 26 | 2772 |

NA = not applicable

### Example 5

### NK-92 and DERL-7 Cell Proliferation Assay

NK-92 (IL2RABG, ATCC, CRL-2407) and DERL-7 (IL2RBG, DSMZ, ACC 524) cells were maintained in complete RPMI-1640 (Corning) with 100 IU Penicillin/100 μg/mL Streptomycin (Corning), 2 mM GlutaMax (Gibco), 20% heat-inactivated FBS (Sigma), and 10 ng/ml IL-2 (Peprotech). On assay day, cells were starved of IL-2 for at least four hours prior to treatment. Then, 25 μL of 0.5×10⁶ cells/mL cells were seeded in complete culture medium in a standard white TC-coated 384-well plate. Cells were treated with 25 μL of serial dilution of aldesleukin or variant samples (1:8 serial dilution of 1 uM starting concentration) and then incubated at 37° C., 5% CO₂ for 24 hours. Then, 30 μL of reconstituted CellTiter-Glo® (Promega) reagent was added and allowed to incubate for 25 minutes at room temperature, with shaking. Plates were read on the Envision plate reader (PerkinElmer) and luminescence readings were converted to % relative signal using the 1 μM aldesleukin treated cells as controls. Data was fitted with non-linear regression analysis, using log (against) vs. response, variable slope, four-parameter fit equation using GraphPad Prism and the results are shown in Table 6.

TABLE 6
Cell Based Proliferation Assays

| | | | | DERL-7 EC50 (pM) | |
|---|---|---|---|---|---|
| | | NK-92 EC50 (pM) | | | DBCO |
| | | | DBCO 2 × 10 | | 2 × 10 kDa |
| | | | kDa PEG | | PEG |
| | | | (PEG₂) | | (PEG₂) |
| SP# | Construct | Unconjugated | conjugated | Unconjugated | conjugated |
| Ald-6His | Ald-6HIS | 7.53 | NA | 51.8 | NA |
| SP9954 | Ald-6HIS_S4pAMF | 9.10 | 55.8 | 43.3 | 327 |
| SP9957 | Ald-6HIS_Y30pAMF | 4.04 | 21.6 | 32.7 | 1362 |
| SP9833 | Ald-6HIS_K34pAMF | 6.10 | 79.0 | 28.8 | 498 |
| SP9960 | Ald-6HIS_F41pAMF | 9.89 | 101.2 | 58.1 | 142 |
| SP9965 | Ald-6HIS_Q73pAMF | 7.08 | 37.3 | 14.2 | 1567 |
| SP9942 | Ald-6HIS_F77pAMF | 5.45 | 60.2 | 19.5 | 6380 |
| SP9968 | Ald-6HIS_V114pAMF | 6.59 | 156.7 | 37.9 | 1043 |

NA = not applicable

### Example 6

Synthesis of mPEG or mPEG₂ Fmoc-DBCO Linkers

### SC547 Structure:

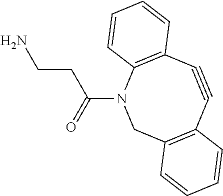 [SMILES: NCCC(=O)N1Cc2ccccc2C#Cc2ccccc21]

SC547 was obtained from Click Chemistry Tools (CAS 1255942-06-3; Cat #A103-25).
SC578 synthesis: SC578 mPEG₂ (2×10 kDa) Fmoc-DBCO linker is synthesized as shown below in Scheme 1.

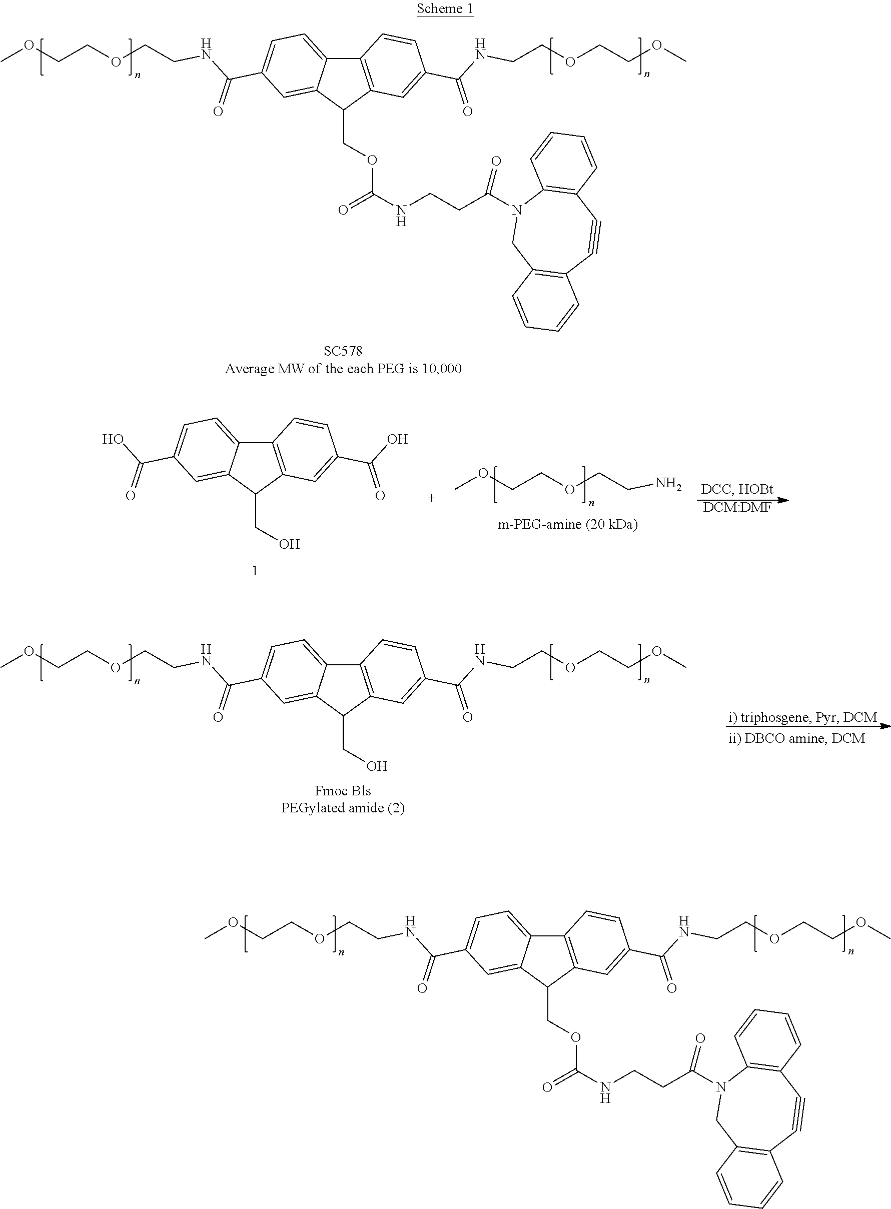

In a 500 mL round bottom flask was added mPEG-NH₂ (10,000) (30 g, 2.3 mmol) in anhydrous toluene (250 mL). The mixture was azeotropically dried under reduced pressure at 45° C. on a rotary evaporator, and dried in vacuum for overnight, and then dissolved in anhydrous 200 mL dichlormethane (DCM). A solution of 9-(hydroxymethyl)-9H-fluorene-2,7-dicarboxylic acid (Compound 1) (0.35 g, 1.24 mmol) and hydroxybenzotriazole (HOBt); 0.67 g, 4.9 mmol) was dissolved in anhydrous 20 mL dimethylformamide (DMF) and added to the mPEG-NH₂ (10,000) solution. Thereafter, DCC (1.02 g, 4.96 mmol) was added. The reaction was stirred at rt for one day under N₂ atmosphere. Reaction progress was monitored by analytical HPLC-ELSD (Column: Jupiter C₄: LC column 250 mm×4.6 mm×5 μm (Vendor: Phenomenex, part #00G167-E0), Mobile Phase: Acetonitrile and Water with 0.1% TFA (90% water to 10% water in 50 minutes, flow rate, 1 mL/minute). Thereafter, HPLC showed completion of the reaction, solvents were removed under reduced pressure, and the crude PEG product was added to isopropanol (600 mL) with gentle heating (35° C.). To this was added 200 mL Methyl tertiary-butyl ether (MTBE) and then the mixture was cooled to 10° C. Solids were filtered and washed with cold IPA (100 mL) and MTBA (50 mL). Crude bis-PEGylated amide 2 as off-white solids were dried under vacuum. The Fmoc bis PEGylated amide 2 was dissolved in DCM (300 mL), added to a Capto® S resin (prewashed with water; 1 L), and stirred overnight. After filtration, the solvent was entirely removed. Matrix-assisted laser desorption/ionization-time of flight (MALDI-TOF), Proton nuclear magnetic resonance (¹H NMR), and analytical evaporative light scattering detector-high-performance liquid chromatography (ELSD-HPLC) confirmed the desired Compound 2 in good purity.

Compound 2 (7 g, 0.35 mmol) (azeotropically dried with 100 mL toluene removed at 50° C. under vacuum prior to use), and anhydrous DCM (50 mL). The clear solution was flushed with argon and then triphosgene (290.8 mg, 0.98 mmol) and pyridine (0.15 mL, 1.89 mmol) were added sequentially. The reaction mixture was stirred at room temperature for two hours under nitrogen. DCM and pyridine were removed under reduced pressure. The chloroformate intermediate 2a was dissolved in 50 mL of DCM, and DBCO amine (339 mg, 1.22 mmol) was added in one portion. The reaction was stirred at room temperature for two hours under N₂ atmosphere. Solvent was removed to dryness, the solids were dissolved in 15 mL of DCM and precipitated via IPA (500 mL). The precipitation was repeated twice. Solids were filtered and dried under vacuum. The resulting Compound SC578 was confirmed by ¹H NMR (CDCl₃), MALDI-TOF, sodium dodecyl sulfate-polyacrylamide gel electrophoresis (SDS-PAGE), and analytical ELSD-HPLC. Purity analysis was checked via the above mentioned analytical HPLC method.

### SC579 Synthesis:

SC579 mPEG (20 kDa) Fmoc-DBCO linker is synthesized as shown below in Scheme 2.

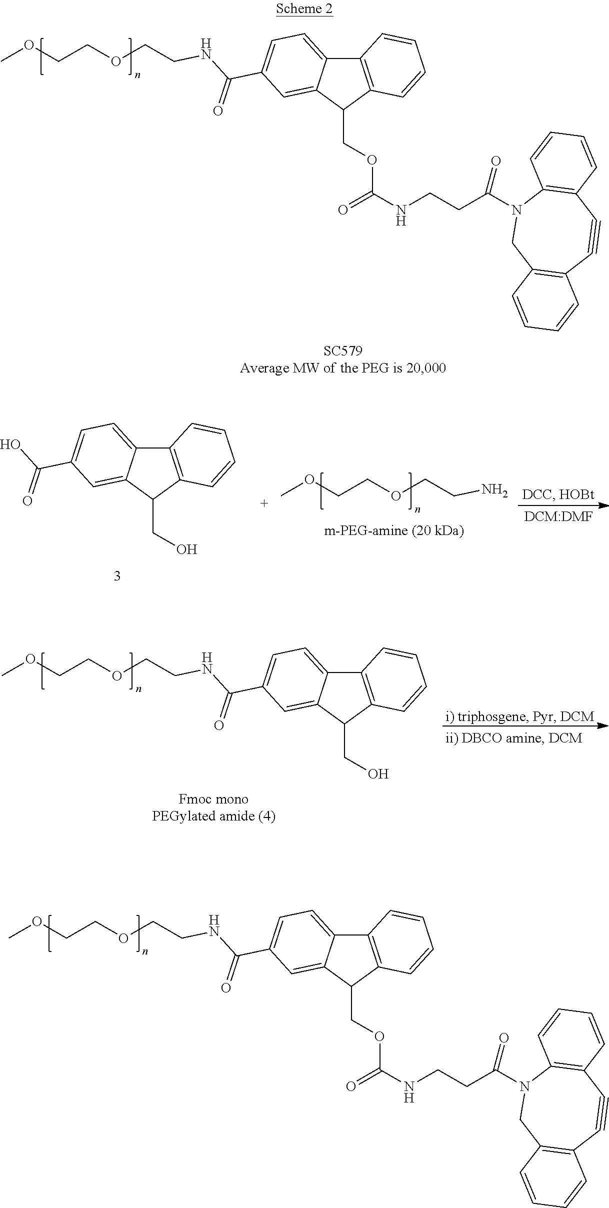

In a 1000 mL flask equipped with a magnetic stir bar, was added mPEG-NH₂ (20,000) (23.98 g, 1.2 mmol) in anhydrous toluene (250 mL). The mixture was azeotropically dried under reduced pressure at 45° C. on a rotary evaporator, lyophilized overnight, and then dissolved in anhydrous DCM (200 mL). A solution of 9-(hydroxymethyl)-9H-fluorene-2-carboxylic acid (Compound 3) (0.72 g, 2.99 mmol) and HOBt (0.61 g, 4.5 mmol) was dissolved in anhydrous DMF (15 mL) and added to the mPEG-NH₂ (20,000) solution. Thereafter, DCC (0.93 g, 4.5 mmol) was added. The reaction was stirred at room temperature for one day under N₂ atmosphere. Reaction progress was monitored by analytical HPLC-ELSD (Column: Jupiter C₄: LC column 250 mm×4.6 mm×5 μm (Vendor: Phenomenex, part #00G167-E0), Mobile Phase: Acetonitrile and Water with 0.1% TFA (90% water to 10% water in 50 min, flow rate, 1 mL/minute). Thereafter, HPLC showed completion of the reaction, solvents were removed under reduced pressure, and the crude PEG product was added to isopropanol (600 mL) with gentle heating (35° C.). To this was added 200 mL MTBE and then the mixture was cooled to 10° C. Solids were filtered and washed with cold IPA (100 mL) and MTBA (50 mL). Crude Fmoc mono-PEGylated amide 4 as off-white solids were dried under vacuum. The PEGylated amide 4 was dissolved in DCM (300 mL), added to a Capto® S resin (prewashed with water; 1 L), and stirred overnight. After filtration, the solvent was entirely removed. MALDI-TOF, ¹H NMR, and HPLC-ELSD confirmed the desired Compound 4 in good purity.

To an oven-dried 250 mL flask equipped with a magnetic stir bar, were added Fmoc mono PEGylated amide Compound 4 (11.4 g, 0.52 mmol) (azeotropically dried with 100 mL toluene removed at 50° C. under vacuum prior to use), and anhydrous DCM (70 mL). The clear solution was flushed with argon and then triphosgene (231.9 mg, 0.78 mmol) and pyridine (0.06 mL, 0.73 mmol) were added sequentially. The reaction mixture was stirred at room temperature for two hours under nitrogen. DCM and pyridine were removed under reduced pressure. The chloroformate intermediate was dissolved in 50 mL of DCM, and DBCO amine (432 mg, 1.56 mmol) was added in one portion. The reaction was stirred at room temperature for two hours under N₂ atmosphere. Solvent was removed to dryness, the solids were dissolved in 15 mL of DCM and precipitated via IPA (500 mL). The precipitation was repeated twice. Solids were filtered and dried under vacuum. The resulting SC579 was confirmed by ¹H NMR (CDCl₃), MALDI-TOF, SDS-PAGE, and analytical ELSD-HPLC. Purity analysis was checked via the above-mentioned analytical method.

### SC3361 Synthesis:

SC3361 mPEG (20 kDa) Fmoc-DBCO linker is synthesized as shown below in Scheme 3.

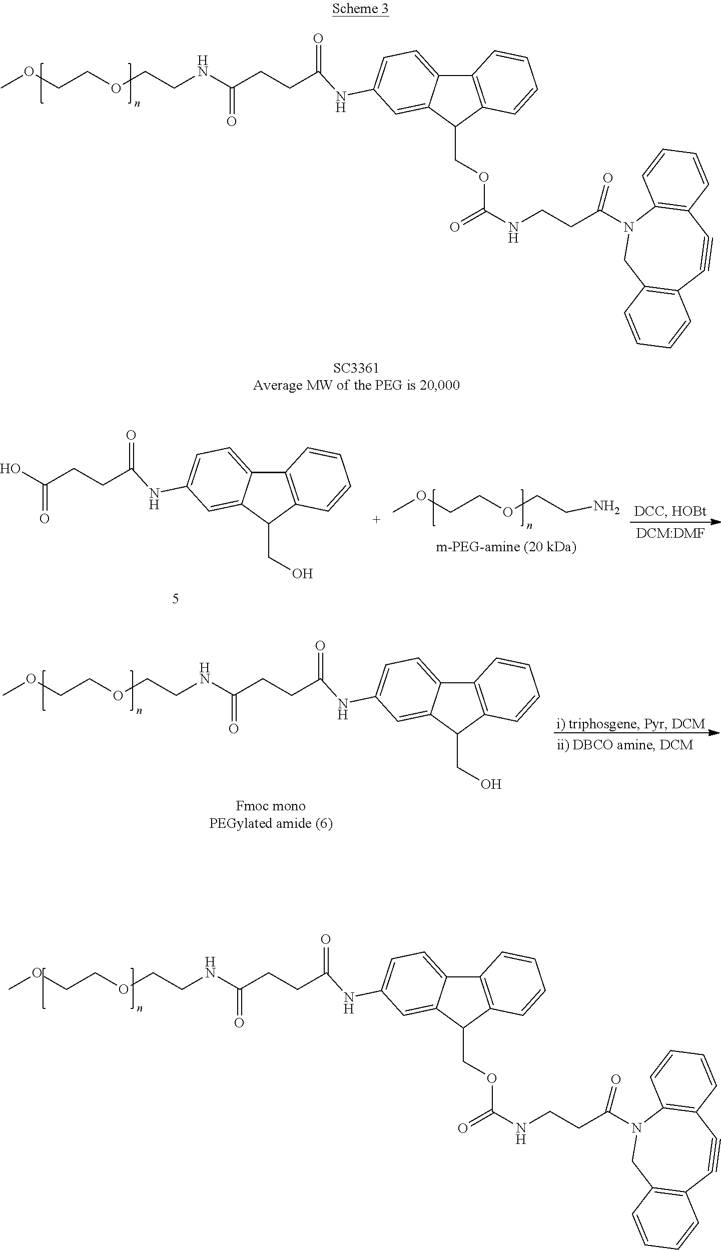

Compound SC3361 was synthesized using the same methods as described above using the 4-((9-(hydroxymethyl)-9H-fluoren-2-yl)amino)-4-oxobutanoic acid as the starting material for PEGylation, and confirmed by ¹H NMR (CDCl₃), MALDI-TOF, SDS-PAGE, and analytical ELSD-HPLC.

### SC3362 Synthesis:

SC3362 mPEG (20 kDa) Fmoc-DBCO linker is synthesized as shown below in Scheme 4.

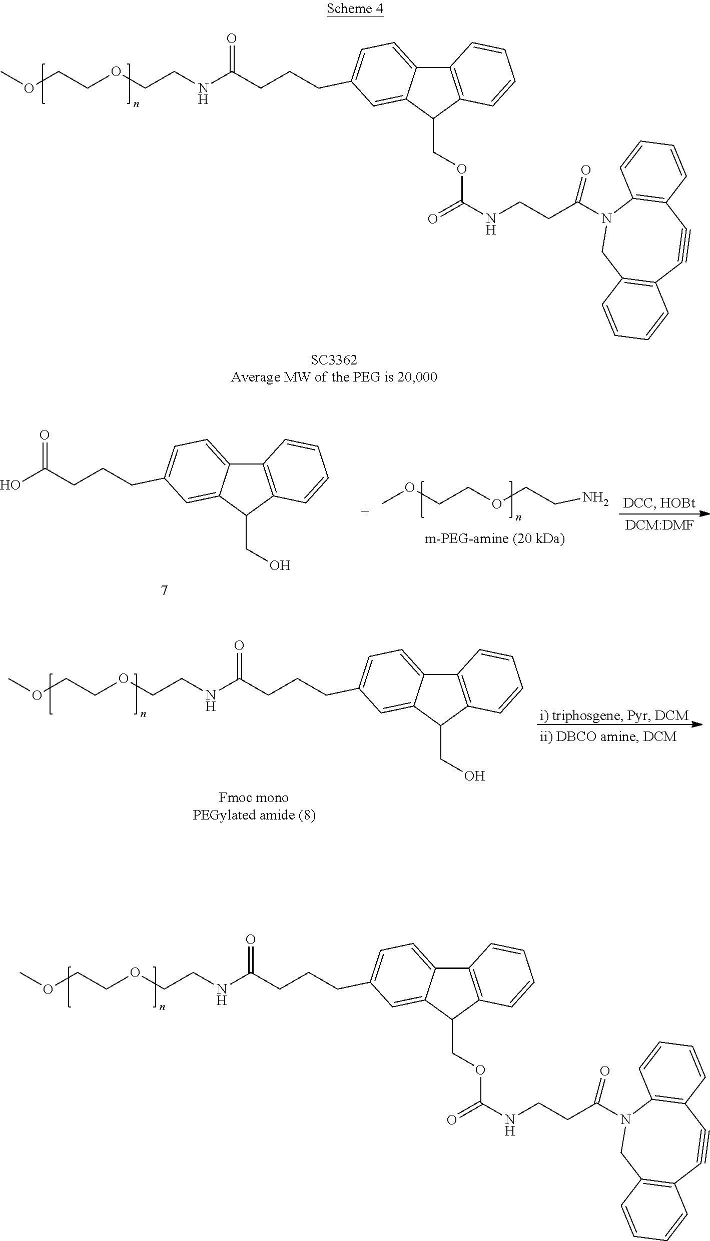

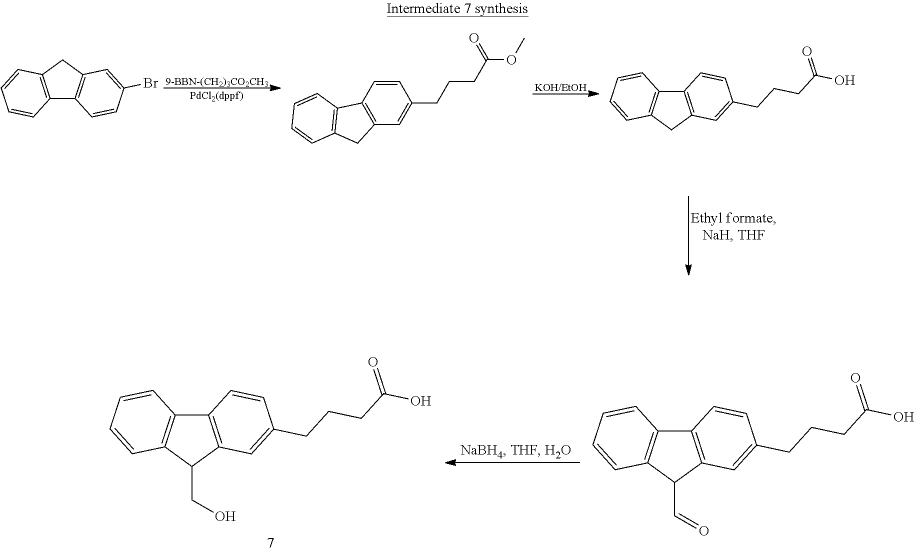 [SMILES: Brc1ccc2c(c1)Cc1ccccc1-2 ; COC(=O)CCCc1ccc2c(c1)Cc1ccccc1-2 ; O=C(O)CCCc1ccc2c(c1)Cc1ccccc1-2 ; O=C(O)CCCc1ccc2c(c1)C(CO)c1ccccc1-2 ; O=CC1c2ccccc2-c2ccc(CCCC(=O)O)cc21]

Compound SC3362 was synthesized using the same methods as described above using the 4-(9-(hydroxymethyl)-9H-fluoren-2-yl) butanoic acid as the starting material for PEGylation, and confirmed by ¹H NMR (CDCl₃), MALDI-TOF, SDS-PAGE, and analytical ELSD-HPLC.

### SC3363 Synthesis:

SC3363 mPEG (20 kDa) Fmoc-DBCO linker is synthesized as shown below in Scheme 5.

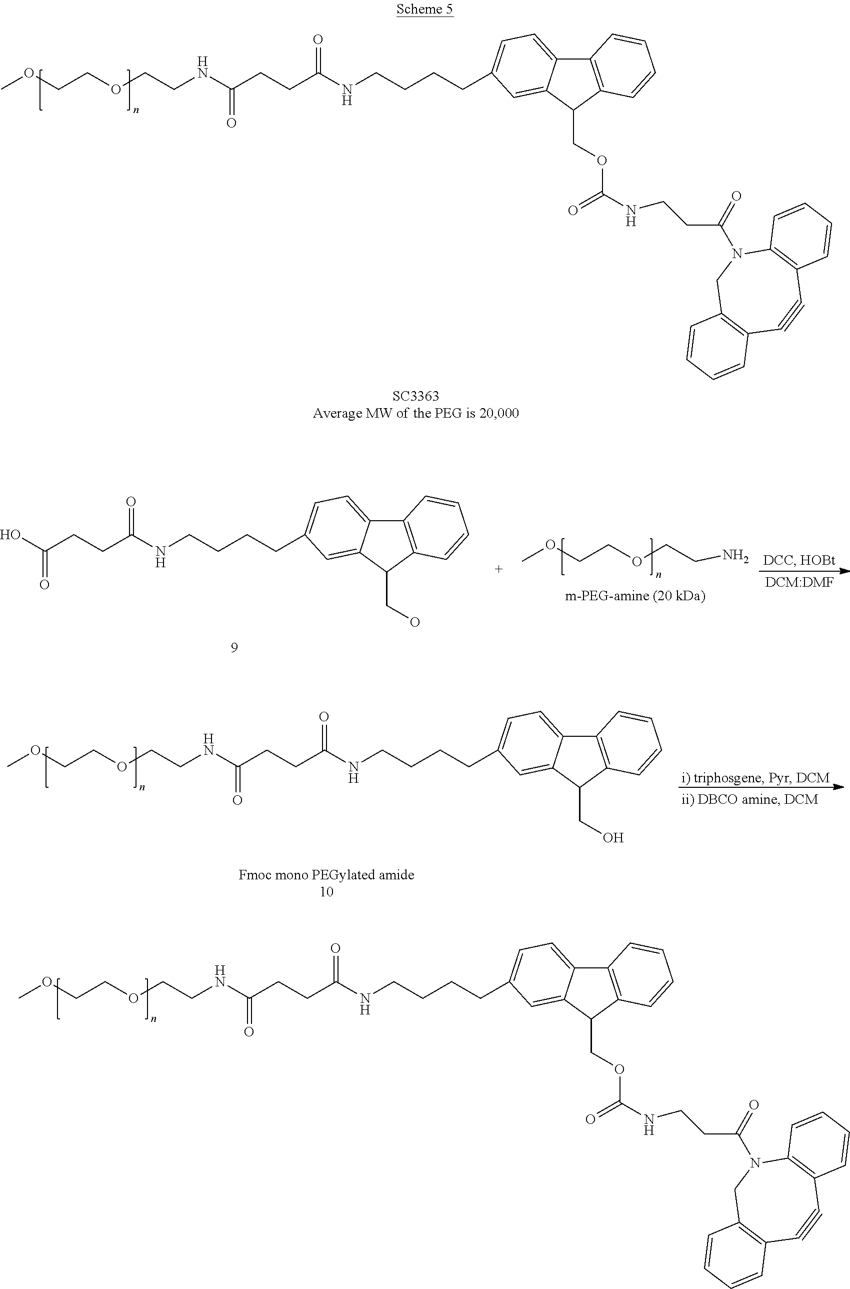

Compound SC3363 was synthesized using the same methods as described above using the 4-((4-(9-(hydroxymethyl)-9H-fluoren-2-yl)butyl)amino)-4-oxobutanoic acid as the starting material for PEGylation, and confirmed by ¹H NMR (CDCl₃), MALDI-TOF, SDS-PAGE, and analytical ELSD-HPLC.

### SC3374 Synthesis:

SC3374 mPEG (20 kDa) Fmoc-DBCO linker is synthesized as shown below in Scheme 6.

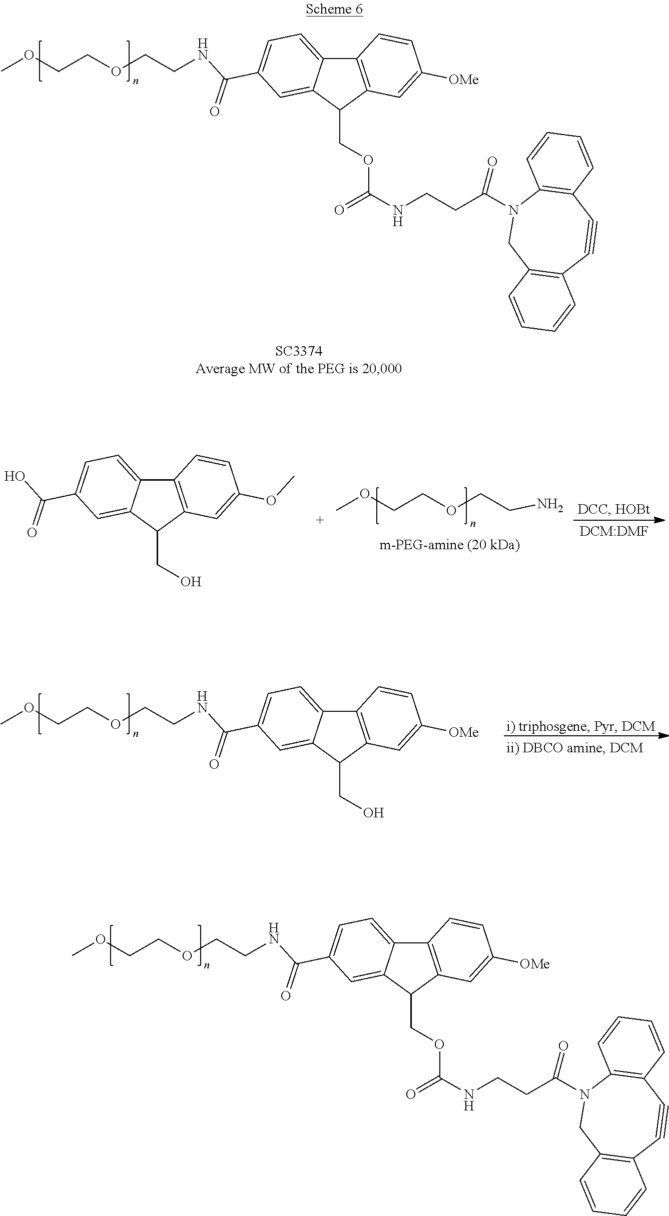

Compound SC3374 was synthesized using the same methods as described above using 9-(hydroxymethyl)-7-methoxy-9H-fluorene-2-carboxylic acid as the starting material for PEGylation, and confirmed by ¹H NMR (CDCl₃), MALDI-TOF, SDS-PAGE, and analytical ELSD-HPLC.

### Example 7

Cell-Free Expression of Recombinant IL-2 (rIL-2) and Variants Containing pAMF

Aldesleukin and variants were expressed in an XpressCF+® reaction. The cell-free extracts were prepared from a mixture of four extracts derived from four engineered strains: (1) an OmpT sensitive RF1 attenuated E. coli strain engineered to overexpress E. coli DsbC and FkpA, (2) a similar RF1 attenuated E. coli strain engineered to produce an orthogonal CUA-encoding tRNA for nonnatural amino acid insertion at an Amber Stop Codon, (3) a similar RF1 attenuated E. coli strain engineered to produce the pAMF-specific amino-acyl tRNA synthetase and (4) a similar RF1 attenuated E. coli strain engineered to produce T7 RNA polymerase. Cell-free extract 1 was treated with 50 μM iodoacetamide for 30 minutes at room temperature (20° C.) and added to a premix containing all other components. The final concentration in the protein synthesis reaction was 30% (v/v) cell extract 1, 1% (v/v) cell extract 2 or 5 μM orthogonal CUA-encoding tRNA, 0.6% (v/v) cell extract 3 or 5 uM engineered pAMF-specific amino-acyl tRNA synthetase, 0.5% (v/v) cell extract 4 or 100 nM T7 RNAP, 2 mM para-azidomethylphenylalanine (pAMF), 2 mM GSSG, 8 mM magnesium glutamate, 10 mM ammonium glutamate, 130 mM potassium glutamate, 35 mM sodium pyruvate, 1.2 mM AMP, 0.86 mM each of GMP, UMP, and CMP, 2 mM amino acids (except 0.5 mM for Tyrosine and Phenylalanine), 4 mM sodium oxalate, 1 mM putrescine, 1.5 mM spermidine, 15 mM potassium phosphate, 2.5-5 μg/mL IL2 or variants DNA. Cell-free reactions were performed at 20-30° C. for 12 hours on a shaker at 650 rpm in 96-well plates at 100 μL scale, in 24-well flower plates at 1 mL scale, in 100×10 mm petri dish at 8 mL scale or in stirred tanks at larger scales.

### Example 8

### Purification of Aldesleukin and Variants

Aldesleukin and variants were constructed with 6×Histidine tag (SEQ ID NO: 44) at N- or C-terminus; cleavable affinity tags, e.g. His SUMO tag at N-terminus; or without a tag. Untagged aldesleukin and variants were purified by standard purification methods. His-tagged aldesleukin (Ald-6HIS) and variants were purified by standard immobilized metal affinity chromatography (IMAC) purification methods. Molecules with cleavable affinity tags were processed by enzymatic digestion followed by standard purification to remove tag and enzyme.

After PEGylation, the reaction consisting of conjugated IL-2 and unreacted PEG was further processed by cation exchange column packed with Capto® SP ImpRes™ resin (Cytiva). Dilution of the IL-2/PEG reaction prior to purification was performed with binding buffer (10 mM acetic acid, pH 4.5) and bound to the Capto® SP ImpRes column with a two-minute residence time during the load. A linear gradient with elution buffer (10 mM acetic acid, 500 mM arginine, pH 4.5) was performed over 30 column volumes (CV) and the target elution fractions were collected and buffer exchanged into 10 mM citric acid, 9% sucrose, pH 4.5 by Amicon® Ultra-15, 10 kD.

The formulated IL-2 prodrug was rendered sterile by passing through a 0.2 micron polyethersulfone (PES) filter prior to storage at −80° C. Analysis of the unconjugated and conjugated IL-2 product pools by SDS-PAGE show a single protein band at the correct molecular weight. Monomer percentage and presence of impurities were checked by HPLC-SEC, performed with Ultimate™ 3000 system and Sepax Zenix®-C SEC-150 (7.8×300 mm) for the unconjugated IL-2 variants, while the conjugated IL-2 prodrug was analyzed with Sepax SRT SEC-500. The unconjugated and conjugated IL-2 prodrug proteins elute as a single peak on the analytical size-exclusion chromatogram with a reported monomer content percentage of >95%.

### Example 9

### Site-Specific PEGylation

Copper-free and Copper-catalyzed conjugation chemistry are utilized to conjugate PEG site specifically to aldesleukin variants containing pAMF.

SPAAC copper-free conjugation: Linear or branched mPEG (10 kDa, 20 kDa, 30 k Da, 40 kDa) was linked to dibenzocyclooctyne (DBCO) or dibenzocyclooctynol (DIBO). A 5 mM stock solution of DBCO/DIBO-mPEG was mixed with 1-50 mg/mL aldesleukin variants incorporated with pAMF at DBCO/DIBO-mPEG to pAMF ratio of 2-50 for 8 hours to 5 days at 22-35° C.

CuAAC conjugation: 5 mM stock solution of linear or branched alkyne-mPEG (10 KDa, 20 KDa, 30 KDa, 40 KDa) was mixed with 1-50 mg/mL aldesleukin variants incorporated with pAMF at alkyne-mPEG to pAMF ratio of 2-50 in phosphate buffer (100 mM sodium phosphate, 150 mM NaCl, pH7.4). Copper reagent was prepared separately by mixing CuSO₄, ligand (triazole based such as tris(3-hydroxypropyltriazolylmethyl)amine, or benzimidazol-based such as tripotassium 5,5′,5″-[2,2′,2″-nitrilotris(methylene)tris(1H-benzimidazole-2,1-diyl)]tripentanoate hydrate), reducing reagent (sodium ascorbate, DTT, or TCEP) and ROS scavenger (methionine, cysteine, or histidine). Aminoguanidine was added when sodium ascorbate was used as reducing reagent. Copper was added at alkyne to copper molar ratio of 1-15, ligand to copper molar ratio is 1-5, reducing reagent to copper molar ratio was 2-10, ascorbate to aminoguanidine molar ratio is 1-5. Copper reagent was then mixed with protein/drug mixture for 8 hours to 5 days at 22-35° C. When anaerobic condition was required, all solutions were purged with inert gas before mixing, and the reaction was kept under inert gas during the reaction.

### Example 10

### PEGylation Density

PEGylation density was determined by gel densitometry. Gel densitometry analysis was used to estimate PEG density. About 1-4 μg of PEGylated IL-2 was loaded on 4-12% Bis-tris SDS-PAGE (NuPAGE™ Invitrogen). The gel ran in 1×NuPAGE™ MES SDS Running Buffer (Invitrogen) with constant voltage at 400 volts for 35 minutes. The gel image was scanned using Gel Doc EZ Imager (Bio-Rad) and exported for densitometry analysis using ImageQuant® TL 7.0 (GE Health). The PEG density of PEGylated aldesleukin variants is shown in Table 7.

TABLE 7
PEG density of PEGylated aldesleukin variants

| | PEGylation | | | His | PEG |
|---|---|---|---|---|---|
| SP# | Site(s) | Mutation^{†} | SC# | Tag | density |
| 10295 | F41 | | 578 | | 0.99 |
| 10300 | F41K34 | | 578 | | 1.80 |
| 10283 | F41Y30K34 | | 578 | | 2.60 |
| 10302 | F41Y30K34 | | 579 | | 2.65 |
| 10504 | S4Y30K34 | | 579 | | 2.80 |
| 10505 | S4Y30K34 | R37A/F41K | 579 | | 2.79 |
| 10721 | S4K34Q73 | | 579 | Y | 3 |
| 10722 | S4K34F77 | | 579 | Y | 3 |
| 10723 | S4K34V114 | | 579 | Y | 3 |
| 10724 | S4Y30K34 | F41K | 579 | Y | 3 |
| 10725 | S4Y30K34R80 | | 579 | Y | 4 |
| 10726 | S4Y30K34Q73 | | 579 | Y | 4 |
| 10727 | S4Y30K34V114 | | 579 | Y | 4 |
| 10728 | S4Y30K34R80 | F41K | 579 | Y | 4 |
| 10782 | S4K34V114 | | 579 | | 3.0 |
| 10784 | S4Y30K34Q73 | | 579 | | 3.8 |
| 10785 | S4Y30K34V114 | | 579 | | 3.7 |
| 10922 | S4Y30K34Q73 | | 3361 | | 3.9 |
| 10923 | S4Y30K34Q73 | | 3362 | | 3.9 |
| 11475 | S4Y30K34Q73 | | 3363 | | 3.9 |
| 11476 | S4Y30K34Q73 | | 3374 | | 3.9 |

^{†}F41K/R37A and F41K mutations abrogate or reduce, respectively, affinity to IL2Rα and have been described in WO2021216478.

### Example 11

In vitro PEG Release Assay

This example compares the release kinetics for the various PEG Fmoc linkers SC578, SC579, SC3361, SC3362, SC3363, and SC3374 conjugated to various aldesleukin variants.

In general, the PEGylated aldesleukin variants (1 mg/mL) were buffer exchanged into 100 mM sodium bicarbonate at pH 9.0 and incubated overnight at 30° C. The released product was analyzed on SDS-PAGE. To determine the release rate of PEG variants, aldesleukin with pAMF incorporation at position S4 was conjugated to the PEG variants. After overnight conjugation, the reaction mixture was buffer exchanged in pH 9.0 100 mM sodium bicarbonate and incubated at 30° C. Samples were collected at different time points and flash frozen until SDS-PAGE analysis. Percentage of released aldesleukin was quantified using densitometry.

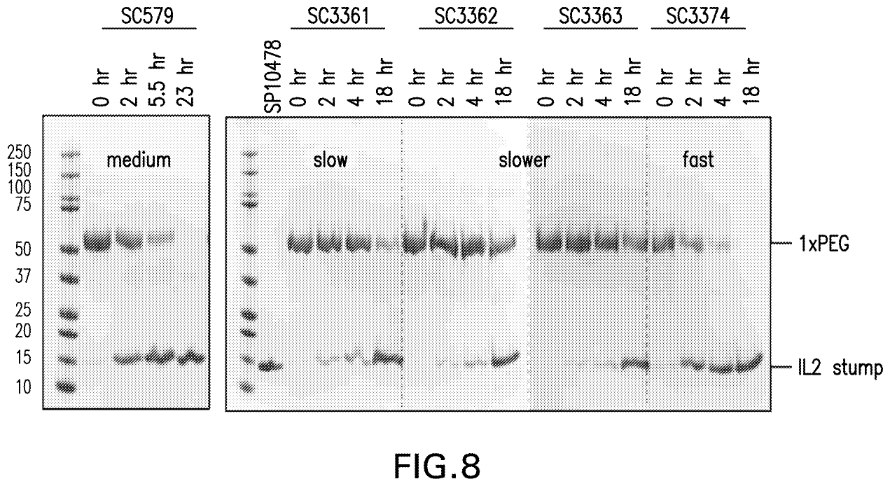

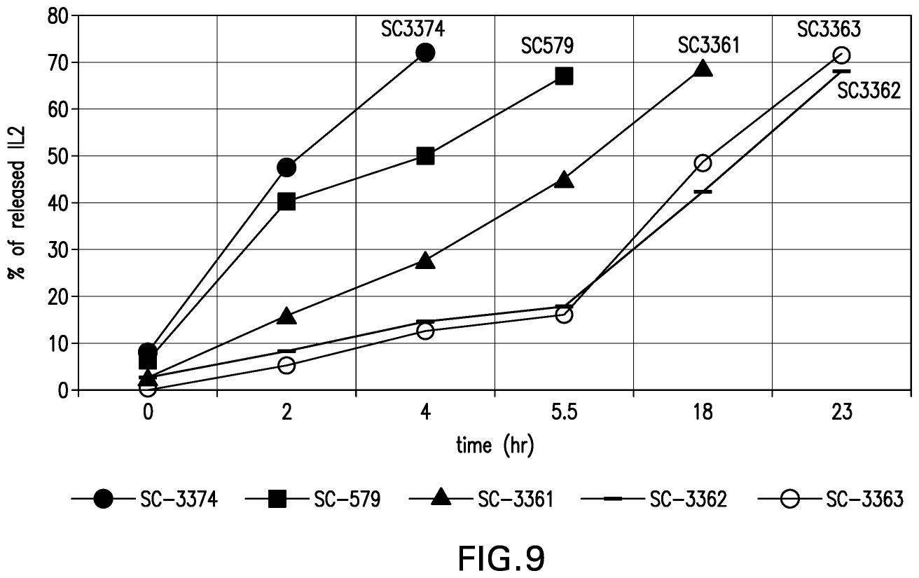

FIG. 8 shows the release-rate of PEG Fmoc linkers SC579, SC3361, SC3362, SC3363, and SC3374 conjugated to the S4-pAMF of aldesleukin variant SP9954. FIG. 9 shows the percentage of released aldesleukin variant SP9954 over time. The Release rate from fast to slowest was SC3374>SC579>SC3361>SC3362=SC3363. These results indicate that the medium release rate of the SC579 linker would provide a desirable balance between the prodrug state and the active state.

### Example 12

### Kit225STAT5-luc Assay

The human T lymphocyte Kit225 cell line (Hori et al., Blood 70:1069-1072 (1987)) was engineered with a STAT5 responsive luciferase reporter using the Promega pGL4.52 luc2P/STAT5 RE/Hygro vector (GenBank accession number JX206457). This vector contains five copies of a STAT5 response element (STAT5 RE) that drives transcription of the luciferase reporter gene luc2P. Kit225—STAT5Luc cells were maintained in RPMI-1640 with 10% heat-inactivated FBS, 0.6 mg/mL Hygromycin B, 1× GlutaMax, 1× Pen/Strep, and 10 ng/ml IL-2. To assess potency of IL-2 molecules, Kit225 STAT5luc cells were re-suspended at 0.5×10⁶ viable cells/mL in IL-2-free RPMI 1640 with 10% heat-inactivated FBS, 1× GlutaMax, and 1× Pen/Strep. Then, 25 μL cells were added per well into tissue culture-treated 384-well plates (Greiner) and incubated for five hours at 37° C. prior to addition of 25 μL of serial dilution of IL2 variants. Cells were then incubated overnight at 37° C., 5% CO₂. BrightGlo® from Promega were prepared according to manufacturer's instructions and 30 μL per well was added directly to treated cells. Relative luminescence was measured on an ENVISION® plate reader (Perkin Elmer; Waltham, MA). Relative luminescence readings were converted to % signal using untreated cells as controls. Data was fitted with non-linear regression analysis, using log (inhibitor) vs. response, variable slope, 4-parameter fit equation using GraphPad Prism. Data are shown in Examples 17 and 18.

### Example 13

Phosphorylated STAT5 (pSTAT5) Peripheral Blood Mononuclear Cell (PBMC) Assays

Cryopreserved human PBMCs were thawed in a 37° C. water bath and washed once with complete RPMI-1640 media (Corning) containing 10% heat-inactivated FBS (Sigma), 1× Pen/Strep (Corning), and 1× GlutaMax® (Gibco) to remove residual DMSO. Cells were then washed once with DPBS. A 1:1000 dilution of eFluor™ 780 fixable viability dye (Thermo-Fisher #65-0865-14) in DPBS was added and cells were incubated for 10 minutes in the dark at room temperature. Cells were then washed once with complete RPMI-1640, then washed again with DPBS. Cells were resuspended at 20×10⁶ cells/mL in DPBS and 50 μL cells per well were plated in a U-bottom 96-well plate (Greiner). Then, 50 μL of IL-2 sample was then added to cells and allowed to incubate for 15 minutes at 37° C. IL2 signalling was stopped by adding 100 μL of pre-warmed Cytofix® Fixation Buffer (BD) to cells and incubating for 10 minutes at 37° C. Cells were centrifuged, and the supernatant removed, followed by two washes in Fluorescence-activated cell sorting (FACS) buffer containing 1×PBS, 0.5% BSA, and 0.1% sodium azide. Fixed cells were then Fc-blocked (Biolegend #422302) for 30 minutes on ice. Surface stains (CD4, CD8, CD3, CD25 and CD127) were then applied and cells were incubated on ice for one hour. Cells were then washed once with FACS buffer and another time with DPBS. Ice-cold Perm Buffer III (BD #558050) was added dropwise to cells and then incubated on ice for 30 minutes. Perm Buffer III was then removed, and cells were washed twice with FACS buffer. Anti-pSTAT5 antibody (BD #562077) was applied to cells at a 1:50 dilution and incubated for one hour on ice. Cells were washed with FACS buffer, then lightly in 2% PFA for 10 minutes at room temperature. Cells were then washed and resuspended in FACS buffer, then and read on the AttuneNxT® flow cytometer and analysed by FlowJo® and GraphPad PRISM. Data are shown in Examples 17 and 18.

### Example 14

### In Vivo Activity of Aldesleukin Prodrug Variants in Syngeneic Mouse Model B16F10

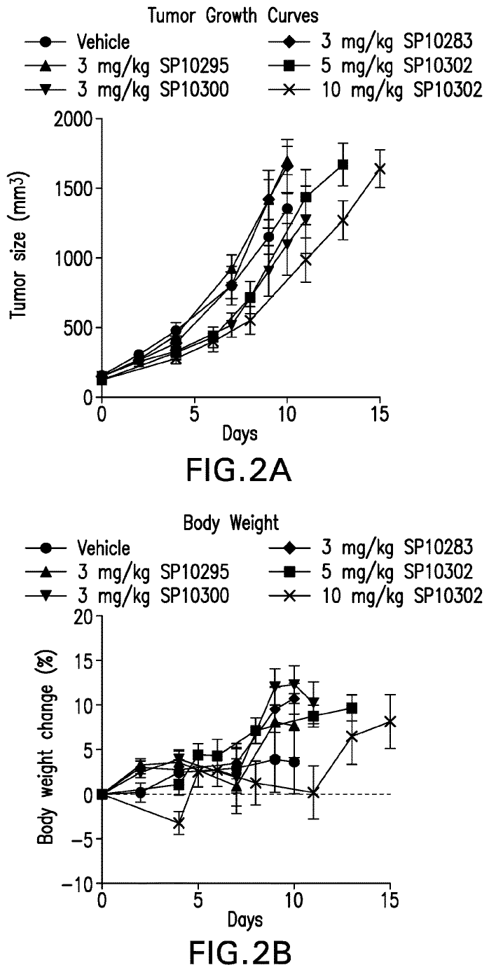

The anti-tumor activity of aldesleukin prodrug variants was evaluated in the syngeneic mouse melanoma tumor B16F10. In all studies, female C57BL/6 mice eight to ten weeks of age were anesthetized with isoflurane and implanted subcutaneously with 1×10⁶ B16F10 cells into the right hind flank. Randomization and start of treatment (n=9 per group) was initiated when tumors are established (average tumor size approximately 125 mm³). Body weight and tumor size were monitored 2×-3×/week until the mean of the vehicle control group was greater than 1,200 mm³. Animals with established B16F10 tumors were treated every seven days for 2 or 3 doses with the indicated doses of aldesleukin prodrug variants. The variants had increasing number of PEGs (SC578 or SC579) ranging from 20K-60K total PEG size, and all had the F41 conjugation site in common. FIGS. 2A and 2B show that all the variants tested did not elicit any meaningful efficacy or body weight loss in B16F10 tumors.

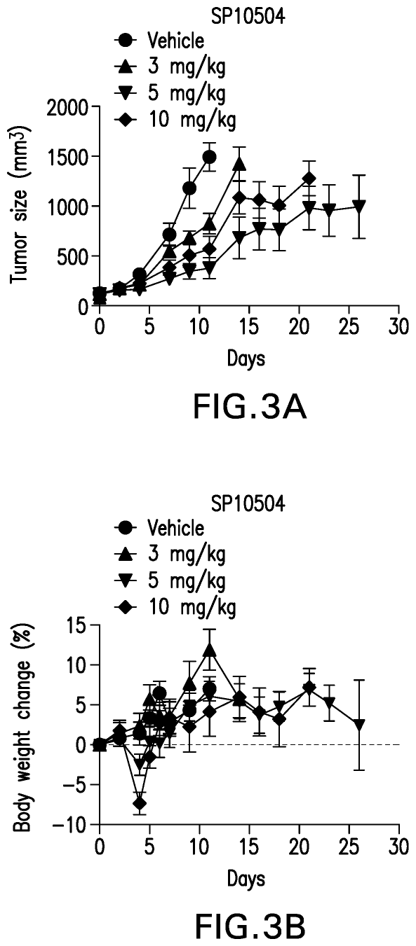

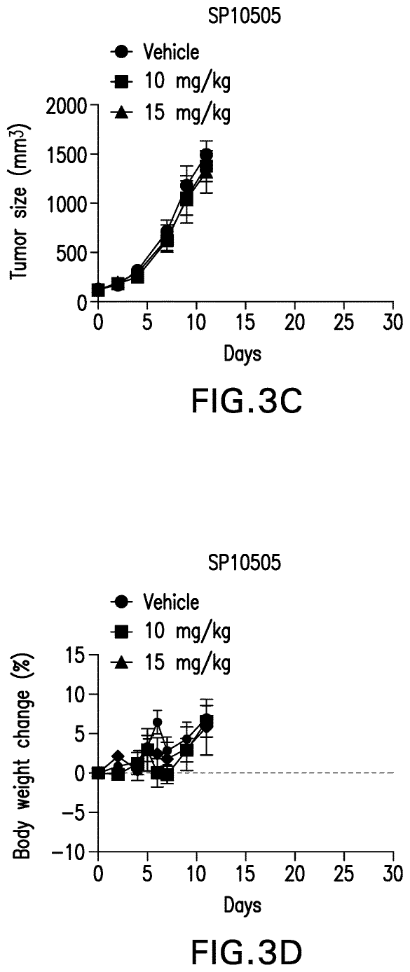

A subsequent study was performed to assess the activity of aldesleukin prodrug variants with and without the R37AF41K mutations that abrogate binding to IL2-Rα. SP10504 without the R37AF41K mutations showed minimal body weight loss and exhibited significant activity on Day 9 (81% TGI) and plateaued at 5 mg/kg (FIGS. 3A and 3B). No activity or body weight loss was observed for SP10505 with the R37AF41K mutations (FIGS. 3C and 3D), suggesting that IL2-Rα binding contributes to efficacy. These results are consistent with poor or lack of activity observed with prodrug variants with PEGs conjugated at the F41 site (FIG. 2A). Data are shown in Table 8.

TABLE 8
Efficacy

| | | | | | | Mean % |
|---|---|---|---|---|---|---|
| | | | | Dose | TGI | BWL @ |
| SP# | Site | Mutation^{†} | SC | (mg/kg) | (%) | nadir |
| 10295 | F41 | | 578 | 3 | 0% | None |
| 10300 | F41K34 | | 578 | 3 | 20% | None |
| 10283 | F41Y30K34 | | 578 | 3 | 0% | None |
| 10302 | F41Y30K34 | | 579 | 5 | 5% | None |
| 10302 | F41Y30K34 | | 579 | 10 | 37% | 3% |
| 10504 | S4Y30K34 | | 579 | 3 | 48% | None |
| 10504 | S4Y30K34 | | 579 | 5 | 81% | 3% |
| 10504 | S4Y30K34 | | 579 | 10 | 67% | 7% |
| 10505 | S4Y30K34 | R37A/ | 579 | 10 | 8% | 0.2% |
| | | F41K | | | | |
| 10505 | S4Y30K34 | R37A/ | 579 | 15 | 12% | None |
| | | F41K | | | | |
| 10784 | S4Y30K34Q73 | | 579 | 2 | 41% | None |
| 10784 | S4Y30K34Q73 | | 579 | 5 | 79% | 8% |
| 10784 | S4Y30K34Q73 | | 579 | 10 | 61% | >15% |
| 10784 | S4Y30K34Q73 | | 579 | 5 | 73% | 5% |
| 10922 | S4Y30K34Q73 | | 3361 | 4.22* | 39% | 3% |
| 10923 | S4Y30K34Q73 | | 3362 | 2.6* | 0% | None |

*exposure matched dose relative to 5 mg/kg SP10784
^{†}R37A/F41K double mutation abrogates affinity to IL2Rα and have been described in WO2021216478.

Comparison of SP10784 and Prodrug Variants with SC579 PEG Analogs

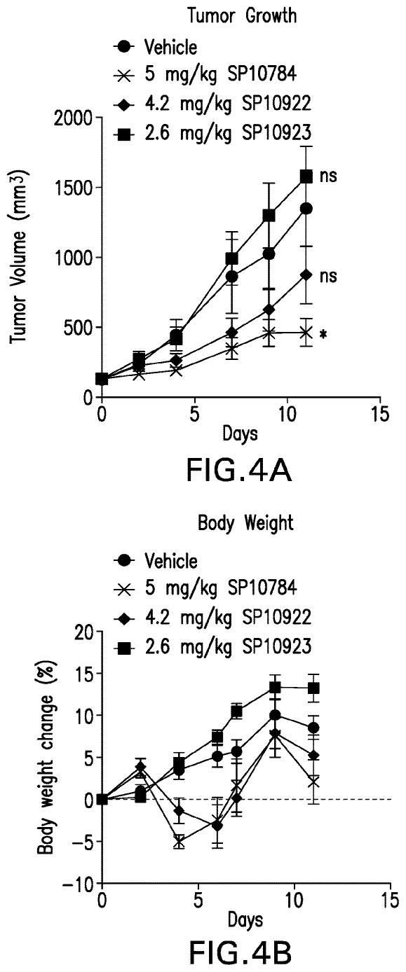

The efficacy of IL-2 variants with SC579 releasable PEG analogs was compared to SP10784 in animals bearing B16F10 tumors. Established B16F10 tumors (about 130 mm³) were treated every seven days for two total doses (q7dx2) with exposure matched doses of SP10784 (5 mg/kg), SP10922 (4.2 mg/kg) and SP10923 (2.6 mg/kg). SP10784 significantly inhibited B16F10 tumor growth (73% TGI) and was well tolerated (FIGS. 4A and 4B). Meanwhile, SP10922 and SP10923 showed poor activity, which were not statistically different compared to vehicle control (FIG. 4A). Therefore, SP10784 demonstrated superior activity compared to both PEG analog variants.

### Dose Response of SP10784

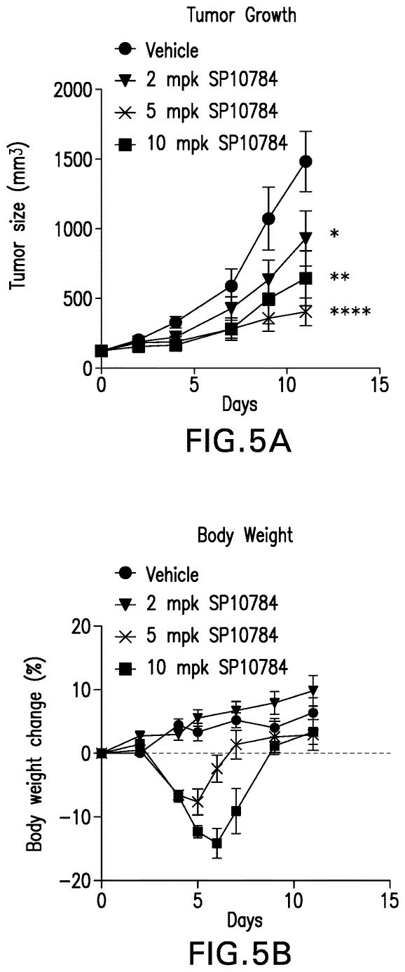

The dose-response relationship of SP10784 was assessed in B16F10 tumors. Female C57BL/6 mice bearing established B16F10 tumors (about 125 mm³) were treated intravenously (IV) with 2, 5, and 10 mg/kg SP10784 every seven days for two doses (q7dx2). Results showed that SP10784 exhibited dose-dependent anti-tumor activity and body weight loss (FIGS. 5A and 5B). Animals that received the lowest dose of 2 mg/kg SP10784 showed normal body weight gain and achieved moderate but significant activity (41% TGI). Treatment with 5 mg/kg SP10784 showed robust activity (79% TGI), but resulted in transient 10% body weight loss with recovery within two days. The highest dose (10 mg/kg SP10482) was only administered once due to severe body weight loss (>20%) resulting in 4/10 animal deaths. The remaining animals in this group achieved significant efficacy (61% TGI) with only a single dose of 10 mg/kg SP10784.

### Example 15

### Pharmacodynamic (PD) Effects of SP10784 in B16F10-Tumor-Bearing Mice

The PD effects of SP10784 on tumor-infiltrating immune cell subtypes was assessed using the B16F10 model. Briefly, C56BL/6 mice bearing established B16F10 tumors (around 140 mm³) were treated with a single intravenous dose of 5 mg/kg SP10784 or vehicle. Tumors were then collected on days 3, 7, and 10 post-treatment and processed for flow cytometry to establish a time course of the developing immune response.

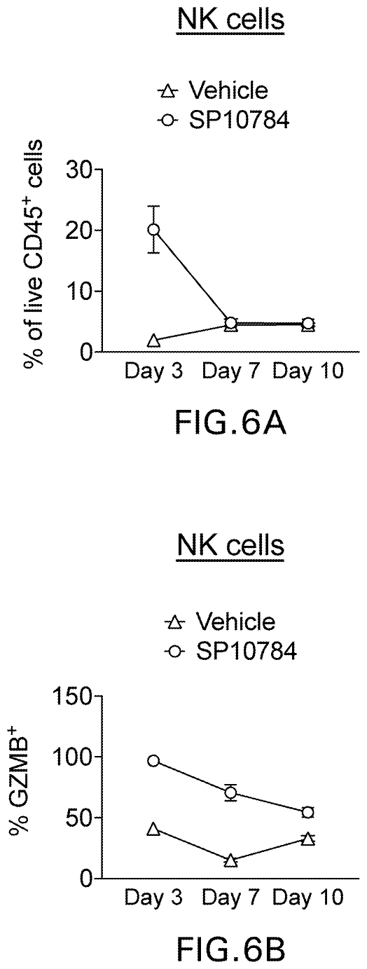

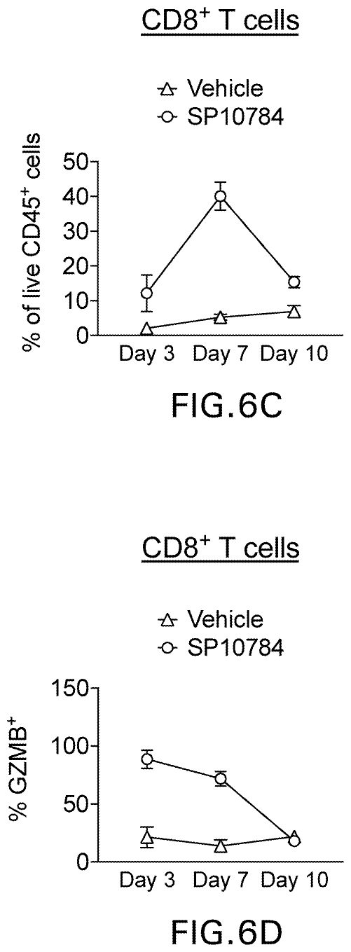

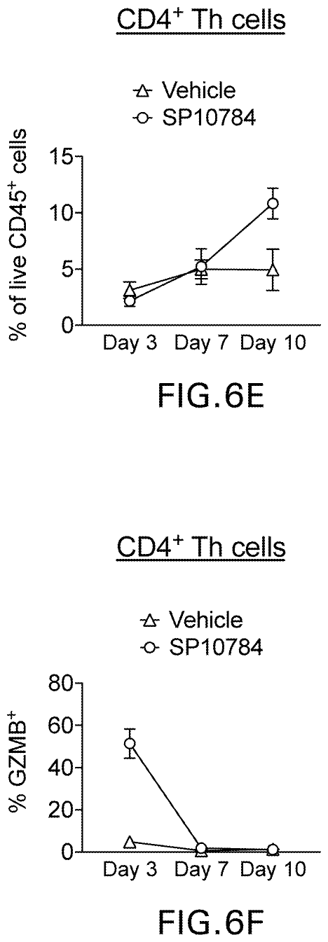

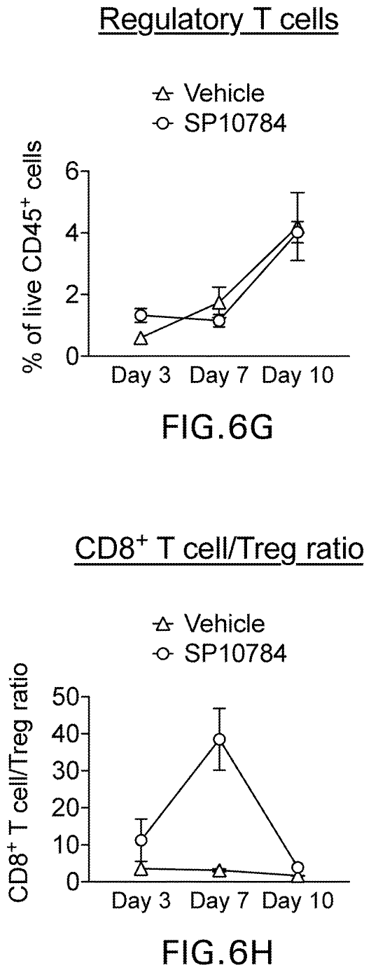

Examination of the tumor immune microenvironment revealed robust increases in the frequency of tumor-infiltrating natural killer (NK) cells and the proportion of granzyme-B-positive (GZMB⁺) NK cells following SP10784 treatment (FIG. 6A and FIG. 6B). For both these parameters, the maximal effect was observed at day three post treatment, with NK cell infiltration returning to baseline at day seven and the percent of GZMB⁺ NK cells returning near to baseline by day 10. CD8⁺ T cells similarly exhibited an increased frequency and an increased proportion of GZMB⁺ cells following SP17084 treatment (FIG. 6C and FIG. 6D). However, whereas GZMB⁺ CD8⁺ T cells showed a maximal increase at day three which returned to baseline by day 10, the increased frequency of CD8⁺ T cells exhibited a maximal effect at day seven and was present at all time points compared to vehicle control, indicating different kinetics for CD8⁺ T cell infiltration and activation. Frequencies of CD4⁺ T-helper (Th) cells—defined as CD4⁺ FOXP3⁻ T cells—in the tumor showed no difference between SP10784-treated and vehicle-treated mice at day three and day seven but show an increased frequency with SP10784 treatment at day 10 (FIG. 6E). A robust but transient increase in GZMB⁺ Th cells was observed at day 3 in SP10784-treated mice but returned to baseline by day seven (FIG. 6F). Regulatory T cells (T_{regs})—defined as CD4⁺ FOXP3⁺ T cells—showed a slight difference in frequency at day 3 post treatment but was similar to vehicle control at day seven and ten (FIG. 6G). Given these changes in the frequencies of tumor-infiltrating CD8⁺ T cells and T_{regs}, the CD8⁺ T cell/T_{reg} ratio in the tumor was increased at all time points following SP10784 treatment, with a maximal ratio observed at day seven (FIG. 6H).

These PD effects in the tumor immune microenvironment are consistent with the potent anti-tumor efficacy observed following SP10784 treatment in the B16F10 model. The increased infiltration and activation of effector cells, such as NK cells and CD8⁺ T cells, indicate potentiation of the anti-tumor immune response with SP10784 treatment, and the increased CD8⁺ T cell/Treg ratio further supports this. Overall, FIGS. 6A-6H show that SP10784 increases CD8⁺ T cell and NK cell frequency and cytotoxic potential in the B16F10 tumor microenvironment.

### Example 16

### In Vivo Pharmacokinetic (PK) Assessment of Prodrug Aldesleukin Variants

The pharmacokinetic (PK) profile of IL-2 prodrug variants was evaluated by total antibody levels following a single 0.8 mg/kg IV bolus in non-tumor bearing C57BL/6 mice. Plasma samples were collected at several time points up to seven days for composite PK analysis (n=3 per time point). Samples were processed to allow PEG release followed by ELISA analysis to determine plasma concentrations of variant IL-2 species. Aldesleukin (wild-type IL-2) was included in all data sets for comparison to the prodrug variants.

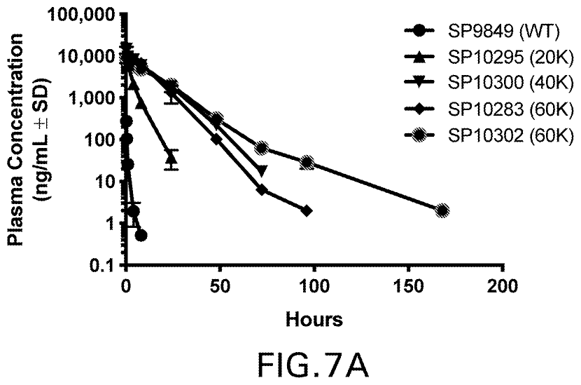

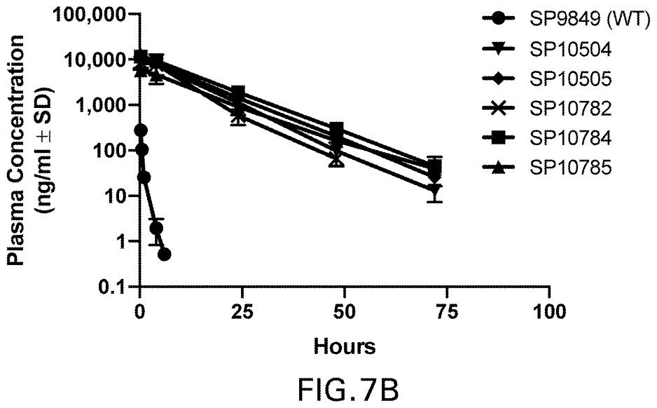

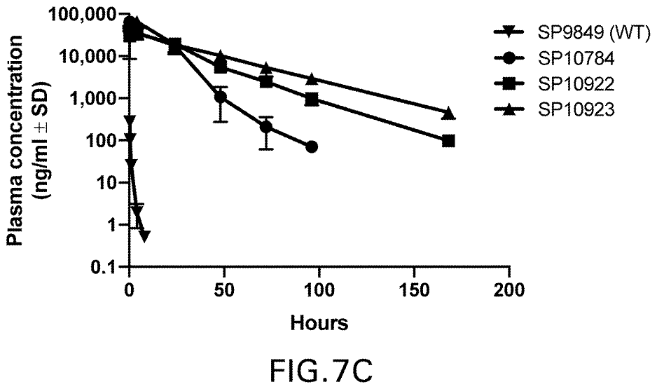

PK analysis demonstrated that all prodrug aldesleukin variants have prolonged half-lives (T_{1/2}) and exposure (increase area under the curve, AUC) compared to wild-type IL-2. FIG. 7A shows IL-2 variants with increasing number of releasable PEGs with 20 kDa to 60 kDa total PEG. Of all the compounds, SP10295 conjugated to the least amount of PEG (20 kDa) showed the shortest half-life, lowest exposure, and highest clearance (CL) compared to the other variants (Table 9).

TABLE 9
Summary of PK parameters from C57BL/6 mice treated with aldesleukin prodrug variants
with increasing number of PEGs

| | T_{1/2} | C₀ | Cₘₐₓ | Clearance | AUC 0-last | AUC 0-inf | Vss |
|---|---|---|---|---|---|---|---|
| SP# | (hrs) | (ng/mL) | (ng/mL) | (mL/hr/kg) | (hr*ng/mL) | (hr*ng/mL) | (mL/kg) |
| 9849 | 2.1* | 734 | 276 | 3,150 | 253 | 254 | 1,660 |
| 10295 | 3.5 | 14,200 | 11,600 | 22.8 | 34,800 | 35,000 | 76.3 |
| 10300 | 7.7 | 16,900 | 14,700 | 5.0 | 158,000 | 159,000 | 57.0 |
| 10283 | 7.2 | 19,600 | 16,500 | 5.2 | 155,000 | 155,000 | 48.3 |
| 10302 | 17.2 | 9,680 | 9,250 | 5.7 | 141,000 | 141,000 | 84.0 |

*Estimate only based on the last two timepoints.

The 60 kDa variant using the linear PEG SC579 (SP10302) appeared to have the best PK profile and longest half-life, but the other PK parameters were not significantly different from SP10283 or SP10300 (Table 9). Additional IL-2 prodrug variants with 60 kDa or 80 kDa total PEG (utilizing SC579), including SP10784, had similar terminal half-lives and CL ranging from 7.4-9.6 hours and 4.9-8.6 mL/hr/kg, respectively (FIG. 7B and Table 10).

TABLE 10
Summary of PK parameters from C57BL/6 mice treated with aldesleukin prodrug variants
at different conjugation sites

| | T_{1/2} | C₀ | Cₘₐₓ | Clearance | AUC 0-last | AUC 0-inf | Vss |
|---|---|---|---|---|---|---|---|
| SP# | (hrs) | (ng/mL) | (ng/mL) | (mL/hr/kg) | (hr*ng/mL) | (hr*ng/mL) | (mL/kg) |
| 9849 | 2.1* | 734 | 276 | 3,150 | 253 | 254 | 1,660 |
| 10504 | 7.4 | 10,900 | 10,600 | 5.8 | 138,000 | 138,000 | 45.3 |
| 10505 | 8.2 | 13,100 | 12,700 | 4.9 | 161,000 | 161,000 | 44.0 |
| 10782 | 6.4 | 8,630 | 8,580 | 6.4 | 125,000 | 125,000 | 38.6 |
| 10784 | 8.9 | 11,800 | 11,700 | 4.3 | 185,000 | 186,000 | 43.6 |
| 10785 | 9.9 | 5,850 | 5,770 | 8.6 | 92,000 | 92,500 | 91.1 |

*Estimate only based on the last two timepoints.

A comparison of IL-2 variants with different releasable linkers showed that SP10923 SP10922 and showed improved PK properties, specifically longer half-life, compared to SP10784 (FIG. 7C and Table 11). However, anti-tumor activity of SP10922 and SP10923 were inferior to exposure matched doses of SP10784 (FIG. 4A).

TABLE 11
Summary of PK parameters from C57BL/6 mice treated with aldesleukin prodrug variants
with different PEG analogs

| | T_{1/2} | C₀ | Cₘₐₓ | Clearance | AUC 0-last | AUC 0-inf | Vss |
|---|---|---|---|---|---|---|---|
| SP# | (hrs) | (ng/mL) | (ng/mL) | (mL/hr/kg) | (hr*ng/mL) | (hr*ng/mL) | (mL/kg) |
| 9849 | 2.1* | 734 | 276 | 3,150 | 253 | 254 | 1,660 |
| 10784 | 8.6 | 67,000 | 64,500 | 0.810 | 985,000 | 987,000 | 10.4 |
| 10922 | 20.7 | 30,100 | 33,900 | 0.703 | 1,140,000 | 1,140,000 | 17.4 |
| 10923 | 27.0 | 64,200 | 65,400 | 0.427 | 1,850,000 | 1,870,000 | 12.4 |

*Estimate only based on the last two timepoints

In two independent studies, SP10784 demonstrated an extended and favorable PK profile compared to aldesleukin (FIGS. 7B and 7C), while maintaining robust anti-tumor activity in the B16F10 model (FIG. 5A and FIG. 4A). The PK data for all IL-2 variants are also summarized in Table 12.

TABLE 12
Pharmacokinetics

| | PEGylation | | | Dose | T1/2 | Clearance | AUC 0-inf |
|---|---|---|---|---|---|---|---|
| SP# | Site(s) | Mutation^{†} | SC# | (mg/kg) | (hrs) | (mL/hr/kg) | (hr*ng/mL) |
| 10295 | F41 | | 578 | 0.8 | 3.50 | 22.8 | 35,000 |
| 10300 | F41K34 | | 578 | 0.8 | 7.73 | 5.04 | 159,000 |
| 10283 | F41Y30K34 | | 578 | 0.8 | 7.21 | 5.18 | 155,000 |
| 10302 | F41Y30K34 | | 579 | 0.8 | 17.2 | 5.69 | 141,000 |
| 10504 | S4Y30K34 | | 579 | 0.8 | 7.36 | 5.79 | 138,000 |
| 10505 | S4Y30K34 | R37A/F41K | 579 | 0.8 | 8.22 | 4.96 | 161,000 |
| 10782 | S4K34V114 | | 579 | 0.8 | 6.4 | 6.4 | 125,000 |
| 10784 | S4Y30K34Q73 | | 579 | 0.8 | 8.9 | 4.3 | 186,000 |
| 10785 | S4Y30K34V114 | | 579 | 0.8 | 9.9 | 8.6 | 92,500 |
| 10784 | S4Y30K34Q73 | | 579 | 0.8 | 8.6 | 0.810 | 987,000 |
| 10922 | S4Y30K34Q73 | | 3361 | 0.8 | 20.7 | 0.703 | 1,140,000 |
| 10923 | S4Y30K34Q73 | | 3362 | 0.8 | 27.0 | 0.427 | 1,850,000 |

^{†}R37A/F41K double mutation abrogates affinity to IL2Rα and have been described in WO2021216478.

### Example 17

### In Vitro Pharmacology

Label-Free Kinetic Analysis with Surface Plasmon Resonance (SPR)

To understand the binding properties of the IL-2 prodrug before and after PEG release, kinetic binding to IL-2 receptors was evaluated with PEG-conjugated and “stump”—conjugated IL-2 variants. Released PEG from SC578 and SC579 leave a “stump” at the site of conjugation, consisting of the DBCO-triazole moiety and some remaining linker sequence. A fully revealed IL-2 variant where all PEG molecules have been released can be represented by the conjugation of SC547 which constitutes the conjugation stump.

Multi-pAMF incorporated IL-2 variants were conjugated to a 20 kDa releasable PEG (SC578 or SC579) or to a “stump” (SC547), and their kinetic binding was assessed to the IL-2 receptors IL-2Rα and IL-2Rβ. As expected, IL-2 variants masked by the conjugation of multiple PEGs had undetectable or significantly attenuated binding to both IL-2 receptors (Table 13, not detected or not calculable). However, IL-2 variants conjugated to a stump molecule had varying levels of activity. Some variants had attenuated binding to IL-2Rβ (e.g. SP10721-SP10725, SP10727-10728), and others maintained binding similar to Ald-6HIS (e.g. SP10504, SP10726, SP10782, SP10784, SP10785).

TABLE 13

| | | | | Stump conjugate | | | PEG conjugate | | |
|---|---|---|---|---|---|---|---|---|---|
| | PEGylation | | His | SC | IL- | IL- | SC | IL- | IL- |
| SP# | Site(s) | Mutation^{†} | Tag | # | 2Rα | 2Rβ | # | 2Rα | 2Rβ |
| 10295 | F41 | | | 547 | NT | NT | 578 | NT | NT |
| 10300 | F41K34 | | | 547 | NT | NT | 578 | NT | NT |
| 10283 | F41Y30K34 | | | 547 | NT | NT | 578 | NT | NT |
| 10302 | F41Y30K34 | | | 547 | NT | NT | 579 | NT | NT |
| 10504 | S4Y30K34 | | | 547 | NC | 7E−06 | 579 | NC | NC |
| 10505 | S4Y30K34 | R37A/F41K | | 547 | NT | NT | 579 | NT | NT |
| 10721 | S4K34Q73 | | Y | 547 | 2E−08 | 1E−06 | 579 | ND | ND |
| 10722 | S4K34F77 | | Y | 547 | 4E−08 | 3E−06 | 579 | NC | ND |
| 10723 | S4K34V114 | | Y | 547 | 3E−07 | 2E−06 | 579 | NT | NT |
| 10724 | S4Y30K34 | F41K | Y | 547 | NC | 3E−06 | 579 | ND | ND |
| 10725 | S4Y30K34R80 | | Y | 547 | NC | NC | 579 | ND | ND |
| 10726 | S4Y30K34Q73 | | Y | 547 | 3E−08 | 6E−07 | 579 | ND | ND |
| 10727 | S4Y30K34V114 | | Y | 547 | 1E−07 | 1E−06 | 579 | ND | ND |
| 10728 | S4Y30K34R80 | F41K | Y | 547 | NC | 2E−06 | 579 | ND | ND |
| 10782 | S4K34V114 | | | 547 | 1E−07 | 5E−07 | 579 | NC | ND |
| 10784 | S4Y30K34Q73 | | | 547 | 9E−09 | 3E−07 | 579 | 2E−06 | NC |
| 10785 | S4Y30K34V114 | | | 547 | NC | 2E−07 | 579 | NC | ND |

NC = not calculable;
ND = not detected;
NT = not tested
^{†}R37A/F41K and F41K mutations abrogate or reduce, respectively, affinity to IL2Rα and have been described in WO2021216478.

Binding affinities to IL-2Rα were more variable, consistent with previous data that some of these sites for pAMF incorporation lie in the IL-2Rα binding interface, suggesting that either the pAMF alone or a stump-conjugated to pAMF may interfere with IL-2Rα engagement. This analysis suggests that a number of IL-2 prodrugged variants-once revealed-would maintain activity and signaling via the IL-2Rβ and IL-2Rγ complex to a similar level as Ald-6HIS. In the presence of PEG, these IL-2 variants were significantly prodrugged as they maintained limited or no binding to IL-2 receptor complexes. As such, the pegylated variants would be predicted to have limited activity when first dosed, reducing the likelihood of systemic toxicities. Depending on the desired product profile, some IL-2 conjugates maintained IL-2Rα binding while others did not.

### Example 18

This example shows the EC₅₀ for various aldesleukin variants conjugated to a releasable Fmoc PEG as determined using the previously described pSTAT5, STAT5, Kit225, NK-92, Derl-7 Assays. Table 14 shows the results.

TABLE 14
EC₅₀ (nM) for pSTAT5, STAT5, Kit225, NK-92, Derl-7 Assays

| | PEGylation | | | Kit225- | NK- | | pSTAT5 | pSTAT5 |
|---|---|---|---|---|---|---|---|---|
| SP# | Site(s) | SC# | Mutation^{†} | STAT5 | 92 | DERL7 | Treg | CTL |
| SP10295 | F41 | SC578 | — | NT | 0.09 | 0.10 | NT | NT |
| SP10300 | F41K34 | SC578 | — | NT | 0.41 | 0.40 | NT | NT |
| SP10283 | F41Y30K34 | SC578 | — | 15.87 | 1.38 | 1.53 | >1000 | >1000 |
| SP10302 | F41Y30K34 | SC579 | — | 1.19 | 2.38 | 4.47 | NT | NT |
| SP10504 | S4Y30K34 | SC579 | — | 0.04 | 0.17 | 16.43 | 15.60 | >1000 |
| SP10505 | S4Y30K34 | SC579 | R37A/F41K | 4.13 | 5.25 | 9.96 | >1000 | >1000 |
| SP10721 | S4/K34/Q73 | SC579 | — | 0.06 | 0.21 | 17.40 | 16.8 | >1000 |
| SP10722 | S4/K34/F77 | SC579 | — | 0.11 | 0.30 | 20.90 | 20.9 | >1000 |
| SP10723 | S4/K34/V114 | SC579 | — | 2.71 | 4.69 | 48.50 | >200 | >1000 |
| SP10724 | S4/Y30/K34 | SC579 | F41K | 9.49 | 13.20 | 25.70 | >1000 | >1000 |
| SP10725 | S4/Y30/K34/ | SC579 | — | 0.33 | 0.99 | 24.80 | ~255 | >1000 |
| | R80 | | | | | | | |
| SP10726 | S4/Y30/K34/ | SC579 | — | 0.39 | 1.14 | 65.10 | >200 | >1000 |
| | Q73 | | | | | | | |
| SP10727 | S4/Y30/K34/ | SC579 | — | 4.78 | 11.50 | 74.30 | >1000 | >1000 |
| | V114 | | | | | | | |
| SP10728 | S4/Y30/K34/ | SC579 | F41K | 28.40 | 25.80 | 45.80 | >1000 | >1000 |
| | R80 | | | | | | | |
| SP10782 | S4/K34/V114 | SC579 | — | 0.13 | 0.54 | 7.84 | >1000 | >1000 |
| SP10784 | S4/Y30/K34/ | SC579 | — | 0.05 | 0.18 | 7.01 | >1000 | >1000 |
| | Q73 | | | | | | | |
| SP10785 | S4/Y30/K34/ | SC579 | — | 0.25 | 1.18 | 11.80 | >1000 | >1000 |
| | V114 | | | | | | | |

NT = not tested
^{†}R37A/F41K and F41K mutations abrogate or reduce, respectively, affinity to IL2Rα and have been described in WO2021216478.

### Example 19

### Single Dose Pharmacokinetics in Mice

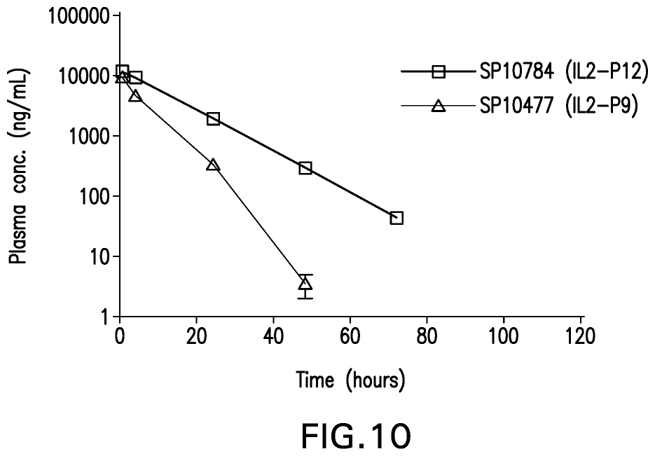

The pharmacokinetic (PK) profiles of SP10784 were evaluated by total IL-2 levels following a single IV dose of 0.8 mpk in non-tumor bearing C57BL/6 mice. Plasma samples were collected at several time points up to 5 days for PK analysis. The mean plasma concentration profiles of IL-2 variants SP10784 and SP10477 (IL-2 conjugated the non-releasable 30K PEG) were obtained from different studies and are included here for comparison purposes (FIG. 10). PK analysis showed that releasable PEG variants have a higher exposure, longer MRT, lower CL compared to the releasable PEG variant (Table 15).

TABLE 15
Summary of PEG IL-2 PK parameters in C57BL/6 mice

| Treat- | Descrip- | Cₘₐₓ | CL | AUC-INF | MRT | Vss |
|---|---|---|---|---|---|---|
| ment | tion | (ng/mL) | (mL/kg/h) | (h*ng/mL) | (h) | (mL/kg) |
| SP10784 | IL-2 | 11,667 | 4.3 | 185,801 | 9.9 | 43.7 |
| | Releasable | | | | | |
| | PEG | | | | | |
| SP10477 | IL-2 Non- | 9,523 | 9.6 | 83,640 | 4.9 | 47.1 |
| | releasable | | | | | |
| | PEG | | | | | |

Mean Residency Time (MRT), instead of half-life, is reported here to reflect the drug residency in the body more accurately.

### Example 20

### Repeat-Dose Pharmacokinetic (PK) and Pharmacodynamic (PD) Study of SP10784 in Non-Human Primates (NHP)

Pharmacokinetics (PK) studies, including pharmacodynamic (PD) endpoints, were conducted to characterize SP10784 PK/PD relationship in non-human primates (cynomolgus monkey and rhesus monkeys). The PD readouts were collected from blood at several timepoints and consisted of soluble CD25 (sCD25), immunophenotyping of peripheral lymphocyte populations and intracellular proliferation markers, in addition to inflammation and/or vascular leakage markers.

A repeat-dose cynomolgus monkey study (study #20295005) was pursued with the objective of characterizing SP10784 PKPD and early tolerability in comparison to SP10477 (PEG-IL-2 non-releasable variant). The study in rhesus monkeys (study #21-M100-18597) was performed to evaluate SP10784 PKPD after subcutaneous (SC) administration in support of toxicology studies.

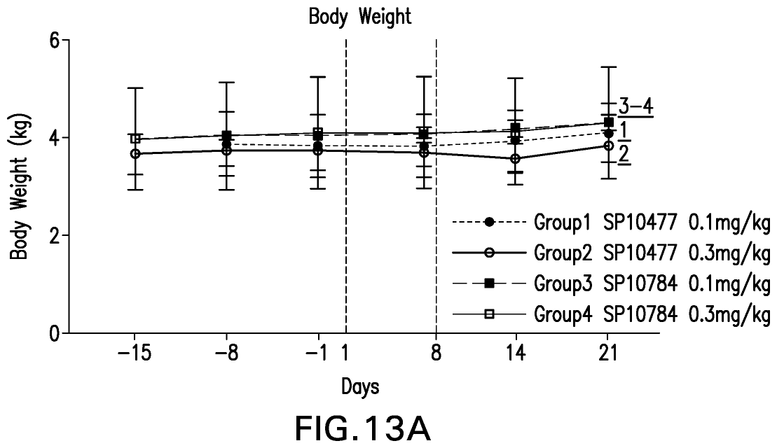

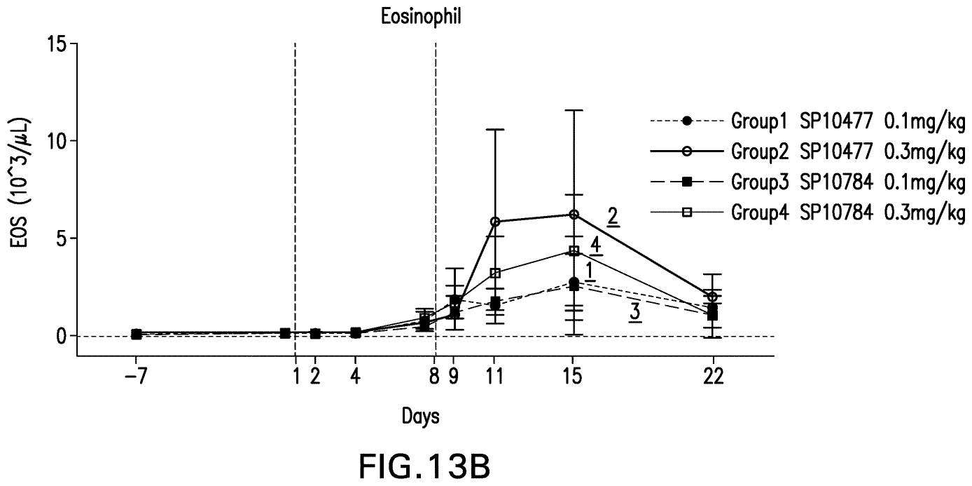

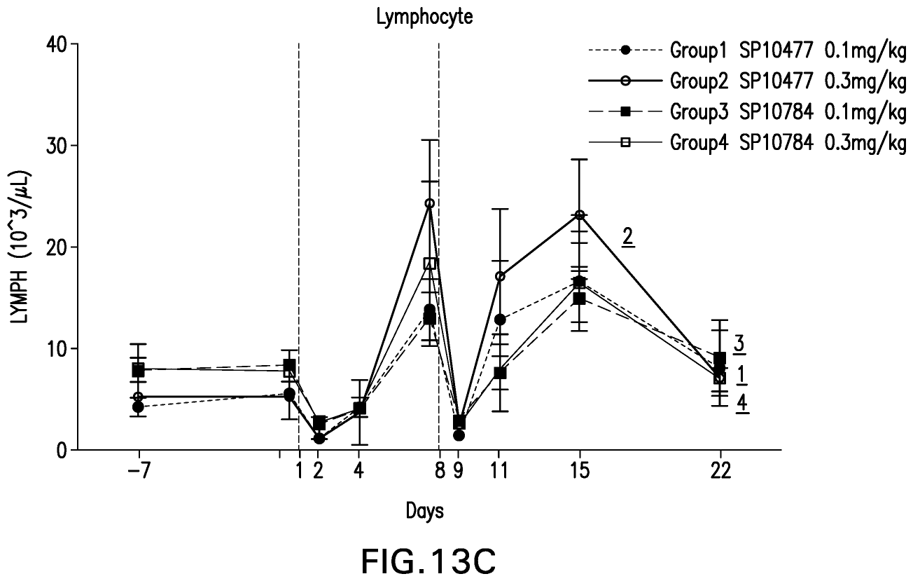

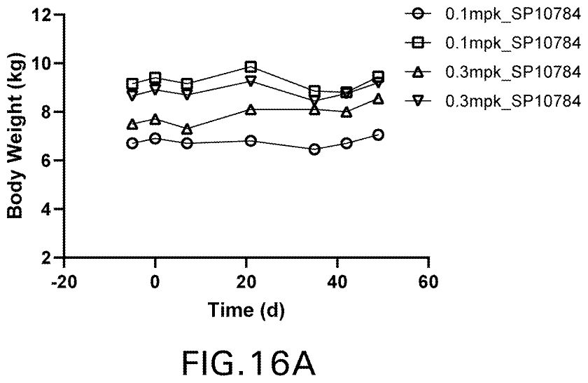

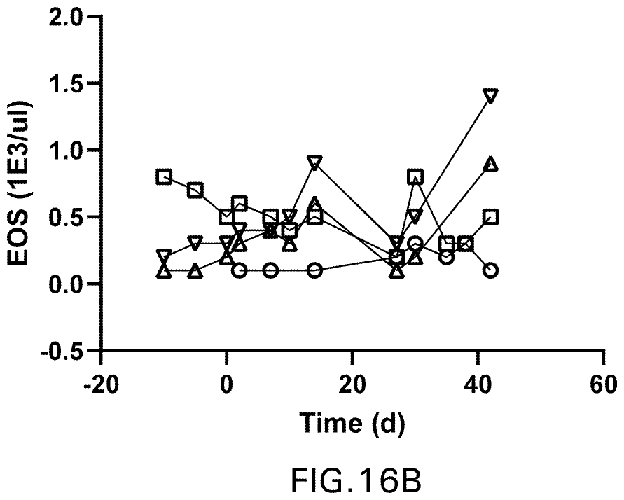

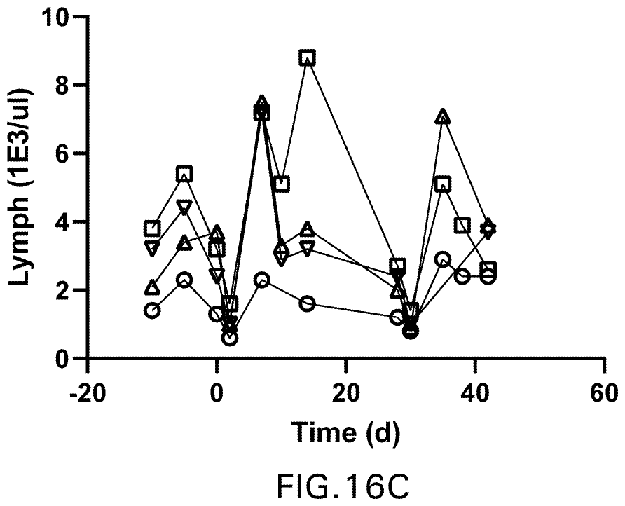

In summary, SP10784 was well tolerated at the doses tested (0.1 and 0.3 mpk) in cynomolgus money as repeat doses and in rhesus after single dose SC or IV. No changes in body weight were evidenced (FIG. 13A and FIG. 16A) and eosinophil counts increase following treatment returned to basal levels over time (FIG. 13B and FIG. 16B, cynomolgus monkey and rhesus monkey, respectively).

SP10784 resulted in pharmacodynamic effects that included rapid decreases (margination) in lymphocytes within 2 days post-dose, followed by concomitant lymphocyte increases (Days 4-8). These cyclical trends were observed throughout doses, molecules (FIG. 13C), and were comparable across route of administration (FIG. 16C) with evidence of reversibility to baseline levels by end of the study.

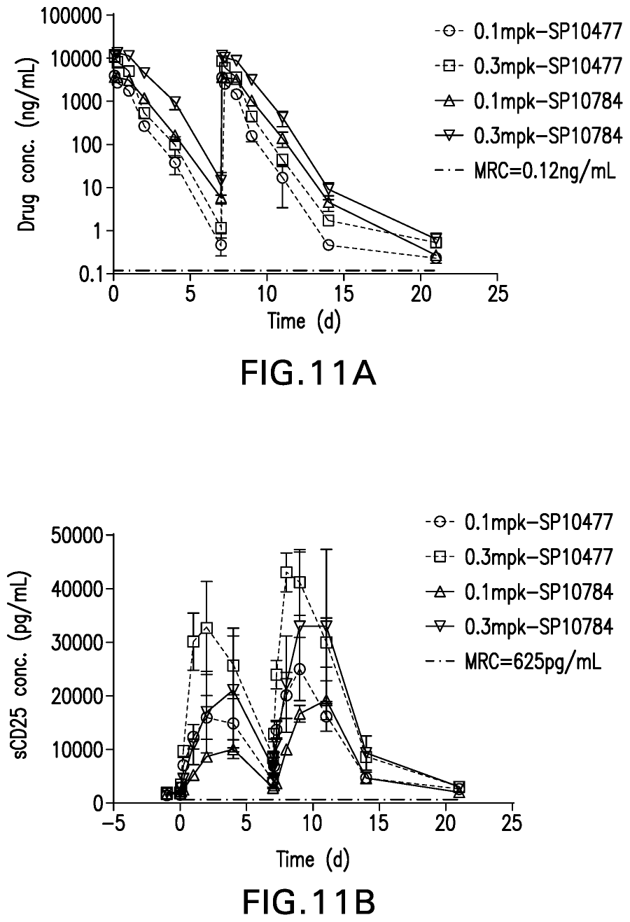

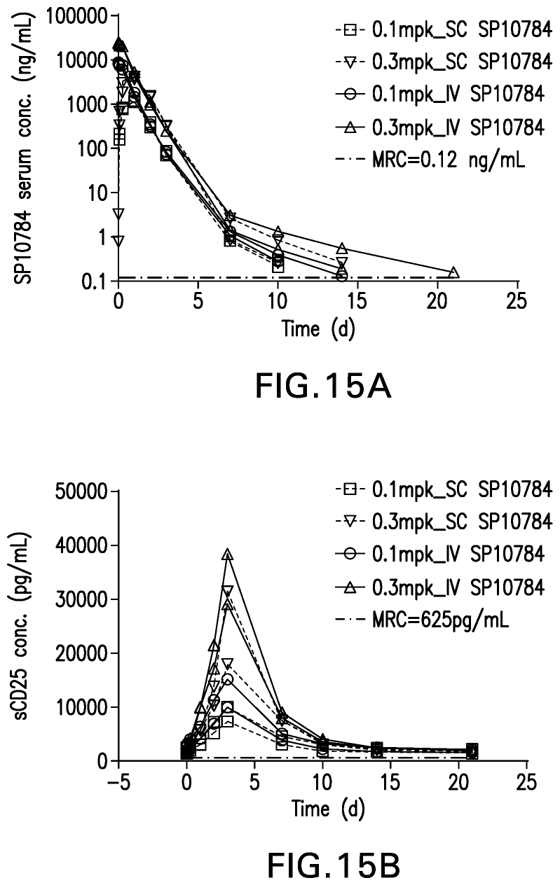

A protracted SP10784 systemic exposure (~2-fold MRT) and delayed sCD25 peak response relative to SP10477 was observed in the cynomolgus monkey study with a Tₘₐₓ of four days for SP10784 versus two days for the non-releasable PEG-IL2 variant (SP10477) (FIG. 11A). A dose dependent sC25 profile was observed in both NHP studies with a comparable response across routes of administration. A bioavailability of about 30% resulted from the SP10784 SC administration in rhesus monkey (FIG. 15A); however, the PD response was consistent across both, SC and IV, routes. Soluble sCD25 data is depicted in FIG. 11B and FIG. 15B, cynomolgus monkey and rhesus respectively.

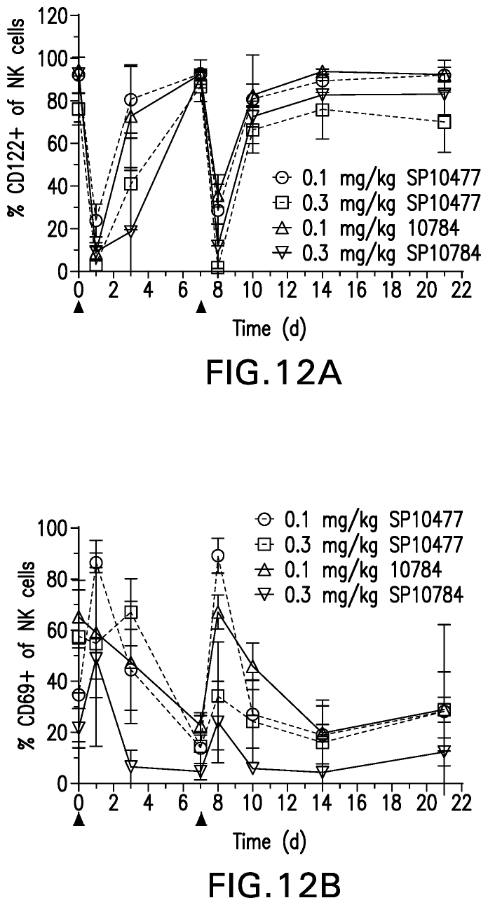

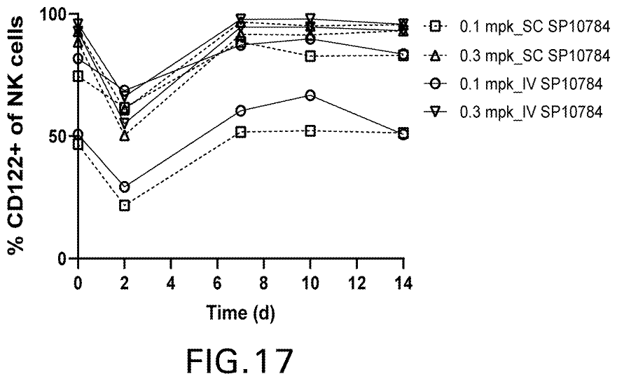

Loss of percent CD122 in NK cells in response to treatment at day 1 in cynomolgus monkeys and day 2 in rhesus monkeys was indicative of target engagement and the effect was comparable among SP10784 and the non-releasable PEG counterpart, SP10477 (FIG. 12A). In rhesus monkey, a dose dependent effect was observed, and the profile and magnitude of response was similar across ROA. The loss of percent CD122 response to SP10484 administered SC or IV at 0.1 or 0.3 mpk is shown in FIG. 17.

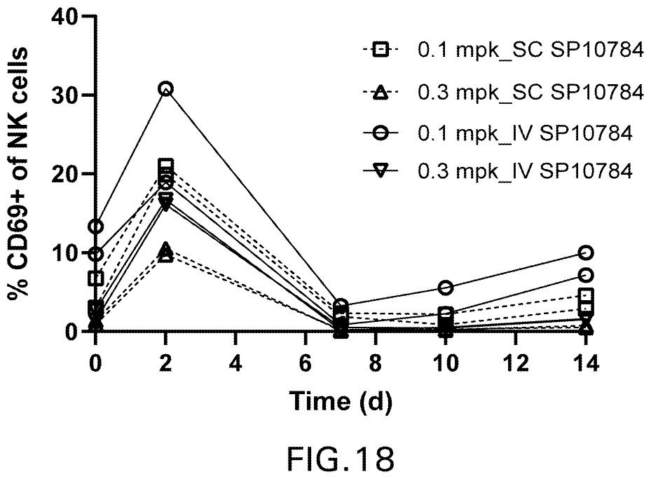

Percent of CD69 in NK cells, considered a surrogate for cell activation, increased in response to treatment, independent of dose level or ROA, and despite the IL-2PEG variants. However, SP10477 showed a higher magnitude of response (~1.5-fold) compared to that of SP10784 (FIG. 12B). The percent CD69 response to SP10484 administered SC or IV at 0.1 or 0.3 mpk is shown in FIG. 18.

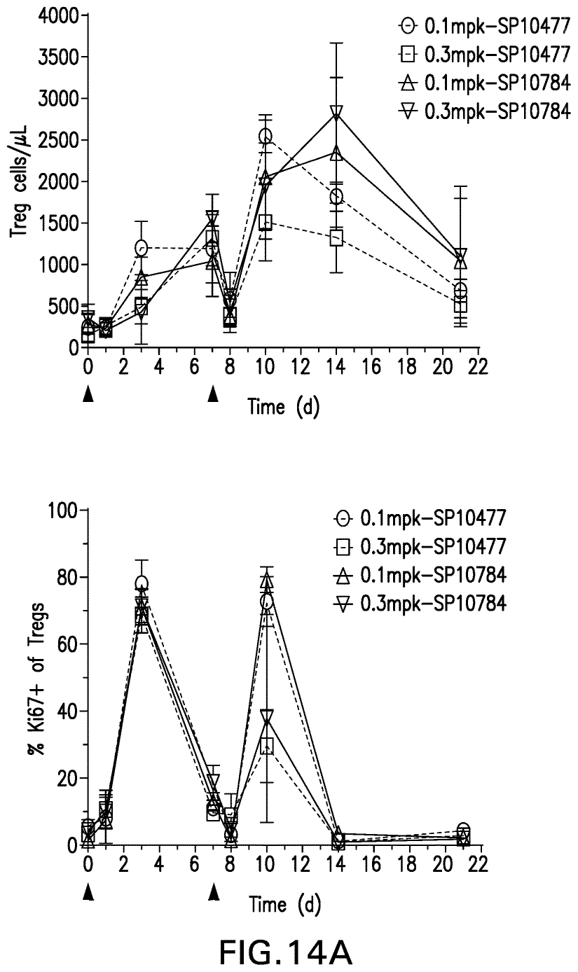

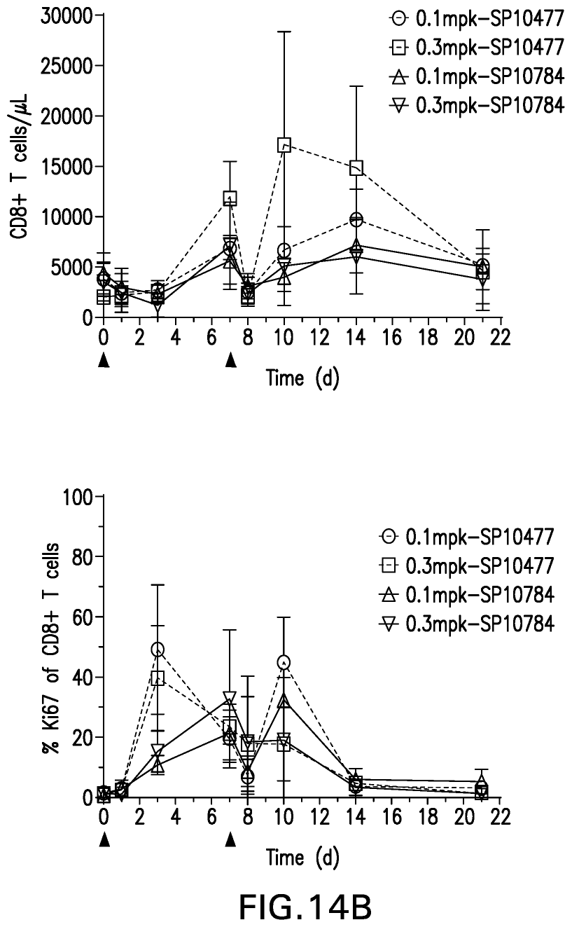

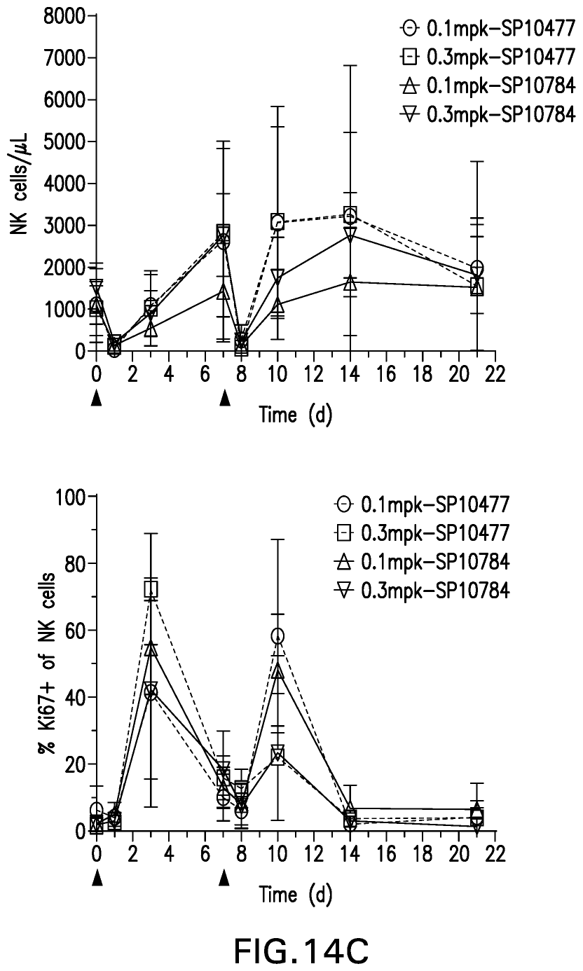

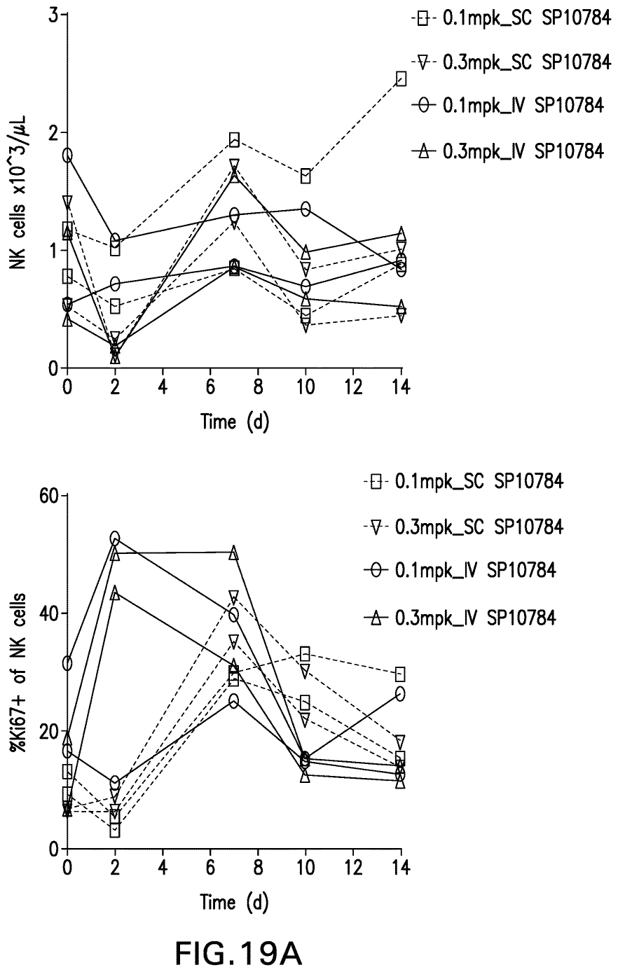

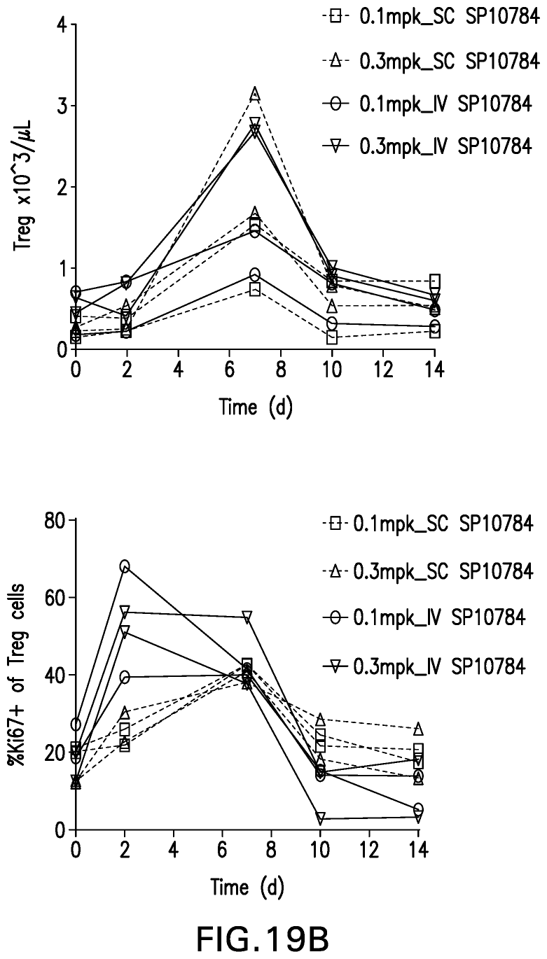

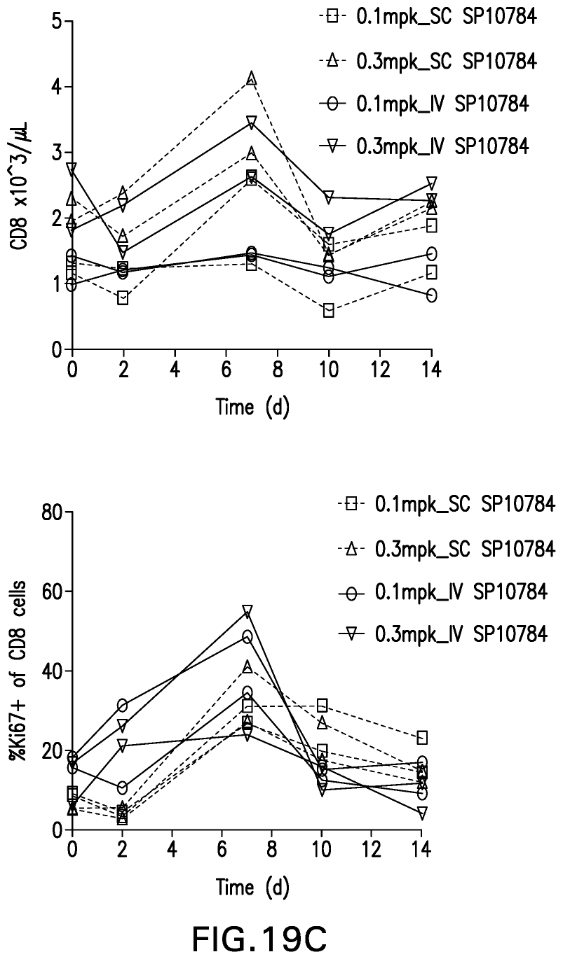

Immunophenotyping in the relevant lymphocyte population (NK, CD8⁺, and Tregs) revealed activation in response to SP10784 and this was comparable across the other IL-2PEG variants. Peak response was observed at 7 days post-dose and no clear dose level dependency or ROA impact was detected (FIG. 14A-14C, left panel, and FIG. 19A-19C, left panel).

No increase of CD8-to-T_{reg} ratios, relative to baseline, resulted from treatment with SP10784 or SP10477.

Results from the intracellular proliferation marker (Ki67) assessment showed a trend to dose dependency increase (FIG. 14A-14C, right panel, and FIG. 19A-19C, right panel). In the CD8 population a protracted max response was observed for SP10784 relative to SP10477 (7 days vs 3 days post-dose). Independent of treatment, an abrogation of the response was observed after the second 0.3 mpk dose (FIG. 13A-13C). A faster and higher peak response in NK and T_{reg} was observed after IV versus SC SP10784 administration (2 days vs. 7 days, respectively).

Overall, SP10784 PK in cynomolgus monkey and rhesus monkey were comparable, with an about 2-fold lower clearance observed in rhesus and a consistent PD response across both strains. A summary of SP10784 PK parameters is depicted in Table 16.

TABLE 16

| Study No. | | Dose | | | | | | |
|---|---|---|---|---|---|---|---|---|
| (species/ | | mpk | Cmax | CL | AUC-INF | F | MRT^{b} | Vss |
| strain) | Treatment | [ROA] | (ng/mL) | (mL/h/kg) | (h*ng/mL) | (%) | (h) | (mL/kg) |
| 20205005 | SP10784 | 0.1 | 3,706 ± | 1.7 ± | 79,625 ± | NA | 14.2 ± | 24.8 ± |
| (naïve | | [IV], | 442 | 0.2 | 10,244 | | 0.6 | 2.1 |
| cynomolgus) | | 1^{st} | | | | | | |
| monkey, | | 0.1 | 3,738 ± | 1.8 ± | 77,256 ± | | 13.2 ± | 24.1 ± |
| n = 3/group) | | [IV], | 314 | 0.3 | 12,459 | | 0.9 | 2.1 |
| | | 2^{nd} | | | | | | |
| | SP10477 | 0.1 | 3,945 ± | 1.6 ± | 38,458 ± | | 8.7 ± | 13.7 ± |
| | | [IV], | 871 | 0.3 | 5,369 | | 1.2 | 3 |
| | | 1^{st} | | | | | | |
| | | 0.1 | 3,619 ± | 2.2 ± | 31,567 ± | | 7.2 ± | 15.7 ± |
| | | [IV], | 832 | 0.3 | 4,097 | | 1.3 | 4 |
| | | 2^{nd} | | | | | | |
| | SP10784 | 0.3 | 13,847 ± | 1.4 ± | 308,957 ± | | 15.7 ± | 21.6 ± |
| | | [IV], | 2,552 | 0.09 | 20,245 | | 1.9 | 2 |
| | | 1^{st} | | | | | | |
| | | 0.3 | 12,052 ± | 2 ± | 221,953 ± | | 13.5 ± | 30.7 ± |
| | | [IV], | 880 | 0.4 | 39,916 | | 1.5 | 5.8 |
| | | 2^{nd} | | | | | | |
| | SP10477 | 0.3 | 11,186 ± | 1.4 ± | 107,014 ± | | 7.9 ± | 11.6 ± |
| | | [IV], | 3,042 | 0.2 | 14,755 | | 1.6 | 4 |
| | | 1^{st} | | | | | | |
| | | 0.3 | 8,702 ± | 4 ± | 78,143 ± | | 7.6 ± | 25.2 ± |
| | | [IV], | 1,397 | 0.4 | 7,867 | | 0.8 | 3 |
| | | 2^{nd} | | | | | | |
| | | Dose | | | | | | |
| | | mpk | Cmax | CL | AUC-INF | F | MRT^{a} | Vss |
| | | [ROA] | (ng/mL) | (mL/h/kg) | (h*ng/mL) | (%) | (h) | (mL/kg) |
| 21-M100- | SP10784 | 0.1 | 1,147 | 2.6 | 46,596 | 30.4 | 27 | 67.9 |
| 18597 | | [SC] | | | | | | |
| (naïve | | 0.1 | 8,532 | 0.8 | 153,886 | NA | 13.4 | 11.5 |
| rhesus | | [IV] | | | | | | |
| monkey, | | 0.3 | 3,902 | 2.8 | 164,868 | 36.2 | 32 | 139.3 |
| n = 2/group)^{a} | | [SC] | | | | | | |
| | | 0.3 | 24,537 | 1 | 456,625 | NA | 13.7 | 13.4 |
| | | [IV] | | | | | | |

^{a}Given n = 2, no standard deviation values are reported
^{b}Due to biphasic PK profiles, MRT (mean residency time) is used in place of half-life to best reflect the time drug remains in the body.

The objectives of this study had been to determine the potential toxicity, pharmacokinetics, pharmacodynamics, and tolerability of SP10784 when given by intravenous bolus injection once a week on Days one and eight to cynomolgus monkeys with a two-week observation period. Two dose levels, 0.1 mg/kg and 0.3 mg/kg were evaluated. The following parameters and endpoints were evaluated in this study: mortality, clinical observations, body weights, clinical pathology parameters (hematology and clinical chemistry), bioanalysis and toxicokinetic parameters, and immunophenotyping.

There were no definitive SP10784-related clinical signs and no SP10784-related effect on body weight over the course of this study.

SP10784-related moderate decreases in lymphocytes and basophils and minimally to mildly decreased monocytes that were marked on Days two and nine, with subsequent minimal to mild increases in lymphocytes, monocytes (at 0.3 mg/kg only) and basophils at subsequent timepoints indicated recovery following each dose. There were moderately to markedly increased eosinophils from Days nine through Day 22 (at the end of the two-week observation period.)

Dose-dependent minimal to mild decreases in RBC mass were noted on Days 2 through 8 with some recovery on Day 9 and decreased again on Days 11 through 15 and return toward baseline on Day 22. Concurrent mild increases in reticulocytes were observed, which were suggestive of increased erythropoiesis, an appropriate response to the decreases in RBC mass. While the decreases in RBC mass were considered likely SP10784-related, possibly associated with the acute phase response, blood sampling effects could not be entirely excluded in the absence of concurrent control animal data.

Platelets were minimally decreased at 0.3 mg/kg. Animals at ≥0.1 mg/kg had minimal to mild increases in platelets on Day 15. The latter were suggestive of increased thrombopoiesis associated with increased erythropoiesis at both dose levels at this time point.

Administration of SP10784 by intravenous (slow bolus) injection once a week on Days 1 and 8 was well tolerated in cynomolgus monkeys at levels of 0.1 mg/kg with no definitive adverse effects.

Table of Sequences

| SEQ ID | | |
|---|---|---|
| NO: | Description | Sequence |
| 1 | Native Human IL-2 (mature) | APTSSSTKKTQLQLEHLLLDLQMILNGIN |
| | | NYKNPKLTRMLTFKFYMPKKATELKHLQC |
| | | LEEELKPLEEVLNLAQSKNFHLRPRDLIS |
| | | NINVIVLELKGSETTFMCEYADETATIVE |
| | | FLNRWITFCQSIISTLT |
| 2 | DesA1_mature IL-2 | PTSSSTKKTQLQLEHLLLDLQMILNGINN |
| | | YKNPKLTRMLTFKFYMPKKATELKHLQCL |
| | | EEELKPLEEVLNLAQSKNFHLRPRDLISN |
| | | INVIVLELKGSETTFMCEYADETATIVEF |
| | | LNRWITFCQSIISTLT |
| 3 | Aldesleukin | PTSSSTKKTQLQLEHLLLDLQMILNGINN |
| | | YKNPKLTRMLTFKFYMPKKATELKHLQCL |
| | | EEELKPLEEVLNLAQSKNFHLRPRDLISN |
| | | INVIVLELKGSETTFMCEYADETATIVEF |
| | | LNRWITFSQSIISTLT |
| 4 | IL-2_C124S derivative 1 | MAPTSXSTKKTQLQLEHLLLDLQMILNGI |
| | X6 = nonnatural amino acid | NNXKNPXLTRMLTFKFYMPKKATELKHLQ |
| | X32 = nonnatural amino acid | CLEEELKPLEEVLNLAXSKNFHLRPRDLI |
| | X36 = nonnatural amino acid | SNINVIVLELKGSETTFMCEYADETATIV |
| | X75 = nonnatural amino acid | EFLNRWITFSQSIISTLT |
| 5 | IL-2_C124S derivative 2 | MPTSXSTKKTQLQLEHLLLDLQMILNGIN |
| | X5 = nonnatural amino acid | NXKNPXLTRMLTFKFYMPKKATELKHLQC |
| | X31 = nonnatural amino acid | LEEELKPLEEVLNLAXSKNFHLRPRDLIS |
| | X35 = nonnatural amino acid | NINVIVLELKGSETTFMCEYADETATIVE |
| | X74 = nonnatural amino acid | FLNRWITESQSIISTLT |
| 6 | IL-2_C124S derivative 3 | PTSXSTKKTQLQLEHLLLDLQMILNGINN |
| | X4 = nonnatural amino acid | XKNPXLTRMLTFKFYMPKKATELKHLQCL |
| | X30 = nonnatural amino acid | EEELKPLEEVLNLAXSKNFHLRPRDLISN |
| | X34 = nonnatural amino acid | INVIVLELKGSETTFMCEYADETATIVEF |
| | X73 = nonnatural amino acid | LNRWITFSQSIISTLT |
| 7 | IL-2_C124S derivative 4 | MAPTSXSTKKTQLQLEHLLLDLQMILNGI |
| | X6 = p-azidomethyl-L-phenylalanine | NNXKNPXLTRMLTFKFYMPKKATELKHLQ |
| | X32 = p-azidomethyl-L-phenylalanine | CLEEELKPLEEVLNLAXSKNFHLRPRDLI |
| | X36 = p-azidomethyl-L-phenylalanine | SNINVIVLELKGSETTFMCEYADETATIV |
| | X75 = p-azidomethyl-L-phenylalanine | EFLNRWITFSQSIISTLT |
| 8 | IL-2_C124S derivative 5 | MPTSXSTKKTQLQLEHLLLDLQMILNGIN |
| | X5 = p-azidomethyl-L-phenylalanine | NXKNPXLTRMLTFKFYMPKKATELKHLQC |
| | X31 = p-azidomethyl-L-phenylalanine | LEEELKPLEEVLNLAXSKNFHLRPRDLIS |
| | X35 = p-azidomethyl-L-phenylalanine | NINVIVLELKGSETTFMCEYADETATIVE |
| | X74 = p-azidomethyl-L-phenylalanine | FLNRWITFSQSIISTLT |
| 9 | IL-2_C124S derivative 6 | PTSXSTKKTQLQLEHLLLDLQMILNGINN |
| | X4 = p-azidomethyl-L-phenylalanine | XKNPXLTRMLTFKFYMPKKATELKHLQCL |
| | X30 = p-azidomethyl-L-phenylalanine | EEELKPLEEVLNLAXSKNFHLRPRDLISN |
| | X34 = p-azidomethyl-L-phenylalanine | INVIVLELKGSETTFMCEYADETATIVEF |
| | X73 = p-azidomethyl-L-phenylalanine | LNRWITFSQSIISTLT |
| 10 | IL-2_C124S derivative 7 | MAPTSXSTKKTQLQLEHLLLDLQMILNGI |
| | X6 = p-azidomethyl-L-phenylalanine | NNXKNPXLTRMLTFKFYMPKKATELKHLQ |
| | conjugated to nonpeptide, water-soluble | CLEEELKPLEEVLNLAXSKNFHLRPRDLI |
| | polymer via a releasable linker | SNINVIVLELKGSETTFMCEYADETATIV |
| | X32 = p-azidomethyl-L-phenylalanine | EFLNRWITFSQSIISTLT |
| | conjugated to nonpeptide, water-soluble | |
| | polymer via a releasable linker | |
| | X36 = p-azidomethyl-L-phenylalanine | |
| | conjugated to nonpeptide, water-soluble | |
| | polymer via a releasable linker | |
| | X75 = p-azidomethyl-L-phenylalanine | |
| | conjugated to nonpeptide, water-soluble | |
| | polymer via a releasable linker | |
| 11 | IL-2_C124S derivative 8 | MPTSXSTKKTQLQLEHLLLDLQMILNGIN |
| | X5 = p-azidomethyl-L-phenylalanine | NXKNPXLTRMLTFKFYMPKKATELKHLQC |
| | conjugated to nonpeptide, water-soluble | LEEELKPLEEVLNLAXSKNFHLRPRDLIS |
| | polymer via a releasable linker | NINVIVLELKGSETTFMCEYADETATIVE |
| | X31 = p-azidomethyl-L-phenylalanine | FLNRWITFSQSIISTLT |
| | conjugated to nonpeptide, water-soluble | |
| | polymer via a releasable linker | |
| | X35 = p-azidomethyl-L-phenylalanine | |
| | conjugated to nonpeptide, water-soluble | |
| | polymer via a releasable linker | |
| | X74 = p-azidomethyl-L-phenylalanine | |
| | conjugated to nonpeptide, water-soluble | |
| | polymer via a releasable linker | |
| 12 | IL-2_C124S derivative 9 | PTSXSTKKTQLQLEHLLLDLQMILNGINN |
| | X4 = p-azidomethyl-L-phenylalanine | XKNPXLTRMLTFKFYMPKKATELKHLQCL |
| | conjugated to nonpeptide, water-soluble | EEELKPLEEVLNLAXSKNFHLRPRDLISN |
| | polymer via a releasable linker | INVIVLELKGSETTFMCEYADETATIVEF |
| | X30 = p-azidomethyl-L-phenylalanine | LNRWITFSQSIISTLT |
| | conjugated to nonpeptide, water-soluble | |
| | polymer via a releasable linker | |
| | X34 = p-azidomethyl-L-phenylalanine | |
| | conjugated to nonpeptide, water-soluble | |
| | polymer via a releasable linker | |
| | X74 = p-azidomethyl-L-phenylalanine | |
| | conjugated to nonpeptide, water-soluble | |
| | polymer via a releasable linker | |
| 13 | IL-2_C124S derivative 10 | MAPTSXSTKKTQLQLEHLLLDLQMILNGI |
| | X6 = p-azidomethyl-L-phenylalanine | NNXKNPXLTRMLTFKFYMPKKATELKHLQ |
| | conjugated to PEG via a releasable linker | CLEEELKPLEEVLNLAXSKNFHLRPRDLI |
| | X32 = p-azidomethyl-L-phenylalanine | SNINVIVLELKGSETTFMCEYADETATIV |
| | conjugated to PEG via a releasable linker | EFLNRWITFSQSIISTLT |
| | X36 = p-azidomethyl-L-phenylalanine | |
| | conjugated to PEG via a releasable linker | |
| | X75 = p-azidomethyl-L-phenylalanine | |
| | conjugated to PEG via a releasable linker | |
| 14 | IL-2_C124S derivative 11 | MPTSXSTKKTQLQLEHLLLDLQMILNGIN |
| | X5 = p-azidomethyl-L-phenylalanine | NXKNPXLTRMLTFKFYMPKKATELKHLQC |
| | conjugated to PEG via a releasable linker | LEEELKPLEEVLNLAXSKNFHLRPRDLIS |
| | X31 = p-azidomethyl-L-phenylalanine | NINVIVLELKGSETTFMCEYADETATIVE |
| | conjugated to PEG via a releasable linker | FLNRWITFSQSIISTLT |
| | X35 = p-azidomethyl-L-phenylalanine | |
| | conjugated to PEG via a releasable linker | |
| | X74 = p-azidomethyl-L-phenylalanine | |
| | conjugated to PEG via a releasable linker | |
| 15 | IL-2_C124S derivative 12 | PTSXSTKKTQLQLEHLLLDLQMILNGINN |
| | X4 = p-azidomethyl-L-phenylalanine | XKNPXLTRMLTFKFYMPKKATELKHLQCL |
| | conjugated to PEG via a releasable linker | EEELKPLEEVLNLAXSKNFHLRPRDLISN |
| | X30 = p-azidomethyl-L-phenylalanine | INVIVLELKGSETTFMCEYADETATIVEF |
| | conjugated to PEG via a releasable linker | LNRWITFSQSIISTLT |
| | X34 = p-azidomethyl-L-phenylalanine | |
| | conjugated to PEG via a releasable linker | |
| | X73 = p-azidomethyl-L-phenylalanine | |
| | conjugated to PEG via a releasable linker | |
| 16 | IL-2_C124S derivative 13 | MAPTSXSTKKTQLQLEHLLLDLQMILNGI |
| | X6 = p-azidomethyl-L-phenylalanine | NNXKNPXLTRMLTFKFYMPKKATELKHLQ |
| | conjugated to SC579 | CLEEELKPLEEVLNLAXSKNFHLRPRDLI |
| | X32 = p-azidomethyl-L-phenylalanine | SNINVIVLELKGSETTFMCEYADETATIV |
| | conjugated to SC579 | EFLNRWITFSQSIISTLT |
| | X36 = p-azidomethyl-L-phenylalanine | |
| | conjugated to SC579 | |
| | X75 = p-azidomethyl-L-phenylalanine | |
| | conjugated to SC579 | |
| 17 | IL-2_C124S derivative 14 (SP10784) | MPTSXSTKKTQLQLEHLLLDLQMILNGIN |
| | X5 = p-azidomethyl-L-phenylalanine | NXKNPXLTRMLTFKFYMPKKATELKHLQC |
| | conjugated to SC579 | LEEELKPLEEVLNLAXSKNFHLRPRDLIS |
| | X31 = p-azidomethyl-L-phenylalanine | NINVIVLELKGSETTFMCEYADETATIVE |
| | conjugated to SC579 | FLNRWITFSQSIISTLT |
| | X35 = p-azidomethyl-L-phenylalanine | |
| | conjugated to SC579 | |
| | X74 = p-azidomethyl-L-phenylalanine | |
| | conjugated to SC579 | |
| 18 | IL-2_C124S derivative 15 | PTSXSTKKTQLQLEHLLLDLQMILNGINN |
| | X4 = p-azidomethyl-L-phenylalanine | XKNPXLTRMLTFKFYMPKKATELKHLQCL |
| | conjugated to SC579 | EEELKPLEEVLNLAXSKNFHLRPRDLISN |
| | X30 = p-azidomethyl-L-phenylalanine | INVIVLELKGSETTFMCEYADETATIVEF |
| | conjugated to SC579 | LNRWITFSQSIISTLT |
| | X34 = p-azidomethyl-L-phenylalanine | |
| | conjugated to SC579 | |
| | X73 = p-azidomethyl-L-phenylalanine | |
| | conjugated to SC579 | |
| 19 | Aldesleukin-6His | MPTSSSTKKTQLQLEHLLLDLQMILNGIN |
| | | NYKNPKLTRMLTFKFYMPKKATELKHLQC |
| | | LEEELKPLEEVLNLAQSKNFHLRPRDLIS |
| | | NINVIVLELKGSETTFMCEYADETATIVE |
| | | FLNRWITESQSIISTLTGGSHHHHHH |
| 20 | Aldesleukin-6His S4TAG p-azidomethyl-L- | MPTSXSTKKTQLQLEHLLLDLQMILNGIN |
| | phenylalanine (SP9954) | NYKNPKLTRMLTFKFYMPKKATELKHLQC |
| | X5 is pAMF | LEEELKPLEEVLNLAQSKNFHLRPRDLIS |
| | | NINVIVLELKGSETTFMCEYADETATIVE |
| | | FLNRWITFSQSIISTLTGGSHHHHHH |
| 21 | Aldesleukin-6His Y30TAG p-azidomethyl- | MPTSSSTKKTQLQLEHLLLDLQMILNGIN |
| | L-phenylalanine (SP9957) | NXKNPKLTRMLTFKFYMPKKATELKHLQC |
| | X31 is pAMF | LEEELKPLEEVLNLAQSKNFHLRPRDLIS |
| | | NINVIVLELKGSETTFMCEYADETATIVE |
| | | FLNRWITFSQSIISTLTGGSHHHHHH |
| 22 | Aldesleukin-6His K34TAG p-azidomethyl- | MPTSSSTKKTQLQLEHLLLDLQMILNGIN |
| | L-phenylalanine (SP9833) | NYKNPXLTRMLTFKFYMPKKATELKHLQC |
| | X35 is pAMF | LEEELKPLEEVLNLAQSKNFHLRPRDLIS |
| | | NINVIVLELKGSETTFMCEYADETATIVE |
| | | FLNRWITFSQSIISTLTGGSHHHHHH |
| 23 | Aldesleukin-6His F41TAG p-azidomethyl- | MPTSSSTKKTQLQLEHLLLDLQMILNGIN |
| | L-phenylalanine (SP9960) | NYKNPKLTRMLTXKFYMPKKATELKHLQC |
| | X42 is pAMF | LEEELKPLEEVLNLAQSKNFHLRPRDLIS |
| | | NINVIVLELKGSETTFMCEYADETATIVE |
| | | FLNRWITFSQSIISTLTGGSHHHHHH |
| 24 | Aldesleukin-6His Q73TAG p-azidomethyl- | MPTSSSTKKTQLQLEHLLLDLQMILNGIN |
| | L-phenylalanine (SP9965) | NYKNPKLTRMLTFKFYMPKKATELKHLQC |
| | X74 is pAMF | LEEELKPLEEVLNLAXSKNFHLRPRDLIS |
| | | NINVIVLELKGSETTFMCEYADETATIVE |
| | | FLNRWITFSQSIISTLTGGSHHHHHH |
| 25 | Aldesleukin-6His F77TAG p-azidomethyl- | MPTSSSTKKTQLQLEHLLLDLQMILNGIN |
| | L-phenylalanine (SP9942) | NYKNPKLTRMLTFKFYMPKKATELKHLQC |
| | X78 is pAMF | LEEELKPLEEVLNLAQSKNXHLRPRDLIS |
| | | NINVIVLELKGSETTFMCEYADETATIVE |
| | | FLNRWITESQSIISTLTGGSHHHHHH |
| 26 | Aldesleukin-6His R80TAG p-azidomethyl- | MPTSSSTKKTQLQLEHLLLDLQMILNGIN |
| | L-phenylalanine (SP9968) | NYKNPKLTRMLTFKFYMPKKATELKHLQC |
| | X81 is pAMF | LEEELKPLEEVLNLAQSKNFHLXPRDLIS |
| | | NINVIVLELKGSETTFMCEYADETATIVE |
| | | FLNRWITFSQSIISTLTGGSHHHHHH |
| 27 | Aldesleukin-6His V114TAG | MPTSSSTKKTQLQLEHLLLDLQMILNGIN |
| | p-azidomethyl-L-phenylalanine (SP9970) | NYKNPKLTRMLTFKFYMPKKATELKHLQC |
| | X115 is pAMF | LEEELKPLEEVLNLAQSKNFHLRPRDLIS |
| | | NINVIVLELKGSETTFMCEYADETATIXE |
| | | FLNRWITESQSIISTLTGGSHHHHHH |
| 28 | Aldesleukin F41TAG p-azidomethyl-L- | MPTSSSTKKTQLQLEHLLLDLQMILNGINNY |
| | phenylalanine (SP10098) | KNPKLTRMLTXKFYMPKKATELKHLQCLEEE |
| | X42 is pAMF | LKPLEEVLNLAQSKNFHLRPRDLISNINVIV |
| | | LELKGSETTFMCEYADETATIVEFLNRWITF |
| | | SQSIISTLT |
| 29 | Aldesleukin K34/F41TAG p-azidomethyl- | MPTSSSTKKTQLQLEHLLLDLQMILNGINNY |
| | L-phenylalanine (SP10286) | KNPXLTRMLTXKFYMPKKATELKHLQCLEEE |
| | X35 | LKPLEEVLNLAQSKNFHLRPRDLISNINVIV |
| | | LELKGSETTFMCEYADETATIVEFLNRWITF |
| | | SQSIISTLT |
| 30 | Aldesleukin F41/K34/Y30TAG | MPTSSSTKKTQLQLEHLLLDLQMILNGINNX |
| | p-azidomethyl-L-phenylalanine (SP10280) | KNPXLTRMLTXKFYMPKKATELKHLQCLEEE |
| | X31 is pAMF | LKPLEEVLNLAQSKNFHLRPRDLISNINVIV |
| | X35 is pAMF | LELKGSETTFMCEYADETATIVEFLNRWITF |
| | X42 is pAMF | SQSIISTLT |
| 31 | Aldesleukin_S4/Y30/K34TAG | MPTSXSTKKTQLQLEHLLLDLQMILNGINNX |
| | p-azidomethyl-L-phenylalanine (SP10480) | KNPXLTRMLTFKFYMPKKATELKHLQCLEEE |
| | X5 is pAMF | LKPLEEVLNLAQSKNFHLRPRDLISNINVIV |
| | X31 is pAMF | LELKGSETTFMCEYADETATIVEFLNRWITF |
| | X35 is pAMF | SQSIISTLT |
| 32 | Aldesleukin_R37A/F41K_S4/Y30/K34TAG | MPTSXSTKKTQLQLEHLLLDLQMILNGINNX |
| | p-azidomethyl-L-phenylalanine (SP10481) | KNPXLTAMLTKKFYMPKKATELKHLQCLEEE |
| | X5 is pAMF | LKPLEEVLNLAQSKNFHLRPRDLISNINVIV |
| | X31 is pAMF | LELKGSETTFMCEYADETATIVEFLNRWITF |
| | X35 is pAMF | SQSIISTLT |
| 33 | Aldesleukin-6His_S4/K34/Q73TAG | MPTSXSTKKTQLQLEHLLLDLQMILNGINNY |
| | p-azidomethyl-L-phenylalanine (SP10681) | KNPXLTRMLTFKFYMPKKATELKHLQCLEEE |
| | X5 is pAMF | LKPLEEVLNLAQSKNXHLRPRDLISNINVIV |
| | X35 is pAMF | LELKGSETTFMCEYADETATIVEFLNRWITF |
| | X74 is pAMF | SQSIISTLTGGSHHHHHH |
| 34 | Aldesleukin-6His_S4/K34/F77TAG | MPTSXSTKKTQLQLEHLLLDLQMILNGINNY |
| | p-azidomethyl-L-phenylalanine (SP10682) | KNPXLTRMLTFKFYMPKKATELKHLQCLEEE |
| | X5 is pAMF | LKPLEEVLNLAQSKNXHLRPRDLISNINVIV |
| | X35 is pAMF | LELKGSETTFMCEYADETATIVEFLNRWITF |
| | X78 is pAMF | SQSIISTLTGGSHHHHHH |
| 35 | Aldesleukin-6His_S4/K34/V114TAG | MPTSXSTKKTQLQLEHLLLDLQMILNGINNY |
| | p-azidomethyl-L-phenylalanine (SP10683) | KNPXLTRMLTFKFYMPKKATELKHLQCLEEE |
| | X5 is pAMF | LKPLEEVLNLAQSKNFHLRPRDLISNINVIV |
| | X35 is pAMF | LELKGSETTFMCEYADETATIXEFLNRWITF |
| | X115 is pAMF | SQSIISTLTGGSHHHHHH |
| 36 | Aldesleukin-6His_S4/Y30/K34TAG F41K | MPTSXSTKKTQLQLEHLLLDLQMILNGINNX |
| | p-azidomethyl-L-phenylalanine (SP10684) | KNPXLTRMLTKKFYMPKKATELKHLQCLEEE |
| | X5 is pAMF | LKPLEEVLNLAQSKNFHLRPRDLISNINVIV |
| | X31 is pAMF | LELKGSETTFMCEYADETATIVEFLNRWITF |
| | X35 is pAMF | SQSIISTLTGGSHHHHHH |
| 37 | Aldesleukin-6His_S4/Y30/K34/R80TAG | MPTSXSTKKTQLQLEHLLLDLQMILNGINNX |
| | p-azidomethyl-L-phenylalanine (SP10685) | KNPXLTRMLTFKFYMPKKATELKHLQCLEEE |
| | X5 is pAMF | LKPLEEVLNLAQSKNFHLXPRDLISNINVIV |
| | X31 is pAMF | LELKGSETTFMCEYADETATIVEFLNRWITF |
| | X35 is pAMF | SQSIISTLTGGSHHHHHH |
| | X81 is pAMF | |
| 38 | Aldesleukin-6His_S4/Y30/K34/Q73TAG | MPTSXSTKKTQLQLEHLLLDLQMILNGINNX |
| | p-azidomethyl-L-phenylalanine (SP10686) | KNPXLTRMLTFKFYMPKKATELKHLQCLEEE |
| | X5 is pAMF | LKPLEEVLNLAXSKNFHLRPRDLISNINVIV |
| | X31 is pAMF | LELKGSETTFMCEYADETATIVEFLNRWITF |
| | X35 is pAMF | SQSIISTLTGGSHHHHHH |
| | X74 is pAMF | |
| 39 | Aldesleukin-6His_S4/Y30/K34/V114TAG | MPTSXSTKKTQLQLEHLLLDLQMILNGINNX |
| | p-azidomethyl-L-phenylalanine (SP10687) | KNPXLTRMLTFKFYMPKKATELKHLQCLEEE |
| | X5 is pAMF | LKPLEEVLNLAQSKNFHLRPRDLISNINVIV |
| | X31 is pAMF | LELKGSETTFMCEYADETATIXEFLNRWITF |
| | X35 is pAMF | SQSIISTLTGGSHHHHHH |
| | X115 is pAMF | |
| 40 | Aldesleukin-6His_S4/Y30/K34/R80TAG | MPTSXSTKKTQLQLEHLLLDLQMILNGINNX |
| | F41K p-azidomethyl-L-phenylalanine | KNPXLTRMLTKKFYMPKKATELKHLQCLEEE |
| | (SP10688) | LKPLEEVLNLAQSKNFHLXPRDLISNINVIV |
| | X5 is pAMF | LELKGSETTFMCEYADETATIVEFLNRWITF |
| | X31 is pAMF | SQSIISTLTGGSHHHHHH |
| | X35 is pAMF | |
| | X81 is pAMF | |
| 41 | Aldesleukin_S4/K34/V114TAG | MPTSXSTKKTQLQLEHLLLDLQMILNGINNY |
| | p-azidomethyl-L-phenylalanine (SP10774) | KNPXLTRMLTFKFYMPKKATELKHLQCLEEE |
| | X5 is pAMF | LKPLEEVLNLAQSKNFHLRPRDLISNINVIV |
| | X35 is pAMF | LELKGSETTFMCEYADETATIXEFLNRWITF |
| | X115is pAMF | SQSIISTLT |
| 42 | Aldesleukin_S4/Y30/K34/Q73TAG | MPTSXSTKKTQLQLEHLLLDLQMILNGINNX |
| | p-azidomethyl-L-phenylalanine (SP10776) | KNPXLTRMLTFKFYMPKKATELKHLQCLEEE |
| | X5 is pAMF | LKPLEEVLNLAXSKNFHLRPRDLISNINVIV |
| | X31 is pAMF | LELKGSETTFMCEYADETATIVEFLNRWITF |
| | X35 is pAMF | SQSIISTLT |
| | X74 is pAMF | |
| 43 | Aldesleukin_S4/Y30/K34/V114TAG | MPTSXSTKKTQLQLEHLLLDLQMILNGINNX |
| | p-azidomethyl-L-phenylalanine | KNPXLTRMLTFKFYMPKKATELKHLQCLEEE |
| | (SP10777) | LKPLEEVLNLAQSKNFHLRPRDLISNINVIV |
| | X5 is pAMF | LELKGSETTFMCEYADETATIXEFLNRWITF |
| | X31 is pAMF | SQSIISTLT |
| | X35 is pAMF | |
| | X115 is pAMF | |
| 44 | HIS6 Tag | HHHHHH |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. (canceled)

2. An interleukin 2 (IL-2) conjugate comprising:
an IL-2 polypeptide comprising an amino acid sequence with at least 80% identity to the amino acid sequence set forth in SEQ ID NO: 2 or SEQ ID NO: 3,
wherein the amino acids in at least three of positions S4, Y30, K34, and Q73 of the IL-2 polypeptide in reference to the amino acid positions within SEQ ID NO: 2 are each substituted with a nonnatural amino acid (NNAA) conjugated to a nonpeptidic, water-soluble polymer by a releasable linker comprising a degradable linkage.

3-5. (canceled)

6. The IL-2 conjugate of claim 2, wherein the NNAA is selected from the group consisting of p-azidomethyl-L-phenylalanine, p-azido-L-phenylalanine, p-acetyl-L-phenylalanine, N6-azidoethoxy-L-lysine, N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc-serine, L-Dopa, fluorinated phenylalanine, isopropyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, p-propargyloxy-phenylalanine, 2-amino-3-((2-((3-(benzyloxy)-3-oxopropyl)amino)ethyl)selanyl)propanoic acid, 2-amino-3-(phenylselanyl)propanoic, selenocysteine, m-acetylphenylalanine, 2-amino-8-oxononanoic acid, and p-propargyloxyphenylalanine.

7-8. (canceled)

9. The IL-2 conjugate of claim 2, wherein the NNAA is p-azidomethyl-L-phenylalanine.

10-11. (canceled)

12. The IL-2 conjugate of claim 2, wherein the nonpeptidic, water-soluble polymer is polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof.

13. (canceled)

14. The IL-2 conjugate of claim 2, wherein the releasable linker comprises a fluorenylmethyloxycarbonyl (Fmoc) group covalently linked to the nonpeptidic, water-soluble polymer and to the reactive group to provide a releasable linker comprising a degradable linkage.

15. The IL-2 conjugate of claim 2, wherein releasable linker is selected from a compound of Formula (XXXII): 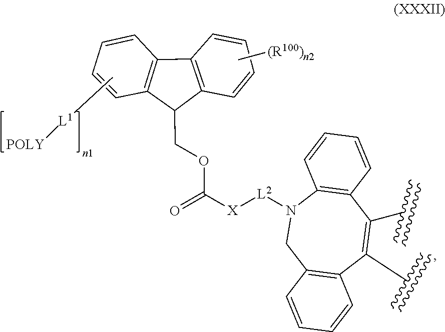
wherein the double wavy lines indicate attachment to a moiety of the non-natural amino acid residue;
POLY is a nonpeptidic, water-soluble polymer;
L¹ and L² are independently selected from the group consisting of —O—C₁-C₆alkylene-NH—C(O)—C₁-C₆alkylene-C(O)—NH—, —O—C₁-C₆alkylene-NH—C(O)—C₁-C₆alkylene-, —O—C₁-C₆alkylene-NH—C(O)—C₁-C₆alkylene-C(O)—NH—C₁-C₆alkylene-, —O—C₁-C₆alkylene-NH—C(O)—, and —C₁-C₆alkylene-C(O)—, wherein each —C₁-C₆alkylene- is independently optionally substituted with one or more substituents independently selected from halogen, alkyl, haloalkyl, hydroxyl, amino, alkylamino, and alkoxy;
X is a bond, —O—, or —N(R²)—;
each R¹⁰⁰ is hydrogen or lower alkyl;
n1 is an integer selected from one to two; and
n2 is an integer selected from one to four.

16. The IL-2 conjugate of claim 15, wherein L¹ is —O—C₁-C₆alkylene-NH—C(O)—.

17. The IL-2 conjugate of claim 15, wherein L² is —C₁-C₆alkylene-C(O)—.

18. The IL-2 conjugate of claim 15, wherein n1 is 1.

19. The IL-2 conjugate of claim 15, wherein R¹⁰⁰ is hydrogen.

20. The IL-2 conjugate of claim 15, wherein n2 is 1.

21-26. (canceled)

27. The IL-2 conjugate of claim 2, wherein the amino acid set forth in SEQ ID NO: 2 further comprises at the N-terminus a methionine residue, an alanine residue, or a methionine alanine dipeptide.

28. The IL-2 conjugate of claim 2, wherein the IL-2 conjugate comprises the amino acid sequence set forth in SEQ ID NO: 10, 11, or 12.

29. The IL-2 conjugate of claim 2, wherein the IL-2 conjugate comprises the amino acid sequence set forth in SEQ ID NO: 13, 14, or 15.

30. The IL-2 conjugate of claim 2, wherein the IL-2 conjugate comprises the amino acid sequence set forth in SEQ ID NO: 16, 17, or 18.

31. The IL-2 conjugate of claim 2, wherein the IL-2 conjugate has undetectable or significantly attenuated binding to the IL-2α and IL-2 β receptors when each NNAA is covalently linked to the nonpeptidic, water-soluble polymer by the releasable linker as determined by surface plasmon resonance when compared to binding of an IL-2 polypeptide not covalently linked to the nonpeptidic, water-soluble polymer.

32-40. (canceled)

41. A composition comprising:
the IL-2 conjugate of claim 2 and a pharmaceutically acceptable carrier or excipient.

42. (canceled)

43. A method for treating a proliferative disease or cancer in an individual, comprising:
administering a therapeutically effective amount of the IL-2 conjugate of claim 2 or composition thereof comprising a pharmaceutically acceptable carrier or excipient to an individual in need thereof to treat the proliferative disease or cancer in the individual.

44. A combination therapy for treating a proliferative disease or cancer in an individual, comprising:
administering a therapeutically effective amount of the IL-2 conjugate claim 2 or composition thereof comprising a pharmaceutically acceptable carrier or excipient to an individual in need thereof, and
administering a therapeutically effective amount of a therapeutic agent to the individual, to treat the proliferative disease or cancer in the individual.

45. The combination therapy of claim 44, wherein the therapeutic agent is an anti-PD1 antibody or anti-PDL1 antibody.

46-70. (canceled)
